SEQUENCE LISTING (25 sequences)

SEQ ID NO: 1 - DNA, 20 bp, Artificial Sequence
  Description of Artificial Sequence Synthetic oligonucleotide
gacggctgaggagcggaaga

SEQ ID NO: 2 - DNA, 20 bp, Artificial Sequence
  Description of Artificial Sequence Synthetic oligonucleotide
tgtggaggtgagcaatcccc

SEQ ID NO: 3 - DNA, 20 bp, Artificial Sequence
  Description of Artificial Sequence Synthetic oligonucleotide
tcttccgctcctcagccgtc

SEQ ID NO: 4 - DNA, 20 bp, Artificial Sequence
  Description of Artificial Sequence Synthetic oligonucleotide
cggcaagacgcggaagctca

SEQ ID NO: 5 - DNA, 20 bp, Artificial Sequence
  Description of Artificial Sequence Synthetic oligonucleotide
acagatatggcaactcccag

SEQ ID NO: 6 - DNA, 20 bp, Artificial Sequence
  Description of Artificial Sequence Synthetic oligonucleotide
tatcatgtcgttattaactg

SEQ ID NO: 7 - DNA, 20 bp, Artificial Sequence
  Description of Artificial Sequence Synthetic oligonucleotide
tcacaaaatttacacagttg

SEQ ID NO: 8 - DNA, 20 bp, Artificial Sequence
  Description of Artificial Sequence Synthetic oligonucleotide
gcaggatttctggttgtcac

SEQ ID NO: 9 - DNA, 20 bp, Artificial Sequence
  Description of Artificial Sequence Synthetic oligonucleotide
cccaccgcacgttcagaagt

SEQ ID NO: 10 - DNA, 20 bp, Artificial Sequence
  Description of Artificial Sequence Synthetic primer
aacccacaacgaaatctatg

SEQ ID NO: 11 - DNA, 20 bp, Artificial Sequence
  Description of Artificial Sequence Synthetic primer
cttttaacttgagcctcagc

SEQ ID NO: 12 - DNA, 18 bp, Artificial Sequence
  Description of Artificial Sequence Synthetic primer
aacccgttgaaccccatt

SEQ ID NO: 13 - DNA, 20 bp, Artificial Sequence
  Description of Artificial Sequence Synthetic primer
ccatccaatcggtagtagcg

SEQ ID NO: 14 - DNA, 20 bp, Artificial Sequence
  Description of Artificial Sequence Synthetic oligonucleotide
gaatcggagccatagtccaa

SEQ ID NO: 15 - DNA, 20 bp, Artificial Sequence
  Description of Artificial Sequence Synthetic oligonucleotide
gcattcaagtgctggtccgg

SEQ ID NO: 16 - DNA, 20 bp, Artificial Sequence
  Description of Artificial Sequence Synthetic oligonucleotide
ccacgtctaggttgaaggcg

SEQ ID NO: 17 - DNA, 20 bp, Artificial Sequence
  Description of Artificial Sequence Synthetic oligonucleotide
gacggctgaggagcggaaga

SEQ ID NO: 18 - DNA, 20 bp, Artificial Sequence
  Description of Artificial Sequence Synthetic oligonucleotide
tgtggaggtgagcaatcccc

SEQ ID NO: 19 - DNA, 23 bp, Artificial Sequence
  Description of Artificial Sequence Synthetic oligonucleotide
ccttgagaagcgacagccagtga

SEQ ID NO: 20 - DNA, 24 bp, Artificial Sequence
  Description of Artificial Sequence Synthetic oligonucleotide
tcactggctgtcggcttctcaagg

SEQ ID NO: 21 - DNA, 23 bp, Artificial Sequence
  Description of Artificial Sequence Synthetic oligonucleotide
cctggccagactggcaccaacgg

SEQ ID NO: 22 - DNA, 23 bp, Artificial Sequence
  Description of Artificial Sequence Synthetic oligonucleotide
ccgttggtgccagtctggccagg

SEQ ID NO: 23 - DNA, 20 bp, Artificial Sequence
  Description of Artificial Sequence Synthetic oligonucleotide
acggccacgcagacttcatg

SEQ ID NO: 24 - DNA, 20 bp, Artificial Sequence
  Description of Artificial Sequence Synthetic oligonucleotide
ggacttctggttgtcgcaag

SEQ ID NO: 25 - protein, 4 aa, Artificial Sequence
  Description of Artificial Sequence Synthetic peptide
RGDS

(19) United States
(12) Patent Application Publication
(10) Pub. No.: US 2022/0378739 A1
(43) Pub. Date: Dec. 1, 2022

(54) NATURAL KILLER CELL IMMUNOTHERAPY FOR THE TREATMENT OF GLIOBLASTOMA AND OTHER CANCERS

(71) Applicant: BOARD OF REGENTS, THE UNIVERSITY OF TEXAS SYSTEM, Austin, TX (US)

(72) Inventors: Katy REZVANI, Houston, TX (US); Mayra SHANLEY, Houston, TX (US); Elizabeth SHPALL, Houston, TX (US); David MARIN COSTA, Houston, TX (US); Rafet BASAR, Houston, TX (US); Hila SHAIM, Houston, TX (US)

(21) Appl. No.: 17/755,881

(22) PCT Filed: Nov. 25, 2020
(86) PCT No.: PCT/US2020/062381

§ 371 Date: May 11, 2022

Related U.S. Application Data
(60) Provisional application No. 63/022,936, filed on May 11, 2020.
(60) Provisional application No. 62/941,050, filed on Nov. 27, 2019.

(51) Int. Cl. A61K 31/395; A61K 35/17; C12N 5/0783

(52) U.S. Cl. CPC A61K 31/395; A61K 35/17; C12N 5/0646; C12N 2501/15; C12N 2502/1164

(57) ABSTRACT

Embodiments of the disclosure provide methods and compositions that facilitate cancer treatment including at least because they concern therapies that circumvent the tumor microenvironment. In specific embodiments, compositions are utilized for therapy that utilize NK cells that are protected from the direct inhibition of their activity (using TGF-beta inhibitors) and/or that are indirectly protected from TGF-beta (using integrin inhibitors). In specific embodiments, the NK cells have deficient expression and/or activity for TGF-beta Receptor 2 and/or glucocorticoid receptor.

This application claims priority to U.S. Provisional Patent Application Ser. No. 62/941,050, filed Nov. 27, 2019, and also claims priority to U.S. Provisional Patent Application Ser. No. 63/022,936, filed May 11, 2020, both of which applications are incorporated by reference herein in its entirety.

### SEQUENCE LISTING

The instant application contains a Sequence Listing which has been submitted electronically in ASCII format and is hereby incorporated by reference in its entirety. Said ASCII copy, created on Nov. 12, 2020, is named UTSC_P1189WO_SL.txt and is 6,175 bytes in size.

### TECHNICAL FIELD

Embodiments of the disclosure concern at least the fields of cell biology, molecular biology, immunology, and medicine.

### BACKGROUND

Many malignancies either lack antigenic targets or have heterogeneous peptide expression that results in relapse secondary to antigen escape variants. These malignancies are also refractory to immune checkpoint inhibitors and lack expression of the major histocompatibility complex (MHC). Natural killer (NK) cells may be more suitable as therapeutic effectors against highly heterogeneous solid tumors such as glioblastoma (GBM), because unlike T and B lymphocytes, they do not possess rearranged V(D)J receptors and are not restricted by MHC-bound antigen presentation, which is downregulated in many solid tumors. Instead, their effector function is dictated by the integration of signals received through germline-encoded receptors that can recognize multiple ligands on cancer targets without particular antigen specificity or requirement for co-stimulation. However, NK cells become irreversibly, immunologically unresponsive owing to tumor elaborated TGF-beta as well as other immunosuppressive molecules. The present disclosure provides solutions for addressing the inhibitor effects on NK cells by TGF-beta and associated molecules.

### BRIEF SUMMARY

Embodiments of the disclosure encompass methods and compositions for immunotherapy cancer treatment and, in certain cases, prevention. The disclosure in particular provides methods and compositions to allow NK cells to be more effective for cancer treatment than in the absence of the disclosed methods and compositions. In specific embodiments, the disclosure provides methods and compositions to allow NK cells to be more effective in tumor microenvironments compared to use of NK cells in the absence of the disclosed methods and compositions.

The disclosure provides multiple approaches to improvements on cancer immunotherapy, particularly with respect to use of NK cells of any kind, and the separate approaches may or may not be used in conjunction with one another. In one aspect of the disclosure, NK cell immunotherapy is used either alone or in conjunction with one or more integrin inhibitors. In another aspect of the disclosure, NK cell immunotherapy is used in conjunction with one or more TGF-beta inhibitors. In an additional aspect of the disclosure NK cells are used for immunotherapy that are gene-edited for the TGF-beta R2 gene, such as disruption of expression and/or activity. In specific embodiments, the immunotherapy comprises a mixture of NK cells wherein in the mixture of NK cells, a plurality comprises downregulation or knockout of TGF-beta R2 gene and/or glucocorticoid receptor (the NR3C1 gene) and the plurality also comprises NK cells that may be non-transduced or that may be engineered for a different purpose. In such cases, the NK cells in the mixture having downregulation or knockout of TGF-beta R2 gene (with or without the NR3C1 gene) are effective enough at the tumor microenvironment and/or at TGF-beta inhibition of NK cells to allow anti-cancer efficacy of other types of NK cells, whether or not they are also engineered. The NK cells may be modified in one, two, or more ways of any of the aforementioned modifications or any encompassed herein.

In specific embodiments, the NK cells are gene-edited with respect to the TGF-beta R2 gene. The TGF-beta R2 gene may be edited by CRISPR/Cas gene editing technology, as only one example. Examples of sequences used for guide RNAs may comprise one or more of SEQ ID NOs: 1-9 and 23-24:

SEQ ID NO: 1:
GACGGCTGAGGAGCGGAAGA
SEQ ID NO: 2:
TGTGGAGGTGAGCAATCCCC
SEQ ID NO: 3
TCTTCCGCTCCTCAGCCGTC
SEQ ID NO: 4
CGGCAAGACGCGGAAGCTCA
SEQ ID NO: 5
ACAGATATGGCAACTCCCAG
SEQ ID NO: 6
TATCATGTCGTTATTAACTG
SEQ ID NO: 7
TCACAAAATTTACACAGTTG
SEQ ID NO: 8
GCAGGATTTCTGGTTGTCAC
SEQ ID NO: 9
CCCACCGCACGTTCAGAAGT
Mouse sequences:
Mm.Cas9.TGFBR2.1.AA:
(SEQ ID NO: 23)
ACGGCCACGCAGACTTCATG
Mm.Cas9.TGFBR2.1.AB:
(SEQ ID NO: 24)
GGACTTCTGGTTGTCGCAAG

Particular embodiments encompass immunotherapy with ex vivo-expanded and activated NK cells in combination with one or more TGF-beta inhibitors and/or one or more integrin inhibitors and/or genetically engineered NK cells with TGF-beta receptor 2 knockout (KO) and/or GR KO (with or without expression of one or more engineered receptors (such as chimeric antigen receptors (CAR) and/or synthetic T cell receptors) and/or one or more cytokine gene(s)). Such immunotherapy may be utilized in individuals with any type of solid tumor or hematologic malignancy. In specific cases, such immunotherapy is for an individual with glioblastoma. In particular embodiments, the NK cells are allogeneic NK cells with respect to an individual. In addition, the use of allogeneic NK cells in combination with one or more TGF-beta inhibitors and/or one or more integrin inhibitors and/or NK cells genetically with TGF-beta receptor 2 KO and/or GR KO (with or without expression of one or more engineered receptor(s) and/or one or more cytokine gene(s)) provides an off-the-shelf therapy that extends therapy to multiple individuals.

Embodiments include compositions comprising two or more of (a), (b), (c), and (d) as follows: (a) one or both of (1) and (2): (1) one or more compounds that disrupt expression or activity of transforming growth factor (TGF)-beta receptor 2 (TGFBR2); (2) natural killer (NK) cells comprising a disruption of expression or activity of TGFBR2 endogenous to the immune cells; (b) one or both of (1) and (2): (1) one or more compounds that disrupt expression or activity of glucocorticoid receptor (GR); (2) natural killer (NK) cells comprising a disruption of expression or activity of GR endogenous to the immune cells; (c) one or more integrin inhibitors; and (d) one or more TGF-beta inhibitors, wherein the two or more of (a), (b), (c), and (d) may or may not be in the same formulation. In specific cases, the composition comprises, consists essentially of, or consists of (a)(1) and (d); (a)(2) and (c); (a)(2) and (d); (b)(1), and (c); (b)(1) and (d); (b)(2) and (c); (b)(2) and (d); (c) and (d); (a)(1), (a)(2), (b), and (c); (a)(1), (a)(2), and (b); (a)(1), (a)(2), and (c); (a)(1), (b), and (c); (a)(2), (b), and (c); (b)(1), (b)(2), (c), and (d); (b)(1), (b)(2), and (c); (b)(1), (b)(2), and (d); (b)(1), (c), and (d); (a)(1), and (c); or (b)(2), (c), and (d). In specific cases, two or more of (a)(1), (a)(2), (b)(1), (b)(2), (c), and (d) are in the same formulation or are in different formulations.

The immune cells may be cord blood NK cells or are derived therefrom. In specific cases, the NK cells are expanded NK cells. In some cases, the one or more compounds that disrupt expression or activity of TGFBR2 and/or GR comprises nucleic acid, peptide, protein, small molecule, or a combination thereof. The nucleic acid may comprise siRNA, shRNA, anti-sense oligonucleotides, or guide RNA for CRISPR, merely as examples. In specific cases, the one or more integrin inhibitors comprises nucleic acid, peptide, protein (such as an antibody, including a monoclonal antibody), small molecule, or a combination thereof. The integrin inhibitors may target more than one integrin, and an example of an integrin inhibitor is cilengitide.

In specific embodiments, the one or more TGF-beta inhibitors comprises nucleic acid, peptide, protein (such as an antibody, including a monoclonal antibody), small molecule, or a combination thereof.

In particular embodiments, the immune cells are NK cells engineered to express a one or more CARs and/or one or more synthetic (non-native) T cell receptors. Either receptor may target a tumor antigen, including one associated with glioblastoma. In specific cases, the immune cells are NK cells that are engineered to express one or more heterologous cytokines.

Embodiments of the disclosure encompass methods of killing cancer cells in an individual, comprising the step of delivering to the individual a therapeutically effective amount of any composition(s) encompassed by the disclosure. In specific embodiments, the cancer cells are cancer stem cells and in other embodiments they are not; the cancer cells may be a mixture of cancer stem cells and cancer cells that are not cancer stem cells. The cancer may be of any kind, including a hematological cancer or a cancer that comprises one or more solid tumors. The cancer may be primary, metastatic, resistant to therapy, and so forth. The cancer may be of any stage. In specific cases, the cancer is glioblastoma, including glioblastoma comprising cancer stem cells.

Immune cells administered to an individual may or may not be allogeneic with respect to the individual. In specific cases, the immune cells are cord blood NK cells that are allogeneic with respect to the individual. The immune cells may or may not have been cryopreserved before the delivering step. In specific embodiments of the methods, the composition comprises an effective amount of combinations of (a)(1), (a)(2), (b)(1), (b)(2), (c), and (d) as noted above for the compositions. In specific embodiments, the combinations may be (a)(2) and (c); (b)(2) and (c); (a)(2) and (d); (b)(2) and (d); or (b) and (c). For any method of the disclosure, the individual may be delivered one or more additional cancer therapies, including at least surgery, radiation, chemotherapy, hormone therapy, immunotherapy, or a combination thereof.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawings.

FIGS. 1A-1F. GSCs express NK cell receptor ligands and are susceptible to NK cell cytotoxicity. (FIG. 1A) Healthy donor-derived NK cells were activated overnight with 5 ng/ml of IL-15 and co-cultured with GBM patient-derived GSCs (blue (middle) line), K562 (black (top) line) or healthy human astrocytes (red (bottom) line) targets for 4 hours at different effector:target ratios. The NK cell cytotoxic activity was measured by ⁵¹Cr-release assay (n=6). Error bars denote standard deviation. (FIG. 1B) Summary expression levels of 10 ligands for NK cell receptors on GSCs isolated from GBM patient samples or on healthy human astrocytes. The color scale of the heat map represents the relative expression of NK cell ligand on GSCs or human astrocytes ranging from blue (low expression) to red (high expression). The columns present the minimum to maximum and median expression for each receptor (GSC: n=6; Astrocytes: n=3); p=0.03 for ULP2/5/6, p<0.0001 for B7-H6 and p=0.02 for CD155. (FIG. 1C) Activated NK cells from healthy donors were co-cultured with GSCs for 18 hours in the presence or absence of blocking antibodies against the NK cell receptors NKG2D (blue line; bottom), DNAM (green line; middle), NKp30 (red line; second from bottom) or HLA class I (pink line; top). The NK cell cytotoxicity against GSCs targets was assessed by ⁵¹Cr-release assay (n=4). Error bars denote standard error of the mean. (FIGS. 1D-lE) viSNE plots (FIG. 1D) and a comparative heatmap of mass cytometry data (FIG. 1E) showing the expression of NK cell surface markers, transcription factors and cytotoxicity markers in HCNK (red), GBM patient PB-NK (green) and TiNK (blue). Heatmap column clustering is identified by FlowSOM analysis while each row reflects the expression level for an annotation for an individual patient. Color scale shows the expression level for each marker, with red representing higher expression and blue lower expression (n=3). (FIG. 1F) Violin plots showing the NK cell mRNA expression levels for individual genes between healthy control PB-NK cells (HC-NK; blue) and TiNK (red) using single-cell RNA sequencing. Markers associated with NK cell activation and cytotoxicity, NK cell inhibition and the TGF-β pathway are presented. P values were derived using unpaired t-test.

FIGS. 2A-2E. GSCs induce NK cell dysfunction. (FIG. 2A) Primary human GBM tumor infiltrating NK cells (TiNK) (red lines), paired peripheral blood NK cells (PB-NK) (blue lines) from the same GBM patient or peripheral blood NK cells from healthy control donor (HC-NK) (black lines) were co-cultured for 4 hours with K562 targets at different effector:target ratios and the cytotoxicity was determined by ⁵¹Cr release assay (n=8). Error bars denote standard deviation. (FIG. 2B) Box plots summarizing CD107a, IFN-γ, and TNF-α production by TiNKs, PB—NK or HC-NK cells after incubation with K652 targets for 5 hours at a 5:1 effector/target ratio. NK cells were identified as CD3-CD56+ lymphocytes (n=10). Error bars denote standard deviation. Paired t test was performed to determine statistical significance. (FIG. 2C) Box plots comparison of p-Smad⅔ expression in NK cells from healthy controls (HC-NK, white), PB-NK (red) and TiNKs (blue). Paired t test was performed to determine statistical significance (n=10). (FIG. 2D) Specific lysis (siCr release assay) of K562 cells by NK cells cultured alone or with GSCs at a 1:1 ratio for 48 h. Error bars denote standard deviation (p=0.03, n=10). (FIG. 2E) Box plots summarizing CD107a, IFN-γ, and TNF-α production by NK cells cultured either alone or with GSCs in a 1:1 ratio for 48 hours in response to K562 targets. Plots are gated on CD3-CD56+NK cells cultured alone or with GSCs (n=10).

FIGS. 3A-3H. GSC-induced NK cell dysfunction requires cell-to-cell contact. (FIG. 3A) Box plots summarize the mean fluorescence intensity (MFI) of p-Smad⅔ expression of NK cells cultured alone (NK alone) or co-cultured with GSCs in the presence or absence of the TGF-β receptor small molecule inhibitors LY2109761 (10 μM) and galunisertib (10 μM) for 12 hours. Paired t test was performed to determine statistical significance (n=4). (FIG. 3B) Specific lysis (⁵¹Cr release assay) of K562 (left) or GSCs (right) by purified healthy control NK cells after 48 hours of coculture with or without GSCs in a 1:1 ratio in the presence or absence of LY2109761 (10 μM) (K562: p=0.04; GSC: p=0.02) or galunisertib (10 μM) (K562: p=0.04; GSC: p=0.03) (n=4). Error bars denote standard deviation. (FIG. 3C) Specific lysis (siCr release assay) of K562 by TiNK after 24 hours of culture either alone or with galunisertib (10 μM) (n=3). (FIG. 3D) Box plots summarize the soluble TGF-β levels (pg/ml) by Elisa in the supernatant of NK cells and GSCs cultured either alone or together in the presence or absence of a transwell membrane for 48 hours (n=13). P values were derived using unpaired t-test. (FIG. 3E) Specific lysis (siCr release assay) of K562 by NK cells after 48 hours of co-culture with GSCs in a 1:1 ratio, either in direct contacted or separated by a transwell membrane (p=0.03) (n=7). (FIG. 3F) Box plots showing the MFI of p-Smad⅔ expression in healthy control NK cells cultured either alone, in direct contact with GSCs in the absence or presence of TGF-β blocking antibodies, or separated from GSCs by a transwell membrane (n=5). P values were derived using unpaired t-test. (FIG. 3G) Soluble TGF-β levels (pg/ml) in the supernatant of NK cells and GSCs cultured either alone (NK: blue line; GSC: black line) or together (red line) in the first 4 hours of co-culture was measured by ELISA (n=4). FIG. 3H, TGF-β mRNA fold change of NK cells and GSCs cultured either alone or together in the presence or absence of a transwell membrane for 48 hours was determined using qPCR (n=7).

FIGS. 4A-4G. αν integrins mediate TGF-β1 release by GSCs and GSC-induced NK cell dysfunction. (FIG. 4A) Box plots showing soluble TGF-β levels (pg/ml) in the supernatant of NK cells and GSCs cultured either alone or together in the presence or absence of the αν integrin small molecule inhibitor cilengitide (10 μM) for 48 hours was determined using ELISA (n=11). Error bars denote standard deviation. P values were derived using unpaired t-test. (FIG. 4B) Box plots showing the MFI of p-Smad⅔ expression on healthy control NK cells cultured either alone or with GSCs in the presence or absence of cilengitide (10 μM). P values were derived using paired t-test. (FIG. 4C) Specific lysis (siCr release assay) of K562 by NK cells cultured either alone or after co-culture with GSCs for 48 hrs in the presence or absence of cilengitide (10 μM) (p=0.05) (n=8). Error bars denote standard deviation. (FIGS. 4D-4E) Representative zebra plots (FIG. 4D) and summary box plots (FIG. 4E) of CD107, IFN-γ, and TNF-α production by NK cells in response to K562 cultured either alone or after 48 hrs of co-culture with GSCs at a 1:1 ratio with or without cilengitide (n=12). Inset numbers in (FIG. 4D) are the percentages of CD107a-, IFN-γ- or TNF-α-positive NK cells within the indicated regions. (FIG. 4F) Specific lysis (siCr release assay) of K562 targets by NK cells cultured either alone or with WT GSCs or with CD51 KO GSCs for 48 hrs at a 1:1 ratio (n=3). Error bars denote standard deviation. (FIG. 4G) Working hypothesis of GSCinduced NK cell suppression. Cell-cell contact between αν integrins (CD51) on GSCs with surface receptors such as CD9 and CD103 on NK cells mediate the release of TGF-β LAP through shear stress and the release MMP- 2/9 from GSCs. Free TGF-β is now able to bind its receptor on NK cells and induce immune suppression. Inhibition of the αν integrins on GSCs or knock out of the TGF-β receptor 2 on NK cells can prevent this chain of events, preserving the cytotoxic activity of NK cells and enabling them to efficiently target GSCs.

FIGS. 5A-5J. In vivo antitumor activity and NK cell function following TGF-β and αν integrin signaling inhibition in NSG GBM mouse model. (FIG. 5A) Timeline of in vivo experiments. GBM tumor implantation was performed at day 0 and ex vivo-expanded NK cells were administered intracranially at day 7 and then subsequently every 7 days for 11 weeks. Galunisertib was administered during this time period orally 5 times a week while cilengitide was administered intraperitoneally three times a week for the duration of the experiments. Bioluminescence imaging (BLI) was used to monitor the growth of firefly luciferase-labeled GBM tumor cells in NSG mice. (FIG. 5B) BLI was obtained from the six group of mice treated with GSC alone (untreated), GSC plus cilengitide, GSC plus galunisertib, GSC plus NK cells, GSC plus NK cells and cilengitide or GSC plus NK cells and galunisertib (4-5 mice per group) as described in panel FIG. (5A). (FIG. 5C) The plot summarizes the average radiance (BLI) data from our six groups of mice. Mice treated with NK cells together with cilengitide or galunisertib had a significantly lower tumor load by bioluminescence compared with untreated mice or mice treated with cilengitide alone (p<0.0001). Mice treated with NK cells plus galunisertib also had lower tumor load than those treated with galunisertib alone (p=0.04). (FIG. 5D) Kaplan-Meier plot showing the probability of survival for the groups of mice for each experimental group (5 mice per group). (FIGS. 5E-5F) viSNE plots (FIG. 5E) and a comparative heatmap (FIG. 5F) of mass cytometry data showing the expression of NK cell surface markers, transcription factors and cytotoxicity markers in WT NK cells, TGFPR2 KO NK cells, WT NK cells+ recombinant TGF-β or TGFPR2 KO NK cells+ recombinant TGF-β. Heatmap column clustering, generated by FlowSOM analysis Color scale, shows the expression level for each marker, with red representing higher expression and blue lower expression. On the left, the list of genes from top to bottom is CD16, CD8, LAG3, Granzyme A, Granzyme B, Perforin, DNAM, NKG2A, Ki67, 2B4, NKG2D, TIM3, CD96, NKP44, NKP46, T-Bet, CD39, NKP30, CD94, KLRG1, CD27, TIGIT, PANKIR, CD3z, CD2, CD69, CD25, TRAIL, NKG2C, CD9, CD103, Siglec 7, CD62L, Eomes, CCR6, and CD57 (FIG. 5G) Specific lysis of K562 targets over time by WT-NK (blue), TGFPR2 KO (black), WT-NK+ recombinant TGF-β (red) or TGFPR2 NK cells+ recombinant TGF-β (gray) as measured by Incucyte live imaging cell killing assay. (FIG. 5H) GBM tumor implantation was performed at day 0 and either WT or TGFPR2 KO NK cells were administered intracranially at day 7 and then subsequently every 4 weeks. Galunisertib was administered during this time period orally 5 times a week. (FIG. 5I) BLI was obtained from our three groups of mice: GSCs alone, GSCs plus WT NK cells and galunisertib or GSCs plus TGFPR2 KO NK cells (n=4 mice per group). (FIG. 5J) Plot summarizing the bioluminescence data from our four groups of mice from panel J. Mice treated with WT NK cells and galunisertib or TGFPR2 KO NK cells had a significantly lower tumor load by bioluminescence compared with untreated mice (p=0.001; p=0.0002 respectively). Error bars denote standard deviation.

FIGS. 6A-6C. GBM tumor infiltrating NK cells phenotype by flow cytometry. (FIGS. 6A-6B) Representative histograms and graph summary for the mean fluorescence intensity (MFI) or frequencies of NK cells expressing individual markers in GBM tumor infiltrating NK cells (TiNKs) vs. autologous peripheral blood (PB-NK) from the same GBM patient vs. peripheral blood from healthy controls (HC-NK). Error bars denote mean and standard deviation. P values were derived using paired (PB-NK vs. TiNK) and unpaired (HC-NK vs. TiNK) t-tests (n=28). (FIG. 6C) The color scale of the heatmap represents the relative expression for each marker ranging from blue (low expression) to red (high expression).

FIG. 7. GBM TiNK cells are dysfunctional. Representative zebra plot for CD107a, IFN-γ, and TNF-α production by TiNKs, PB—NK or HC-NK cells after incubation with K562 targets for 5 hours at a 5:1 effector: target ratio. Inset numbers are the percentages of CD107a-, IFN-γ- or TNF-α-positive NK cells within the gated populations.

FIGS. 8A-8B. TGF-β induces phosphorylation of Smad⅔ proteins in human NK cells by flow cytometry. (FIG. 8A) Representative histograms show the levels of p-Smad⅔ at baseline (red histogram) and after 30 minutes stimulation with 10 ng/ml of recombinant TGF-β in healthy control NK cells. (FIG. 8B) Representative histograms show the baseline levels of p-Smad⅔ in healthy control HC-NK cells (white histogram), GBM PB-NK cells (red histogram) and GBM TiNK cells (blue histogram).

FIGS. 9A-9C. GSCs but not healthy astrocytes induce NK cell dysfunction in vitro. (FIG. 9A) Specific lysis (siCr release assay) of K562 targets by NK cells cultured either alone (blue lines) or with healthy human astrocytes (red lines) in a 1:1 ratio for 48 hours (n=3). (FIG. 9B) Heathy donor NK cells were co-cultured with astrocytes for 48 hours at a 1:1 ratio. Representative zebra plots show their CD107a, IFN-γ, and TNF-α response to K562 targets. Effector:target ratio is 5:1. NK cells were gated on CD3-CD56+ lymphocytes (n=3). Inset numbers are the percentages of CD107a-, IFN-γ- or TNF-α-positive NK cells within the gated populations. (FIG. 9C) Heathy donor NK cells were co-cultured with GSCs for 48 hours at a 1:1 ratio. Representative zebra plots show their CD107a, IFN-γ, and TNF-α production in response to K562 targets. NK cells were defined as CD3-CD56+ lymphocytes (n=3). Inset numbers are the percentages of CD107a-, IFN-γ- or TNF-α-positive NK cells within the gated populations.

FIGS. 10A-10D. Blockade of TGF-β prevents GSC-induced NK cell dysfunction. (FIG. 10A) Representative zebra plots for CD107a, IFN-γ, and TNF-α production by NK cells in response to K562 targets after incubation with or without TGF-β blocking antibody (5 μg/ml). Effector:target ratio is 5:1. NK cells were gated on CD3-CD56+ lymphocytes. Inset numbers are the percentages of CD107a-, IFN-γ- or TNF-α-positive NK cells within the gated population. (FIG. 10B) Specific lysis (siCr release assay) of K562 targets by NK cells cultured either alone (blue lines) or co-cultured with GSCs for 48 hours at different effector:target ratios in the presence (black lines) or absence (red lines) of TGF-β blocking antibody (5 μg/ml) (p=0.04) (n=5). (FIGS. 10C-10D) Heathy donor NK cells were co-cultured with GSCs at a 1:1 ratio for 48 hours, in the presence or absence of TGF-β blocking antibody (5 μg/ml), Representative zebra plots and summary box plots show their CD107a, IFN-γ, and TNF-α response to K562 targets. Effector:target ratio is 5:1. NK cells were defined as CD3-CD56+ lymphocytes. Inset numbers are the percentages of CD107a-, IFN-γ- or TNF-α-positive NK cells within the gated population (n=5).

FIGS. 11A-11G. The TGF-β receptor kinase inhibitors Galunisertinib and LY2109761 prevent but do not reverse GSC-induced NK cell dysfunction in vitro. (FIG. 11A) NK cells were incubated with or without galunisertinib (10 μM) or LY2109761 (10 μM) for 48 hours. Representative zebra plots show their CD107a, IFN-γ, and TNF-α response to K562 targets. NK cells were gated on CD3-CD56+ lymphocytes. Inset numbers are the percentages of CD107a-, IFN-γ- or TNF-α-positive NK cells within the gated NK cell population. (FIGS. 11B-11C) NK cells were cultured either alone or with GSCs in a 1:1 ratio with or without LY2109761 or galunisertib for 48 hrs. Representative zebra plots and summary box plots show their CD107, IFN-γ, and TNF-α expression response to K562 (FIG. 11B) or GSC (FIG. 11C) targets. Effector:target ratio is 5:1. NK cells were defined as CD3-CD56+ lymphocytes. Inset numbers are the percentages of CD107a-, IFN-γ- or TNF-α-positive NK cells (n=7, n=4 respectively). (FIG. 11D) TiNK cells were cultured for 24 hours in media with 5 ng/ml IL-15 with or without galunisertib. Representative zebra plots and bar graph summary show their CD107, IFN-γ, and TNF-α response to K562 targets. Effector:target ratio is 5:1. NK cells were gated on CD3-CD56+ lymphocytes. Inset numbers are the percentages of CD107a-, IFN-γ- or TNF-α-positive NK cells within the indicated regions. (n=3). (FIG. 11E) TiNK cells and paired PB-NK cells from GBM patients were cultured in the presence or absence of galunisertib for 12 hours. The bar graphs summarize the mean fluorescence intensity (MFI) for their p-Smad⅔ expression. Paired t test was performed to determine statistical significance (n=3). (FIG. 11F) Specific lysis (⁵¹Cr release assay) of K562 targets by NK cells. After 48 hours of culture alone (blue lines) or with GSCs, healthy donor NK cells were either left with GSCs (red lines), or purified and re-suspended in media for another 48 hours (black lines). NK cells were then collected and used for ⁵¹Cr release assay (n=4). (FIG. 11G) Specific lysis (⁵¹Cr release assay) of K562 targets by NK cells. After 48 hours of culture alone (blue lines) or with GSCs, healthy donor NK cells were either left with GSCs (blue lines) or purified and cultured in SCGM media plus 5 ng/ml IL-15 with (black lines) or without galunisertib (10 μM) (gray lines) for 7 days. At the end of the culture period, their cytotoxicity was tested against k562 targets by ⁵¹Cr release assay (n=4).

FIG. 12. NK cells or astrocytes cultured either alone or together do not produce soluble TGF-β. Soluble TGF-β1 levels (pg/ml) measured by ELISA in the supernatant of NK cells and astrocytes cultured either alone or in direct contact at a 1:1 ratio for 48 hours (n=3).

FIG. 13. GSC-induced NK cell dysfunction is mediated through cell-cell contact. Healthy donor NK cells were cultured for 48 hours either alone, or with GSCs (1:1 ratio) in direct contact or with separation by a transwell membrane (n=6). Representative zebra and box plots summarize their CD107a, IFN-γ, and TNF-α response to K562. Effector:target ratio is 5:1. NK cells were gated on CD3-CD56+ lymphocytes. Inset numbers are the percentages of CD107a-, IFN-γ- or TNF-α-positive NK cells within the indicated regions.

FIGS. 14A-14B. TGF-β latency-associated peptide (LAP) is expressed on the surface of GSCs but not on NK cells. (FIG. 14A) Representative histograms show TGF-β LAP expression on the surface of GSCs and NK cells (blue histogram). Isotype control is shown in red. Inset numbers are the percentages of TGF-β LAP-positive GSCs (top) vs. NK cells (bottom) within the gated population. (FIG. 14B) Box plots summarize the TGF-β LAP surface expression on GSCs as measured by MFI (n=6). Error bars denote standard deviation. P values were derived using paired t-test.

FIGS. 15A-15G. MMP2 and MMP9 partially regulate TGF-β release by GSCs. (FIG. 15A) Box plots summarizing the levels of MMP2 and MMP9 (pg/ml) in the supernatant of NK cells and GSCs cultured either alone or together for 48 hours, either in direct cell contact or separated by a transwell membrane were measured using Luminex assay (n=10). P values were derived using paired t-test. (FIG. 15B) Box plots showing the MFI for MMP2 and MMP9 expression on NK cells or on GSCs cultured either alone or together in the presence or absence of g/ml of TGF-β blocking antibody (n=7). P values were derived using paired t-test. (FIG. 15C) Healthy donor NK cells were cultured with or without an MMP 2/9 inhibitor (1 μM) for 48 hours and their CD107a, IFN-γ, and TNF-α response to K562 targets was measured. NK cells were gated on CD3-CD56+ lymphocytes. Inset numbers are the percentages of CD107a-, IFN-γ- or TNF-α-positive NK cells within the indicated regions. (FIG. 15D) Healthy donor NK cells were cultured either alone (blue lines), or with GSCs at a 1:1 ratio with (black likes) or without (red lines) the MMP 2/9 inhibitor (1 μM) for 48 hrs. Their cytotoxicity (siCr release assay) was measured against K562 targets (p=0.04) (n=3). (FIGS. 15E-15F) Healthy donor NK cells were cultured either alone or with GSCs at a 1:1 ratio with or without the MMP 2/9 inhibitor for 48 hrs. Representative zebra plots and box plots summarize their CD107, IFN-γ, and TNF-α response to K562 targets (n=5). Effector:target ratio is 5:1. Inset numbers are the percentages of CD107a-, IFN-γ- or TNF-α-positive NK cells within the indicated regions. P values were derived using paired t-test. (FIG. 15G) Box plot show the expression of p-Smad⅔, as measured as MFI in NK cells in the presence or absence of GSCs, with or without the MMP 2/9 inhibitor (n=4). P values were derived using paired t-test.

FIGS. 16A-16C. Blocking of major NK cell receptors or their ligands has no impact on GSC-induced NK cell dysfunction. (FIGS. 16A-16C) Representative zebra plots for CD107a, IFN-γ, and TNF-α production by NK cells after culture with or without GSCs for 48 hours in the presence or absence of blocking antibodies against CD155/CD112, CD44, HLA-ABC and ILT-2. NK cells were gated on CD3-CD56+ lymphocytes. Inset numbers are the percentages of CD107a-IFN-γ- or TNF-α-positive NK cells within the indicated regions.

FIG. 17. CRISPR/Cas9 silencing of αν integrin (CD51) in GSCs. Representative histograms showing CD51 expression on the surface of wild type (WT) GSCs (white), GSCs treated with CRISPR Cas9 (GSCs Cas9 control; red) or GSCs after CD51 KO (blue).

FIGS. 18A-18E. CD9/CD103 expression on NK cells is induced by TGF-β and can be effectively silenced using CRISPR/Cas9 gene editing. (FIG. 18A) NK cells were cultured in SCGM, or in SCGM supplemented with 10 ng/ml TGF-β and/or 10 ng/ml IL-15, or with GSCs in a 1:1 ratio for 48 hours. After 48 hours, the cells were harvested and stained for surface expression of CD9. (FIG. 18B) Representative histograms showing the expression levels of CD9 (bottom) and CD103 (top) on the surface of NK cells following treatment with CRISPR Cas9 control (red), CRISPR Cas9 CD9 KO (bottom, blue) or CRISPR Cas9 CD103 KO (top, blue) as assessed by flow cytometry. (FIGS. 18C-18E) Representative zebra and box plots for CD107, IFN-γ, and TNF-α production by WT NK cells, CD9 KO NK cells, CD103 KO NK cells and CD9/CD103 double KO NK cells in response to K562 targets (n=6). Inset numbers are the percentages of CD107a-, IFN-γ- or TNF-α-positive NK cells within the indicated regions. P values were derived using paired t-test.

FIG. 19. NK cell therapy in combination with galunisertib or cilengitide eliminates glioblastoma in vivo. Photomicrographs showing severe infiltration and effacement of the cerebral gray matter by glioblastoma in an untreated control mouse in comparison to mice treated with combination therapy with NK cells and cilengitide or galunisertib, which shows no evidence of tumor. (H&E, 1.25× objective; 20× objective inset).

FIG. 20. Cilengitide treatment protects NK cells from TGF-β induced inhibitory phenotype in vivo. Heat map representation of the surface markers NKG2D, CD9, CD103, PD-1 and CD69. Once the mice were sacrificed, the brain tissue was processed and TiNKs were extracted from the GBM tumor. The cells we then stained for the indicated surface markers and analyzed using flow cytometry. The expression is presented as percentage of the cells expressing each marker within the total NK cell population.

FIGS. 21A-21B. CRISPR/Cas9 silencing of TGFPR2 in NK cells. (FIG. 21A) The TGFPR2 KO efficiency was determined by PCR. (FIG. 21B) Representative histograms showing abrogation of p-Smad⅔ signaling in TGFPR2 KO NK cells in response to treatment with exogenous TGF-β (10 ng/ml) for 45 mins compared to WT NK cells.

FIGS. 22A-22D. (FIGS. 22A-22C) Transcriptomic analysis of WT-NK and TGFPR2 KO before and after treatment with exogenous recombinant TGF-β (10 ng/ml) represented by heatmaps and volcano plots. No change in the gene expression profile was noted before and after treatment with recombinant TGF-β in TGFPR2 KO cells (n=3). (FIG. 22D) Specific lysis (siCr release assay) of K562 targets by WT-NK (blue), TGFPR2 KO NK cells (black) or TGFPR2 NK cells treated with recombinant TGF-β (10 ng/ml) for 48 hours prior to the assay (red and gray, respectively).

FIG. 23. Gating strategy for NK cell phenotyping using flow cytometry. Representative zebra plots for NK cell gating strategy. Inset numbers are the percentages of lymphocytes, single cells, live cells and NK cells within the indicated regions.

FIG. 24. GBM-infiltrating NK cells are highly dysfunctional. NK cells were ex vivo-selected from patient tumor (TiNK) and peripheral blood PB (GBM PB-NK). HC—NK refers to NK cells collected from healthy controls. PB healthy donor NK cells were used as controls. Multiparameter flow cytometry was used to analyze NK phenotype

FIGS. 25A-25B. Targeting the TGF-beta R2 gene by CRISPR gene editing. (FIG. 25A) Successful knockout of TGF-beta R2 in primary CB-NK cells using CRISPR/CAS9 technology (Cas9 plus gRNA targeting of exon 5 of TGF-beta R2) by PCR. (FIG. 25B) Examples of sequences of gRNA targeting by TGF-beta R2 gene (SEQ ID NOs:1-9).

FIGS. 26A-26B. Targeting of the glucocorticoid receptor (GR) and TGF-β R2 genes by CRISPR gene editing and anti-GBM response. (FIG. 26A) Successful knockout of GR and TGF-β R2 in primary CB-NK cells using CRISPR/CAS9 technology (Cas9 plus gRNA targeting of exon 2 of NR3C1 and exon 5 of TGF-β R2, respectively) by PCR. (FIG. 26B) CB-NK cell-mediated cytotoxicity of GSC spheroids was assessed in real time over a 24-hour period using an IncuCyte Live Cell Analysis System. Double KO NK cells exerted significantly greater killing of GSCs, even in the presence of 100 μM dexamethasone (DEX) (green and red lines; red is the top curve, green is the middle curve) compared to wild type (WT) NK cells (blue line that is the bottom curve) in the presence of DEX.

FIG. 27. TGF-beta was measured in supernatants from an NK:GBM co-cultured for 48 hours by ELISA. TGF-beta secretion was dependent on cell-cell contact with significantly greater amounts released when NK and GBM were cultured in direct contact (middle bar) compared to minimal secretion when NK cells were cultured either alone (left bar) or separated from GSCs by transwell (right bar).

FIGS. 28A-28C. CRISPR-Cas9 mediated deletion of TGFβR2 protects NK cells from the immunosuppressive effect of TGFβ. (FIG. 28A) viSNE plots of mass cytometry data in wild type (WT) NK cells and TGFβR2 KO NK cells cultured with or without exogenous TGF-β (10 ng/ml) demonstrating that the TGFβR2 KO construct protects the N cells from becoming dysfunctional. (FIG. 28B) Transcriptomic analysis of WT-NK and TGFβR2 KO before and after treatment with exogenous recombinant TGF-β (10 ng/ml) represented by volcano plots. No change in the gene expression profile was noted after treatment with recombinant TGF-β in TGFβR2 KO cells (n=3). (FIG. 28C) Specific lysis of K562 targets over time by WT-NK (blue; top line at least at 18-20 hrs), TGFβR2 KO (black), WT-NK cells+ recombinant TGF-β (red; middle line by itself) or TGFβR2 KO NK cells+ recombinant TGF-β (gray; lowest line in cluster) as measured by Incucyte live imaging cell killing assay; control K562 line is at the bottom.

FIGS. 29A-29C. CRISPR-Cas9 mediated deletion of the gene coding for the glucocorticoid receptor (GR) in primary human NK cells. (FIG. 29A) Schematic representation of CRISPR-Cas9 mediated NR3C1 targeting exon 2 of NR3C1 gene. (FIGS. 29B-29C) NR3C1 KO efficiency after electroporation with Cas9 alone (control), Cas9 complexed with one crRNA (crRNA 1 or crRNA 2) or Cas9 complexed with the combination of two crRNAs (crRNA 1+ crRNA 2) was determined by PCR at day 3 (FIG. 29B) or western blot at day 7 (FIG. 29C) after electroporation. crRNA1 in a 5′ to 3′ direction is CCTTGAGAAGCGACAGCCAGTGA (SEQ ID NO:19) and the complementary sequence is, in a 5′ to 3′ direction, TCACTGGCTGTCGGCTTCTCAAGG (SEQ ID NO:20). crRNA2 in a 5′ to 3′ direction is CCTGGCCAGACTGGCACCAACGG (SEQ ID NO:21) and the complementary sequence is, in a 5′ to 3′ direction, CCGTTGGTGCCAGTCTGGCCAGG (SEQ ID NO:22).

FIGS. 30A-30B. CRISPR-Cas9 Knockout of the genes encoding for TGF-β receptor 2 (TGFBR2) and the Glucocorticoid receptor (NR3C1) is feasible and efficient in CB derived NK cells. (FIG. 30A) Histograms showing mean fluorescent intensity (MFI) of p-SMAD 2/4. Following exposure to TGF-β, p-SMAD 2/4 is upregulated in WT NK cells, but not in TGFBR2 KO (alone or in conjunction with GR KO) NK cells. Absence of phosphorylation of p-SMAD 2/4 in the TGFBR2 KO conditions is a surrogate marker for an efficient deletion of the gene. (FIG. 30B) PCR gel electrophoresis showing efficient GR KO in CB derived NK cells performed alone or in conjunction with TGFBR2 KO, the primers are specific to exon 2 of NR3C1 (the gene encoding the GR protein).

FIGS. 31A-31C. GR KO protects from the immunosuppressive effects of Dexamethasone (In vitro cytotoxicity assay against GSC272). (FIGS. 31A-31C) CB-NK cell-mediated cytotoxicity of GSC spheroids using an IncuCyte Live Cell Analysis System. Incucyte cytotoxicity assay showing the killing of GSC272 over time among the different groups of NK cells (Wild type (WT), TGFBR2 KO, TGFBR2+GR KO) either untreated or treated with dexamethasone (Dexa). FIG. 31A. Graph showing the largest brightest green signal intensity (Caspase dye which correlates with tumor killing) over time among the different conditions. (FIG. 31B) Graph showing the red signal intensity (correlates with alive tumor) over time among the different conditions. GSC272 alone or with Dexa are used as controls. (FIG. 31C) Representative images from the Incucyte killing assay showing the green and red signal intensities among the different conditions. Double KO TGF-βR2-/GR-CB-NK cells exert significantly greater killing of GSCs even in the presence of 100 μM dexamethasone (DEX) (green and black lines) compared to wild type (WT) NK cells (blue line) in the presence of DEX.

FIGS. 32A-32E. In vivo antitumor activity and NK cell function following TGF-β signaling inhibition in NSG GBM mouse model. (FIG. 32A) A comparative heatmap of mass cytometry data showing the expression of NK cell surface markers, transcription factors and cytotoxicity markers in WT NK cells, TGFBR2 KO NK cells, WT NK cells+ recombinant TGF-βor TGFBR2 KO NK cells+ recombinant TGF-β. Heatmap column clustering, generated by FlowSOM analysis Color scale, shows the expression level for each marker, with red representing higher expression and blue lower expression. The list of genes is the same as for FIG. 5F. (FIG. 32B) Specific lysis of K562 targets over time by WT-NK (blue; top line at least for hours17-20), TGFBR2 KO (black), WT-NK+ recombinant TGF-β (red; middle line by itself) or TGFBR2 NK cells+ recombinant TGF-β (gray; lowest line in top cluster of lines) as measured by Incucyte live imaging cell killing assay; control K562 line is at the bottom. FIGS. 32C-32E, GBM tumor implantation was performed at day 0 and either WT or TGFβR2 KO NK cells were administered intracranially at day 7 and then subsequently every 4 weeks. Galunisertib was administered during this time period orally 5 times a week. (FIG. 32C) BLI was obtained from four groups of mice: GSCs alone, GSCs plus WT NK cells, GSC plus WT NK cells plus galunisertib, or GSCs plus TGFBR2 KO NK cells (n=4 mice per group). (FIG. 32D) Plot summarizing the bioluminescence data from our four groups of mice from panel C. Error bars denote standard deviation. The orange asterisks represent the statistical significance in bioluminescence in animals treated with TGFβR2 KO NK vs. untreated controls. The blue asterisks represent the statistical significance in bioluminescence in animals treated with WT NK cells plus Galunisertib vs. untreated controls. The green asterisks represent the statistical significance in bioluminescence in animals treated with WT NK cells vs. untreated controls, **p<0.01, ***p<0.001. (FIG. 32E) Kaplan-Meier plot showing probability of survival for mice in each experimental group. Animals treated with TGFBR2 KO NK cells had a significantly better survival compared to untreated tumor controls or mice treated with WT NK cells (p=0.009 and p=0.01, respectively).

### DETAILED DESCRIPTION

As used herein the specification, “a” or “an” may mean one or more. As used herein in the claim(s), when used in conjunction with the word “comprising”, the words “a” or “an” may mean one or more than one. As used herein “another” may mean at least a second or more. Still further, the terms “having”, “including”, “containing” and “comprising” are interchangeable and one of skill in the art is cognizant that these terms are open ended terms. In specific embodiments, aspects of the disclosure may “consist essentially of” or “consist of” one or more sequences of the disclosure, for example. Some embodiments of the invention may consist of or consist essentially of one or more elements, method steps, and/or methods of the disclosure. It is contemplated that any method or composition described herein can be implemented with respect to any other method or composition described herein. The scope of the present application is not intended to be limited to the particular embodiments of the process, machine, manufacture, composition of matter, means, methods and steps described in the specification. As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

As used herein, a “disruption” of a gene refers to the elimination or reduction of expression of one or more gene products encoded by the subject gene in a cell, compared to the level of expression of the gene product in the absence of the disruption. Exemplary gene products include mRNA and protein products encoded by the gene. Disruption in some cases is transient or reversible and in other cases is permanent. Disruption in some cases is of a functional or full length protein or mRNA, despite the fact that a truncated or non-functional product may be produced. In some embodiments herein, gene activity or function, as opposed to expression, is disrupted. Gene disruption is generally induced by artificial methods, i.e., by addition or introduction of a compound, molecule, complex, or composition, and/or by disruption of nucleic acid of or associated with the gene, such as at the DNA level. Exemplary methods for gene disruption include gene silencing, knockdown, knockout, and/or gene disruption techniques, such as gene editing. Examples include antisense technology, such as RNAi, siRNA, shRNA, and/or ribozymes, which generally result in transient reduction of expression, as well as gene editing techniques which result in targeted gene inactivation or disruption, e.g., by induction of breaks and/or homologous recombination. Examples include insertions, mutations, and deletions. The disruptions typically result in the repression and/or complete absence of expression of a normal or “wild type” product encoded by the gene. Exemplary of such gene disruptions are insertions, frameshift and missense mutations, deletions, knock-in, and knock-out of the gene or part of the gene, including deletions of the entire gene. Such disruptions can occur in the coding region, e.g., in one or more exons, resulting in the inability to produce a full-length product, functional product, or any product, such as by insertion of a stop codon. Such disruptions may also occur by disruptions in the promoter or enhancer or other region affecting activation of transcription, so as to prevent transcription of the gene. Gene disruptions include gene targeting, including targeted gene inactivation by homologous recombination.

The term “engineered” as used herein refers to an entity that is generated by the hand of man, including a cell, nucleic acid, polypeptide, vector, and so forth. In at least some cases, an engineered entity is synthetic and comprises elements that are not naturally present or configured in the manner in which it is utilized in the disclosure. In some cases, an engineered protein is a fusion of different components that are not found in the same configuration in nature.

The term “heterologous” as used herein refers to being derived from a different cell type or a different species than the recipient. In specific cases, it refers to a gene or protein that is synthetic and/or not from an NK cell. The term also refers to synthetically derived genes or gene constructs.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

The phrase “therapeutically effective amount” as used herein means that amount of a compound, material, or composition comprising a compound of the present disclosure that is effective for producing some desired therapeutic effect, e.g., treating (i.e., preventing and/or ameliorating) cancer in a subject, or inhibiting TGF-beta interactions with other molecules directly or indirectly, at a reasonable benefit/risk ratio applicable to any medical treatment. In one embodiment, the therapeutically effective amount is enough to reduce or eliminate at least one symptom. One of skill in the art recognizes that an amount may be considered therapeutically effective even if the cancer is not totally eradicated but improved partially. For example, the spread of the cancer may be halted or reduced, a side effect from the cancer may be partially reduced or completely eliminated, the onset of one or more symptoms may be delayed, the severity of one or more symptoms may be decreased, the life span of the subject may be increased, the subject may experience less pain, the quality of life of the subject may be improved, and so forth.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, a “mammal” is an appropriate subject for the method of the present invention. A mammal may be any member of the higher vertebrate class Mammalia, including humans; characterized by live birth, body hair, and mammary glands in the female that secrete milk for feeding the young. Additionally, mammals are characterized by their ability to maintain a constant body temperature despite changing climatic conditions. Examples of mammals are humans, cats, dogs, cows, mice, rats, horses, goats, sheep, and chimpanzees. Mammals may be referred to as “patients” or “subjects” or “individuals”.

The term “subject,” as used herein, generally refers to an individual in need of treatment, including for cancer. The subject can be any animal subject that is in need of treatment, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals. The subject can be a patient, e.g., have or be suspected of having a disease (that may be referred to as a medical condition), such as one or more cancers. The subject may be undergoing or having undergone cancer treatment. The subject may be asymptomatic. The term “individual” may be used interchangeably, in at least some embodiments. The “subject” or “individual”, as used herein, may or may not be housed in a medical facility and may be treated as an outpatient of a medical facility. The individual may be receiving one or more medical compositions via the internet. An individual may comprise any age of a human or non-human animal and therefore includes both adult and juveniles (e.g., children) and infants and includes in utero individuals. An individual may be of any gender or race.

As shown herein, tumors release TGF-beta after coming into direct contact with NK cells, and this interaction is mediated through integrins. If ex vivo-expanded NK cells are protected from the tumor microenvironment with administration of one or more TGF-beta inhibitors and/or one or more integrin inhibitors (simultaneously or not), then the NK antitumor cytotoxicity is maintained and is associated with marked enhancement of survival in animal models of GBM. In addition, and as one example only, upon using a novel Cas9 ribonucleoprotein (Cas9 RNP)-mediated gene editing approach to silence TGF-beta receptor 2 (TGF-betaR2), NK cells are protected from the immunosuppressive tumor microenvironment, and their in vitro and in vivo killing of the GBM cancer stem cells that give rise to recurrence is enhanced. In addition, it is shown herein that deletion of TGF-/βR2 and the glucocorticoid receptor gene (GR) in NK cells completely prevents GBM-induced dysfunction of healthy allogeneic NK cells and renders them resistant to the pro-apoptotic effect of corticosteroids. Based on these data, the disclosure provides a novel approach to immunotherapy (for GBM, for example) involving administration of NK cells of any kind in combination with one or more integrin inhibitors and/or one or more TGF-beta inhibitors and/or by targeting the TGF-betaR2 and/or GR genes of the NK cells being delivered by gene editing.

I. Compositions

Embodiments of the disclosure provide for one or more compositions for treatment or prevention of any cancer. The compositions may generally comprise one, two, three, or more active agents that provide therapy by themselves or are additive or synergistic with respect to one another. The different active agents may or may not be formulated together for storage, transport, and/or delivery.

In particular embodiments, compositions of the disclosure comprise one, two, or more of (a), (b), (c), and (d):

(a) one or both of (1) and (2):
(1) one or more compounds that disrupt expression or activity of transforming growth factor (TGF)-beta receptor 2 (TGFBR2);
(2) natural killer (NK) cells comprising a disruption of expression or activity of TGFBR2 endogenous to the immune cells;

(b) one or both of (1) and (2):
(1) one or more compounds that disrupt expression or activity of glucocorticoid receptor (GR);
(2) natural killer (NK) cells comprising a disruption of expression or activity of GR endogenous to the immune cells;

(c) one or more integrin inhibitors; and

(d) one or more TGF-beta inhibitors,

wherein two or more of (a), (b), (c), and (d) may or may not be in the same formulation.

With respect to the NK cells, they may be derived from one or more tissues, including at least cord blood, peripheral blood, bone marrow, hematopoietic stem cells, induced pluripotent stem cells, NK cell lines, or a mixture thereof. In a specific aspect, the NK cells are not derived from peripheral blood but are derived from cord blood or hematopoietic stem cells or induced pluripotent stem cells or NK cell lines. In some cases, NK cells already having a disruption of expression or activity of TGFBR2 endogenous to the NK cells are optionally derived from cord blood and the individual does not also receive (a)(1); (b); (c); and/or (d). In some cases, NK cells already having a disruption of expression or activity of GR endogenous to the NK cells are optionally derived from cord blood and the individual does not also receive (b)(1); (a); (c); and/or (d).

In specific embodiments, the composition comprises, consists essentially of, or consists of (a)(1) and (b); the composition comprises, consists essentially of, or consists of (a)(1) and (c); the composition comprises, consists essentially of, or consists of (a)(1) and (d); the composition comprises, consists essentially of, or consists of (a)(2) and (b); the composition comprises, consists essentially of, or consists of (a)(2) and (c); the composition comprises, consists essentially of, or consists of (a)(2) and (d); the composition comprises, consists essentially of, or consists of (b) and (c); the composition comprises, consists essentially of, or consists of (a)(1), (a)(2), and one or more of (b), (c), and (d); the composition comprises, consists essentially of, or consists of (a)(1), (a)(2), and (b); the composition comprises, consists essentially of, or consists of (a)(1), (a)(2), and (c); the composition comprises, consists essentially of, or consists of (a)(1), (a)(2), and (d); the composition comprises, consists essentially of, or consists of (a)(1), (b), (c), and (d); or the composition comprises, consists essentially of, or consists of (a)(2), (b), (c) and (d). In specific cases, two or more of (a)(1), (a)(2), (b), (c) and (d) are in the same formulation, or two or more of (a)(1), (a)(2), (b), (c) and (d) are in different formulations.

In specific embodiments, a therapy is synergistic or additive with respect to (a)(1) and any one or more of (a)(2), (b), (c) and (d); a therapy is synergistic or additive with respect to (a)(2) and any one or more of (a)(2), (b), (c) and (d); a therapy is synergistic or additive with respect to (a)(1) and (a)(2); and/or a therapy is synergistic or additive with respect to (b), (c) and/or (d), in some cases.

II. NK Cells

A. Gene Edited for TGF-beta R2 and/or GR

Embodiments of the disclosure include immunotherapy with immune cells including at least NK cells (although in some embodiments the immune cells are T cells, NK T cells, iNKT cells, gamma delta T cells, cytokine-induced killer (CIK) cells, B cells, dendritic cells, macrophages, etc.). The immunotherapy comprises (1) NK cells that themselves are engineered to be more effective at cancer treatment than NK cells that are not so engineered; and/or (2) one or more agents that are utilized in combination with NK cells of any kind to be more effective at cancer treatment than in the absence of the NK cells.

In some embodiments, the NK cells are engineered to have reduction or elimination of expression of endogenous TGF-beta R2 (also referred to herein as TGFBR2) and/or activity of the expressed protein, and such engineering may occur by any suitable means. Thus, the NK cells may be gene edited, and the gene editing may occur by any means. The gene editing may or may not be transient; in specific cases the gene editing is permanent.

In some embodiments, the NK cells are engineered to have reduction or elimination of expression of endogenous GR and/or activity of the expressed protein, and such engineering may occur by any suitable means. Thus, the NK cells may be gene edited, and the gene editing may occur by any means. The gene editing may or may not be transient; in specific cases the gene editing is permanent. In some embodiment glucocorticoid receptor gene (GR) in NK cells completely prevents GBM-induced dysfunction of healthy allogeneic NK cells and renders them resistant to the pro-apoptotic effect of corticosteroids.

In some embodiments, the gene disruption is carried out by effecting a disruption in the gene, such as a knock-out, insertion, missense or frameshift mutation, including biallelic frameshift mutation, deletion of all or part of the gene, e.g., one or more exon or portion therefore, and/or knock-in, as some examples. In certain cases, the disruption can be affected be sequence-specific or targeted nucleases, including DNA-binding targeted nucleases such as zinc finger nucleases (ZFN) and transcription activator-like effector nucleases (TALENs), and RNA-guided nucleases such as a CRISPR-associated nuclease (Cas), specifically designed to be targeted to the sequence of the TGF-betaR2 gene or a portion thereof or specifically designed to be targeted to the sequence of the GR gene or a portion thereof.

In some embodiments, TGF-betaR2 gene and/or GR gene disruption is performed by induction of one or more double-stranded breaks and/or one or more single-stranded breaks in the gene, including in a targeted manner. In some embodiments, the double-stranded or single-stranded breaks are made by a nuclease, e.g., an endonuclease, such as a gene-targeted nuclease. In some aspects, the breaks are induced in the coding region of the gene, e.g., in an exon. For example, in some embodiments, the induction occurs near the N-terminal portion of the coding region, e.g., in the first exon, in the second exon, or in a subsequent exon.

In some embodiments, gene disruption is achieved using antisense techniques, including by RNA interference (RNAi), short interfering RNA (siRNA), short hairpin (shRNA), and/or ribozymes are used to selectively suppress or repress expression of the gene. siRNA technology is RNAi that employs a double-stranded RNA molecule having a sequence homologous with the nucleotide sequence of mRNA that is transcribed from the gene, and a sequence complementary with the nucleotide sequence. siRNA generally is homologous/complementary with one region of mRNA that is transcribed from the gene, or may be siRNA including a plurality of RNA molecules that are homologous/complementary with different regions. In some aspects, the siRNA is comprised in a polycistronic construct.

In some embodiments, the disruption is achieved using a DNA-targeting molecule, such as a DNA-binding protein or DNA-binding nucleic acid, or complex, compound, or composition, containing the same, which specifically binds to or hybridizes to the TGF-beta R2 gene. In some embodiments, the DNA-targeting molecule comprises a DNA-binding domain, e.g., a zinc finger protein (ZFP) DNA-binding domain, a transcription activator-like protein (TAL) or TAL effector (TALE) DNA-binding domain, a clustered regularly interspaced short palindromic repeats (CRISPR) DNA-binding domain, or a DNA-binding domain from a meganuclease. Zinc finger, TALE, and CRISPR system binding domains can be engineered to bind to a predetermined nucleotide sequence, for example via engineering (altering one or more amino acids) of the recognition helix region of a naturally occurring zinc finger or TALE protein. Engineered DNA binding proteins (zinc fingers or TALEs) are proteins that are non-naturally occurring. Rational criteria for design include application of substitution rules and computerized algorithms for processing information in a database storing information of existing ZFP and/or TALE designs and binding data.

In cases where gene alteration is carried out by induction of one or more double-stranded breaks and/or one or more single-stranded breaks in the gene, the double-stranded or single-stranded breaks may undergo repair via a cellular repair process, such as by non-homologous end-joining (NHEJ) or homology-directed repair (HDR). In some aspects, the repair process is error-prone and results in disruption of the gene, such as a frameshift mutation, e.g., biallelic frameshift mutation, which can result in complete knockout of the gene. For example, in some aspects, the disruption comprises inducing a deletion, mutation, and/or insertion. In some embodiments, the disruption results in the presence of an early stop codon. In some aspects, the presence of an insertion, deletion, translocation, frameshift mutation, and/or a premature stop codon results in disruption of the expression, activity, and/or function of the gene.

In some embodiments, the alteration is carried out using one or more DNA-binding nucleic acids, such as alteration via an RNA-guided endonuclease (RGEN). For example, the alteration can be carried out using clustered regularly interspaced short palindromic repeats (CRISPR) and CRISPR-associated (Cas) proteins. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type III CRISPR system, e.g., derived from a particular organism comprising an endogenous CRISPR system, such as Streptococcus pyogenes.

The NK cell may be introduced to a guide RNA and CRISPR enzyme, or mRNA encoding the CRISPR enzyme. For CRISPR-mediated disruption, the guide RNA and endonuclease may be introduced to the NK cells by any means known in the art to allow delivery inside cells or subcellular compartments of agents/chemicals and molecules (proteins and nucleic acids) can be used including liposomal delivery means, polymeric carriers, chemical carriers, lipoplexes, polyplexes, dendrimers, nanoparticles, emulsion, natural endocytosis or phagocytose pathway as non-limiting examples, as well as physical methods such as electroporation. In specific aspects, electroporation is used to introduce the guide RNA and endonuclease, or nucleic acid encoding the endonuclease.

In one exemplary method, the method for CRISPR knockout of multiple genes may comprise isolation of immune cells, such as NK cells, from cord blood or peripheral blood or hematopoietic cells or induced pluripotent stem cells, or NK cell lines, or a mixture thereof. The NK cells may be isolated and seeded on culture plates with irradiated feeder cells, such as at a 1:2 ratio. The cells can then be electroporated with gRNA and Cas9 in the presence of IL-2, such as at a concentration of 200 IU/mL. The media may be changed every other day. After 1-3 days, the NK cells are isolated to remove the feeder cells and can then be transduced with a CAR construct. The NK cells may then be subjected to a second CRISPR Cas9 knockout for additional gene(s). After the electroporation, the NK cells may be seeded with feeder cells, such as for 5-9 days.

In some aspects, a Cas nuclease and gRNA (including a fusion of crRNA specific for the target sequence and fixed tracrRNA) are introduced into the NK cell. In general, target sites at the 5′ end of the gRNA target the Cas nuclease to the target site, e.g., the TGF-beta R2 gene, using complementary base pairing. The target site may be selected based on its location immediately 5′ of a protospacer adjacent motif (PAM) sequence, such as typically NGG, or NAG. In this respect, the gRNA is targeted to the desired sequence by modifying the first 20, 19, 18, 17, 16, 15, 14, 14, 12, 11, or 10 nucleotides of the guide RNA to correspond to the target DNA sequence. In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

The CRISPR system can induce double stranded breaks (DSBs) at the target site, followed by disruptions or alterations as discussed herein. In other embodiments, Cas9 variants, deemed “nickases,” are used to nick a single strand at the target site. Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor or activator, to affect gene expression.

The target sequence may comprise any polynucleotide, such as DNA or RNA polynucleotides. The target sequence may be located in the nucleus or cytoplasm of the cell, such as within an organelle of the cell. Generally, a sequence or template that may be used for recombination into the targeted locus comprising the target sequences is referred to as an “editing template” or “editing polynucleotide” or “editing sequence”. In some aspects, an exogenous template polynucleotide may be referred to as an editing template. In some aspects, the recombination is homologous recombination.

Typically, in the context of an endogenous CRISPR system, formation of the CRISPR complex (comprising the guide sequence hybridized to the target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. The tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of the CRISPR complex, such as by hybridization along at least a portion of the tracr sequence to all or a portion of a tracr mate sequence that is operably linked to the guide sequence. The tracr sequence has sufficient complementarity to a tracr mate sequence to hybridize and participate in formation of the CRISPR complex, such as at least 50%, 60%, 70%, 80%, 90%, 95% or 99% of sequence complementarity along the length of the tracr mate sequence when optimally aligned.

One or more vectors driving expression of one or more elements of the CRISPR system can be introduced into the cell such that expression of the elements of the CRISPR system direct formation of the CRISPR complex at one or more target sites. Components can also be delivered to cells as proteins and/or RNA. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. The vector may comprise one or more insertion sites, such as a restriction endonuclease recognition sequence (also referred to as a “cloning site”). In some embodiments, one or more insertion sites are located upstream and/or downstream of one or more sequence elements of one or more vectors. When multiple different guide sequences are used, a single expression construct may be used to target CRISPR activity to multiple different, corresponding target sequences within a cell.

A vector may comprise a regulatory element operably linked to an enzyme-coding sequence encoding the CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csxl2), Cas10, Csy1, Csy2, Csy3, Cse1, Cse2, Csc1, Csc2, Csa5, Csn2, Csm2, Csm3, Csm4, Csm5, Csm6, Cmrl, Cmr3, Cmr4, Cmr5, Cmr6, Csb1, Csb2, Csb3, Csxl7, Csxl4, Csxl0, Csxl6, CsaX, Csx3, Csxl, Csxl5, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2.

The CRISPR enzyme can be Cas9 (e.g., from S. pyogenes or S. pneumonia). The CRISPR enzyme can direct cleavage of one or both strands at the location of a target sequence, such as within the target sequence and/or within the complement of the target sequence. The vector can encode a CRISPR enzyme that is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce NHEJ or HDR.

In some embodiments, an enzyme coding sequence encoding the CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

In general, a guide sequence is any polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97%, 99%, or more.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), Clustal W, Clustal X, BLAT, Novoalign (Novocraft Technologies, ELAND (Illumina, San Diego, Calif.), SOAP (available at soap.genomics.org.cn), and Maq (available at maq.sourceforge.net).

The CRISPR enzyme may be part of a fusion protein comprising one or more heterologous protein domains. A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions. Additional domains that may form part of a fusion protein comprising a CRISPR enzyme are described in US 20110059502, incorporated herein by reference.

In some embodiments, the alteration of the expression, activity, and/or function of the TGF-beta R2 is carried out by disrupting the corresponding gene. In some aspects, the gene is modified so that its expression is reduced by at least at or about 20, 30, or 40%, generally at least at or about 50, 60, 70, 80, 90, 91, 92, 93, 94, 95, 96, 97, 98, 99, or 100% as compared to the expression in the absence of the gene modification or in the absence of the components introduced to effect the modification.

B. NK Cell Modifications Other Than TGF-beta R2 and/or GR Disruption

In some cases, the NK cells for immunotherapy have additional modifications than gene editing of TGF-beta R2 and/or GR. In specific embodiments, the NK cells are modified to express one or more engineered non-natural receptors, such as a chimeric antigen receptor (CAR), a T cell receptor, a cytokine receptor, a chemokine receptor, homing receptor, or a combination thereof. The NK cells may alternatively or additionally be engineered to express one or more heterologous cytokines and/or engineered to increase expression of one or more endogenous cytokines of any kind. The NK cells may be modified to have a suicide gene.

In cases where particular NK cells are in need of expressing one or more heterologous genes or expression constructs, the one or more genes or expression constructs may or may not be transfected into the NK cells on the same vector. The vector may be of any kind including integrating or non-integrating. The vector may or may not be viral. The vector may comprise nanoparticles, plasmids, transposons, adenoviral vectors, adenoviral-associated vectors, retroviral vectors, lentiviral vectors, and so forth.

1. Engineered Receptors

In specific embodiments, the NK cells of the immunotherapy comprise one or more engineered receptors that are non-natural to the NK cells, such as chimeric antigen receptors (CAR), synthetic (non-native) T cell receptors, cytokine receptors, chemokine receptors, homing receptors, or a combination thereof. Such engineered receptors may themselves be fusion proteins of two or more components.

In some cases the engineered receptor targets any particular ligand, such as an antigen, including a cancer antigen (including a tumor antigen). The cancer antigens may be of any kind, including those associated with a particular cancer to be treated and that is desired to be targeted for specific elimination of the cancer. The engineered receptors (and the NK cells themselves) may be tailored for a specific cancer. In specific cases, the antigen is an antigen associated with glioblastoma, including EGFR, EGFRvIII, HER2, CMV, CD70, chlorotoxin, IL12Roa2, MICA/B/ULBP, etc.

For those engineered receptors that comprise an antigen binding domain, the antigen binding domain may comprise at least one scFv, for example, and it may comprise 2-3 scFvs. Antigenic molecules may come from infectious agents, auto-/self-antigens, tumor-/cancer-associated antigens, or tumor neoantigens, for example. Examples of antigens that may be targeted include but are not limited to antigens expressed on B-cells; antigens expressed on carcinomas, sarcomas, lymphomas, leukemia, germ cell tumors, and/or blastomas; antigens expressed on various immune cells; and antigens expressed on cells associated with various hematologic diseases, autoimmune diseases, and/or inflammatory diseases. Examples of specific antigens to target include CD70, CD38, HLA-G, BCMA, CD19, CD5, CD99, CD33, CLL1, CD123, 4-1BB, 5T4, adenocarcinoma antigen, alpha-fetoprotein, BAFF, B-lymphoma cell, C242 antigen, CA-125, carbonic anhydrase 9 (CA-IX), C-MET, CCR4, CD152, CD20, CD200, CD22, CD221, CD23 (IgE receptor), CD28, CD30 (TNFRSF8), CD33, CD4, CD38, CD40, CD44 v6, CD51, CD52, CD56, CD74, CD80, CEA, CNTO888, CTLA-4, DRS, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgG1, L1-CAM, IL-13, IL-6, insulin-like growth factor I receptor, integrin-a5bl, integrinavb3, MORAb-009, MS4A1, MUC1, mucin CanAg, Nglycolylneuraminic acid, NPC-1C, PDGF-Ralpha, PDL192, phosphatidylserine, prostatic carcinoma cells, RANKL, RON, ROR1, SCH 900105, SDC1, SLAMF7, TAG-72, tenascin C, TGF β2, TGF-β, TRAIL-R1, TRAIL-R2, tumor antigen CTAA16.88, VEGF-A, VEGFR-1, VEGFR2, vimentin, and combinations thereof. Any antigen receptor that may be utilized in methods and compositions of the disclosure may target any one of the above-referenced antigens, or one or more others, and such an antigen receptor may be a CAR or a TCR. The same cells for therapy may utilize both a CAR and a TCR, in specific embodiments.

The CAR may be first generation, second generation, or third or subsequent generation, for example. The CAR may or may not be bispecific to two or more different antigens. The CAR may comprise one or more co-stimulatory domains. Each co-stimulatory domain may comprise the costimulatory domain of any one or more of, for example, members of the TNFR superfamily, CD28, CD137 (4-1BB), CD134 (OX40), Dap10, DAP12, 2B4, NKG2D, CD27, CD2, CD5, ICAM-1, LFA-1 (CD11a/CD18), Lck, TNFR-I, TNFR-II, Fas, CD30, CD40 or combinations thereof, for example. In specific embodiments, the CAR comprises CD3zeta. In certain embodiments, the CAR lacks one or more specific costimulatory domains; for example, the CAR may lack 4-1BB and/or CD28.

In particular embodiments, the CAR polypeptide in the NK cells comprises an extracellular spacer domain that links the antigen binding domain and the transmembrane domain. Extracellular spacer domains may include, but are not limited to, Fc fragments of antibodies or fragments or derivatives thereof, hinge regions of antibodies or fragments or derivatives thereof, CH2 regions of antibodies, CH3 regions antibodies, artificial spacer sequences or combinations thereof. Examples of extracellular spacer domains include but are not limited to CD8-alpha hinge, CD28, artificial spacers made of polypeptides such as Gly3, or CH1, CH3 domains of IgGs (such as human IgG1 or IgG4). In specific cases, the extracellular spacer domain may comprise (i) a hinge, CH2 and CH3 regions of IgG4, (ii) a hinge region of IgG4, (iii) a hinge and CH2 of IgG4, (iv) a hinge region of CD8-alpha, (v) a hinge, CH2 and CH3 regions of IgG1, (vi) a hinge region of IgG1 or (vii) a hinge and CH2 of IgG1, (viii) a hinge region of CD28, or a combination thereof.

In specific embodiments, the hinge is from IgG1 and in certain aspects the CAR polypeptide comprises a particular IgG1 hinge amino acid sequence or is encoded by a particular IgG1 hinge nucleic acid sequence.

2. Cytokines

In some embodiments, the NK cells are engineered to express one or more heterologous cytokines and/or are engineered to upregulate normal expression of one or more heterologous cytokines. Although in some cases any cytokine may be utilized, in specific cases the cytokine is IL-7, IL-2, IL-15, IL-12, IL-18, IL-21, GMCSF, or a combination thereof. The NK cells may or may not be transduced or transfected for one or more cytokines on the same vector as other genes.

3. Suicide Genes

In some cases, the NK cells are modified to produce one or more agents other than heterologous cytokines, engineered receptors, and so forth. In specific embodiments, the NK cells are engineered to harbor one or more suicide genes, and the term “suicide gene” as used herein is defined as a gene which, upon administration of a prodrug, effects transition of a gene product to a compound which kills its host cell. In some cases, the NK cell therapy may be subject to utilization of one or more suicide genes of any kind when an individual receiving the NK cell therapy and/or having received the NK cell therapy shows one or more symptoms of one or more adverse events, such as cytokine release syndrome, neurotoxicity, anaphylaxis/allergy, and/or on-target/off tumor toxicities (as examples) or is considered at risk for having the one or more symptoms, including imminently. The use of the suicide gene may be part of a planned protocol for a therapy or may be used only upon a recognized need for its use. In some cases the cell therapy is terminated by use of agent(s) that targets the suicide gene or a gene product therefrom because the therapy is no longer required.

Examples of suicide genes include engineered nonsecretable (including membrane bound) tumor necrosis factor (TNF)-alpha mutant polypeptides (see PCT/US19/62009, which is incorporated by reference herein in its entirety), and they may be affected by delivery of an antibody that binds the TNF-alpha mutant. Examples of suicide gene/prodrug combinations that may be used are Herpes Simplex Virus-thymidine kinase (HSV-tk) and ganciclovir, acyclovir, or FIAU; oxidoreductase and cycloheximide; cytosine deaminase and 5-fluorocytosine; thymidine kinase thymidilate kinase (Tdk::Tmk) and AZT; and deoxycytidine kinase and cytosine arabinoside. The E. coli purine nucleoside phosphorylase, a so-called suicide gene that converts the prodrug 6-methylpurine deoxyriboside to toxic purine 6-methylpurine, may be utilized. Other suicide genes include CD20, CD52, inducible caspase 9, purine nucleoside phosphorylase (PNP), Cytochrome p450 enzymes (CYP), Carboxypeptidases (CP), Carboxylesterase (CE), Nitroreductase (NTR), Guanine Ribosyltransferase (XGRTP), Glycosidase enzymes, Methionine-α,γ-lyase (MET), and Thymidine phosphorylase (TP), as examples.

III. Integrin Inhibitors

In particular embodiments, one or more integrin inhibitors are utilized with other therapies encompassed herein, such as at least NK cells, including NK cells gene edited for reduction in expression or activity for TGF-beta R2 and/or GR, for example.

Integrins are activatable adhesion and signaling molecules, and inhibitors encompassed herein may target any integrin. Integrins are comprised of α (alpha) and β (beta) molecules, and any inhibitor for use in the methods and compositions of the disclosure may target one of α or β or a combination thereof. In some cases, the integrin inhibitor targets a101, a2p1, α3β1, α4β1, α5β1, α6β1, α7β1, αLβ2, αMβ2, αIIbβ3, αVβ1, αVβ, αVβ5, αVβ6, αVβ8, and/or α6β4 specifically. The integrin inhibitor may target the ligand or the receptor. The inhibition may occur by direct interaction with the ligand and/or the receptor.

One or more integrin inhibitors may comprise, consist of, or consist essentially of nucleic acid, peptide, protein, small molecule, or a combination thereof. In some cases, the integrin inhibitor is a small molecule or an antibody of any kind, including a monoclonal antibody. In some cases, the integrin inhibitor is nucleic acid that is siRNA, shRNA, anti-sense oligonucleotides, or guide RNA for CRISPR to knockdown or knockout one or more integrin genes.

In some cases, specific integrin inhibitor(s) are utilized. In a specific example, cilengitide is used, although alternatives may be employed. In specific cases, cilengitide is not used. In some embodiments, one or more of the following integrin inhibitors are utilized in methods and compositions of the disclosure: (1) cilengitide; (2) Abciximab (3) Eptifibatide; (4) Tirofiban; (5) Natalizumab; (6) Vedolizumab; (7) etaracizumab; (8) abegrin; (9) CNTO95; (10) ATN-161; (11) vipegitide; (12) MK0429; (13) E7820; (14) Vitaxin; (15) 5247; (16) PSK1404; (17) S137; (18); HYD-1; (19) abituzumab; (20) Intetumumab; (21) RGD-containing linear or cyclic peptide, including at least Cyclo(RGDyK); (22) Lifitegrast; (23) Leukadherin-1; (24) A 205804, a selective inhibitor of E-selectin and ICAM-1 expression; (25) A 286982, an inhibitor of the LFA-1/ICAM-1 interaction; (26) ATN 161, α5β1 integrin receptor antagonist; (27) BIO 1211, selective α4β1 (VLA-4) inhibitor; (28) BIO 5192, selective inhibitor of integrin α4β1 (VLA-4); (29) BMS 688521, inhibitor of the LFA-1/ICAM interaction; (30) BOP, dual α9β1/α4β1 integrin inhibitor; preferentially mobilizes HSCs; (31) BTT 3033, selective inhibitor of integrin α2β1; (32) E 7820, α2 integrin inhibitor and anti-angiogenic; (33) Echistatin, α1 isoform, αVβ3 and glycoprotein IIb/IIIa (integrin aIIbβ3) inhibitor; (34) GR 144053 trihydrochloride, glycoprotein IIb/IIIa (integrin aIIbβ3) receptor antagonist and antithrombotic; (35) MNS, gycoprotein IIb/IIIa (aIIbβ3) inhibitor and also inhibits Src and Syk; (36) Obtustatin, selective α1β1 inhibitor; (37) P11, antagonist of ανβ3-vitronectin interaction and antiangiogenic; (38) R-BC154, high affinity fluorescent α4β1/α9β1 inhibitor and mobilizes HSCs; (39) RGDS peptide, an integrin binding sequence and inhibits integrin receptor function; (40) TC-I 15, α2β1 inhibitor and displays antithrombotic activity in vivo; and (41) TCS 2314, α4β1 (VLA-4) antagonist.

In some embodiments, cilengitide is used in combination with one or both of TGF-beta inhibitor galunisertib and TGF-beta R2 KO NK cells and/or GR KO NK cells.

In cases wherein integrin inhibitors are utilized, they may be formulated in a composition with one or more TGF-beta inhibitors and/or TGF-beta R2 KO NK cells and/or GR KO NK cells.

In specific cases, one or more integrin inhibitors are provided to the individual for the directed purpose of treating cancer with the one or more integrin inhibitors.

IV. TGF-beta Inhibitors

Transforming growth factor beta (TGF-β) is a multifunctional cytokine belonging to the transforming growth factor superfamily that comprises three different mammalian isoforms (TGF-beta1; TGF-beta2; and TGF-beta3) and many other signaling proteins. In particular embodiments, one or more TGF-beta inhibitors are utilized with other therapies encompassed herein, such as at least NK cells, including NK cells gene edited for knocking down or knocking out TGF-beta R2, for example.

In the present disclosure “a TGF-beta inhibitor” is understood as any compound capable of preventing signal transmission caused by the interaction between TGF-beta and its receptor. The TGF-beta inhibitor(s) may target the ligand or the receptor. The inhibition may occur by direct interaction with the ligand and/or the receptor.

One or more TGF-beta inhibitors may comprise, consist of, or consist essentially of nucleic acid, peptide, protein, small molecule, or a combination thereof. In some cases, the integrin inhibitor is a small molecule or an antibody of any kind, including a monoclonal antibody.

In some cases, the TGF-beta inhibitor is nucleic acid that is siRNA, shRNA, anti-sense oligonucleotides, or guide RNA for CRISPR to knockdown or knockout the TGF-beta gene; one example of a nucleic acid is Trabedersen.

In some embodiments, the active agent is a TGF-beta pathway inhibitor. In some embodiment, the active agent is a TGF-beta inhibitor that is trafficked by macrophage to a site of inflammation or degeneration where the inhibitor can renormalize overly activated TGF-beta pathway. In another embodiment, the active agent is a TGF-beta inhibitor that is delivered to peripheral macrophages and/or monocytes, for example in a cell-specific manner.

Examples of TGF-beta inhibitors include Galunisertib; Fresolimumab; Lucanix; Vigil; Trabedersen; Belagenpumatucel-L; gemogenovatucel-T; SB525334; SB431542; ITD-1; LY2109761; LY 3200882; SB505124; Pirfenidone; GW788388; LY364947; LY2157299; RepSox; SD-208; IN 1130; SM 16; A 77-01; AZ 12799734; Lovastin; A83-01; LY 364947; SD-208; SJN 2511; Soluble proteins that naturally bind to and inhibit TGF-beta (one or more of LAP, decorin, fibromodulin, lumican, endoglin, alpha2-macroglobulin); or a combination thereof. In some cases, the TGF-beta inhibitor is an inhibitor of ALK4, ALK5 and/or ALK7. For example, the TGF-beta inhibitor may bind to and directly inhibit ALK4, ALK5 and/or ALK7.

In some embodiments, galunisertibe is used in combination with one or both of integrin inhibitor cilengitide and TGF-beta R2 KO NK cells and/or GR KO NK cells.

In cases wherein TGF-beta inhibitors are utilized, they may be formulated in a composition with one or more integrin inhibitors and/or TGF-beta R2 KO NK cells and/or GR KO NK cells.

In specific cases, one or more TGF-beta inhibitors are provided to the individual for the directed purpose of treating cancer with the one or more TGF-beta inhibitors.

V. Methods of the Disclosure

Embodiments of the disclosure include improved immunotherapy methods of treating or preventing any kind of cancer, including hematological malignancies or solid tumors. Hematological malignancies include at least cancers of the bone marrow, T or B cell malignancies, leukemias, lymphomas, blastomas, myelomas, and the like. Specific examples include at least Acute myeloid leukemia, B-cell acute lymphoblastic leukemia, T-cell acute lymphoblastic leukemia, Myelodysplastic syndromes, Chronic lymphocytic leukemia/small lymphocytic lymphoma, Follicular lymphoma, Lymphoplasmacytic lymphoma, Diffuse large B-cell lymphoma, Mantle cell lymphoma, Hairy cell leukemia, Plasma cell myeloma or multiple myeloma, Mature T/NK neoplasms, and so forth. Examples of solid tumors include tumors of the brain, lung, breast, prostate, pancreas, stomach, anus, head and neck, bone, skin, liver, kidney, thyroid, testes, ovary, endometrium, gall bladder, peritoneum, cervix, colon, rectum, vulva, spleen, a combination thereof, and so forth.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia; chronic lymphocytic leukemia (CLL); acute lymphoblastic leukemia (ALL); acute myeloid leukemia (AML); and chronic myeloblastic leukemia.

Methods of the disclosure encompass immunotherapies including adoptive cellular therapy, with immune cells such as NK cells (whether expanded or not) for treating cancer, where the immunotherapies are improved to allow greater efficacy for the immunotherapy by inhibiting released inhibitory TGF-beta (such as from cancer cells) or inhibiting associated interactions, such as the relationship between TGF-beta and integrins, or inhibiting the ability of TGF-beta to bind to the immune cells (by knocking out its receptor in NK cells). Such modification of NK cells and therapies that may be used with the modified NK cells allows for greater efficacy in cancer treatment. In specific embodiments, it allows for killing of cancer stem cells of any kind, including of the brain, blood, breast, colon, ovary, pancreas, prostate, melanoma, head and neck, cervix, uterus, lung, mesothelioma, stomach, esophageal, rectal, lymphoma, multiple myeloma, or non-melanoma skin cancer, for example.

In some embodiments, the present disclosure provides methods for immunotherapy comprising administering an effective amount of NK cells of the present disclosure, wherein the NK cells are particularly modified and/or the individual is also treated with integrin inhibitor(s) and/or TGF-beta inhibitor(s). In one embodiment, a medical disease or disorder is treated at least by particular NK cells that elicit an immune response in the recipient. In certain embodiments of the present disclosure, any cancer, such as glioblastoma, is treated by transfer of a specific NK cell population that elicits an immune response. Provided herein are methods for treating or delaying progression of cancer in an individual comprising administering to the individual an effective amount of an antigen-specific cell therapy when gene-modified NK cells comprise molecules, such as receptors, that can target a desired antigen.

In specific embodiments, an individual is treated for glioblastoma, and the methods of the disclosure in methods for treating glioblastoma comprise the steps of killing brain cancer stem cells, including without killing astrocytes.

In particular embodiments of the present disclosure, an effective amount of NK cells are delivered to an individual in need thereof, such as an individual that has cancer of any kind. The cells then enhance the individual's immune system to attack the cancer cells. In some cases, the individual is provided with one or more doses of the NK cells. In cases where the individual is provided with two or more doses of the NK cells, the duration between the administrations should be sufficient to allow time for propagation in the individual, and in specific embodiments the duration between doses may be 1, 2, 3, 4, 5, 6, 7, or more days, or 1, 2, 3, 4, or more weeks, 1, 2, 3, 4, 5, 6, 7, 8, 9, 10, 11, 12, or more months, 1, 2, 3, 4, 5, 6, 7, 8, 9, 10, or more years, and so forth. Successive doses may or may not be identical in amount to one another. In some cases, the successive doses decrease over time or increase over time.

Methods of the disclosure encompass delivery of an effective amount of compositions comprising (or consisting of or consisting essentially of) two or more of (a), (b), (c), and (d):

(a) one or both of (1) and (2):
(1) one or more compounds that disrupt expression or activity of transforming growth factor (TGF)-beta receptor 2 (TGFBR2);
(2) natural killer (NK) cells comprising a disruption of expression or activity of TGFBR2 endogenous to the immune cells;

(b) one or both of (1) and (2):
(1) one or more compounds that disrupt expression or activity of glucocorticoid receptor (GR);
(2) natural killer (NK) cells comprising a disruption of expression or activity of GR endogenous to the immune cells;

(c) one or more integrin inhibitors; and

(d) one or more TGF-beta inhibitors, wherein two or more of (a), (b), (c), and (d) may or may not be in the same formulation.

In cases wherein the two or more of (a), (b), (c), and (d) may or may not be in the same formulation, the two or more components may be delivered at separate times or at substantially the same time. In cases wherein an order of delivery of the two or more components is desired, the order may be of any kind so long as the delivery is therapeutically effective. In specific embodiments, delivery of (a) precedes delivery of (b), (c) and/or (d). In specific embodiments, delivery of (b) precedes delivery of (a), (c), and/or (d). In specific embodiments, delivery of (c) precedes delivery of (a), (b), and/or (d). In specific embodiments, delivery of (d) precedes delivery of (a), (b), and/or (c). In specific embodiments, (b) and (c) are delivered prior to delivery of any NK cells of any kind, including TGFbeta R2 KO NK cells and/or GR KO NK cells.

In particular embodiments, any NK cells encompassed herein are utilized in an off-the-shelf manner wherein the NK cells are gene modified as described herein and stored until needed for use. At such time, the NK cells may be further modified, including, for example, to customize a therapy for an individual in need thereof. In specific examples, the NK cells are then customized to express one or more engineered antigen receptors that comprise antigen binding domain(s) that target an antigen on cancer cells of the individual. In such cases, the individual may also receive an effective amount of one or more integrin inhibitors and/or one or more TGF-beta inhibitors.

VI. Pharmaceutical Compositions

Pharmaceutical compositions of the present disclosure comprise an effective amount of one or more integrin inhibitors, one or more TGF-beta inhibitors, TGFbeta R2 KO NK cells and/or GR KO NK cells (and/or reagents to generate same ex vivo or in vivo) dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that comprises one or more integrin inhibitors, one or more TGF-beta inhibitors, TGFbeta R2 KO and/or GR KO NK cells (and/or reagents to generate same ex vivo or in vivo) will be known to those of skill in the art in light of the present disclosure, as exemplified by Remington: The Science and Practice of Pharmacy, 21^{st} Ed. Lippincott Williams and Wilkins, 2005, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The pharmaceutical compositions may comprise different types of carriers depending on whether it is to be administered in solid, liquid or aerosol form, and whether it need to be sterile for such routes of administration as injection. The presently disclosed compositions can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The one or more integrin inhibitors, one or more TGF-beta inhibitors, TGFbeta R2 KO and/or GR KO NK cells (and/or reagents to generate same ex vivo or in vivo) may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present disclosure, the compositions of the present disclosure suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present disclosure, the composition is combined with the carrier in any convenient and practical manner, i.e., by solution, suspension, emulsification, admixture, encapsulation, absorption and the like. Such procedures are routine for those skilled in the art.

In a specific embodiment of the present disclosure, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the present disclosure may concern the use of a pharmaceutical lipid vehicle compositions that include one or more integrin inhibitors, one or more TGF-beta inhibitors, TGFbeta R2 KO NK cells, and/or GR KO NK cells (and/or reagents to generate same ex vivo or in vivo), and optionally an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds that contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the one or more integrin inhibitors, one or more TGF-beta inhibitors, TGF-beta R2 KO NK cells and/or GR KO NK cells (and/or reagents to generate same ex vivo or in vivo) may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present disclosure administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

A. Alimentary Compositions and Formulations

In preferred embodiments of the present disclosure, the one or more integrin inhibitors, one or more TGF-beta inhibitors, TGFbeta R2 KO NK cells and/or GR KO NK cells (and/or reagents to generate same ex vivo or in vivo) are formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792, 451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the compositions of the present disclosure may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

Additional formulations that are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

B. Parenteral Compositions and Formulations

In further embodiments, compositions may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,613,308; 5,466,468; 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

C. Miscellaneous Pharmaceutical Compositions and Formulations

In other preferred embodiments of the invention, the active compound one or more integrin inhibitors, one or more TGF-beta inhibitors, TGFbeta R2 KO NK cells and/or GR KO NK cells (and/or reagents to generate same ex vivo or in vivo) may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-solubly based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

VII. Combination Therapies

In certain embodiments, the compositions and methods of the present embodiments involve a cancer therapy that is additional to the compositions comprising one or more integrin inhibitors; one or more TGF-beta inhibitors; and/or TGF-beta R2 KO NK cells and/or GR KO NK cells. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, hormone therapy, or a combination of the foregoing. The additional therapy may be in the form of adjuvant or neoadjuvant therapy.

In some embodiments, the additional therapy is the administration of small molecule enzymatic inhibitor(s) or anti-metastatic agent(s). In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemopreventative agent(s). The additional therapy may be one or more of the chemotherapeutic agents known in the art.

An immune cell therapy (in addition to the NK cell therapy of the disclosure) may be administered before, during, after, or in various combinations relative to an additional cancer therapy, such as immune checkpoint therapy. The administrations may be in intervals ranging from concurrently to minutes to days to weeks. In embodiments where the immune cell therapy is provided to a patient separately from the composition(s) of the disclosure, one would generally ensure that a significant period of time did not expire between the time of each delivery, such that the two compounds would still be able to exert an advantageously combined effect on the patient. In such instances, it is contemplated that one may provide a patient with the immunotherapy therapy and the disclosed compositions within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

Administration of any compound or cell therapy of the present embodiments to a patient will follow general protocols for the administration of such compounds, taking into account the toxicity, if any, of the agents. Therefore, in some embodiments there is a step of monitoring toxicity that is attributable to combination therapy.

A. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclophosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammalI and calicheamicin omegaIl); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine,plicomycin, gemcitabien, navelbine, farnesyl-protein tansferase inhibitors, transplatinum, and pharmaceutically acceptable salts, acids, or derivatives of any of the above.

B. Radiotherapy

Other factors that cause DNA damage and have been used extensively include what are commonly known as 7-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

C. Immunotherapy

The skilled artisan will understand that additional immunotherapies (outside of the disclosed NK cell therapy) may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells other than those having knockdown or knockout of TGF-beta R2.

Antibody-drug conjugates have emerged as a breakthrough approach to the development of cancer therapeutics. Antibody-drug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. There are currently more than 30 ADC drug candidates in various stages of clinical trials for cancer treatment (Leal et al., 2014). As antibody engineering and linker-payload optimization are becoming more and more mature, the discovery and development of new ADCs are increasingly dependent on the identification and validation of new targets that are suitable to this approach and the generation of targeting MAbs. Two criteria for ADC targets are upregulated/high levels of expression in tumor cells and robust internalization.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons of any kind, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

D. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

E. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

VIII. Kits of the Disclosure

Any of the compositions described herein may be comprised in a kit. In a non-limiting example, one or more integrin inhibitors, one or more TGF-beta inhibitors, TGFbeta R2 KO NK cells, GR KO NK cells (and/or reagents to generate same), and these may be comprised in suitable container means in a kit of the present disclosure.

The compositions of the kits may be packaged either in aqueous media or in lyophilized form. The container means of the kits will generally include at least one vial, test tube, flask, bottle, syringe or other container means, into which one or more components may be placed, and preferably, suitably aliquoted. Where there are more than one component in the kit, the kit also may generally contain a second, third or other additional container into which the additional components may be separately placed. However, various combinations of components may be comprised in a vial. The kits of the present invention also will typically include a means for containing the one or more integrin inhibitors, one or more TGF-beta inhibitors, TGFbeta R2 KO NK cells (and/or reagents to generate same), GR KO NK cells, and any other reagent containers, in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly envisioned. The compositions may also be formulated into a syringeable composition. In which case, the container means may itself be a syringe, pipette, and/or other such like apparatus, from which the formulation may be applied to an infected area of the body, injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent. It is envisioned that the solvent may also be provided in another container means.

Irrespective of the number and/or type of containers, the kits of the disclosure may also comprise, and/or be packaged with, an instrument for assisting with the injection/administration and/or placement of the ultimate composition within the body of an animal. Such an instrument may be a syringe, pipette, forceps, and/or any such medically approved delivery vehicle. In some embodiments, reagents or apparatuses or containers are included in the kit for ex vivo use.

### EXAMPLES

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Natural Killer Cell Immunotherapy for Cancer Treatment

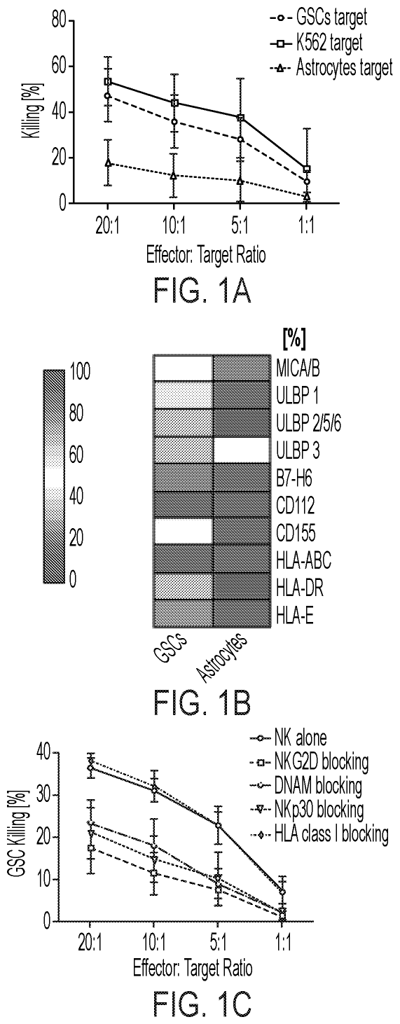

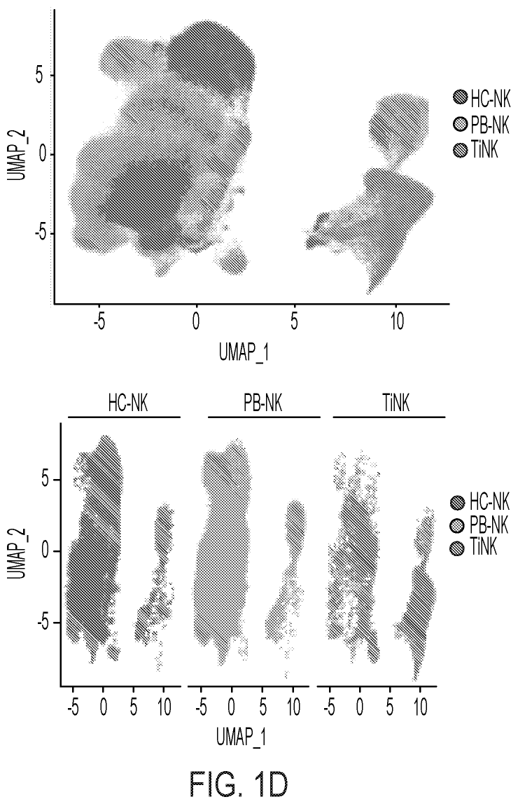

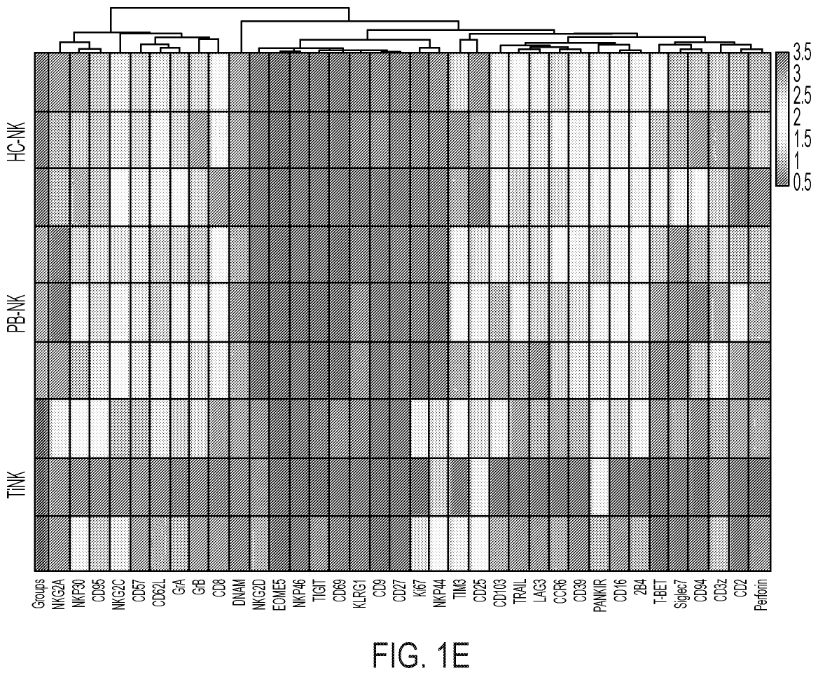

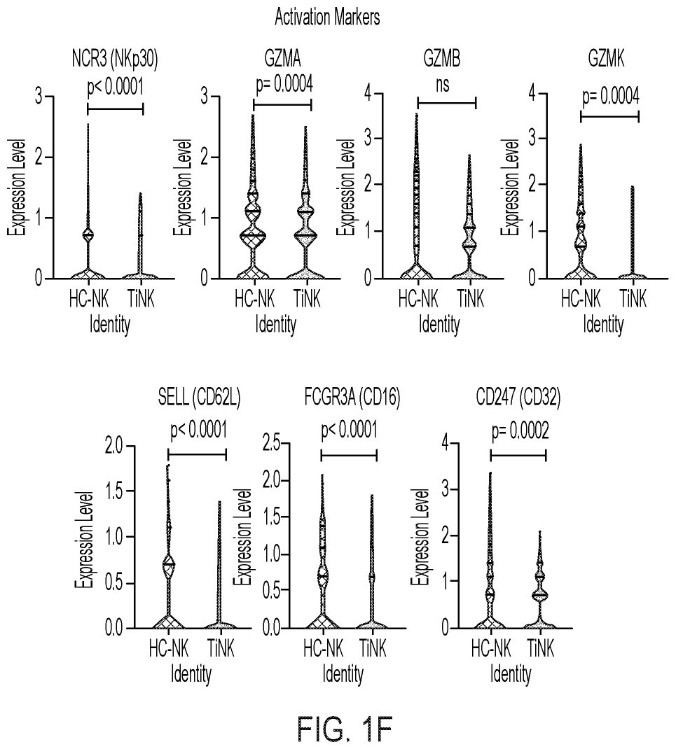

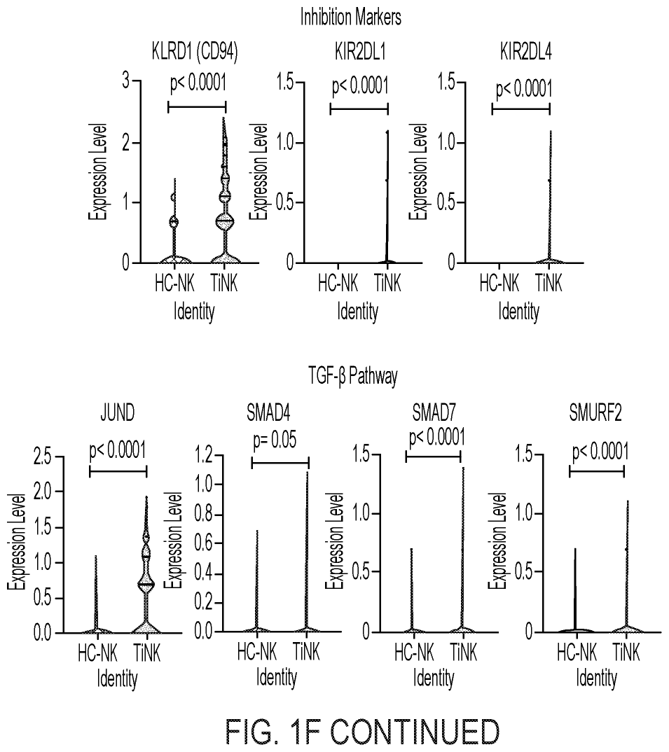

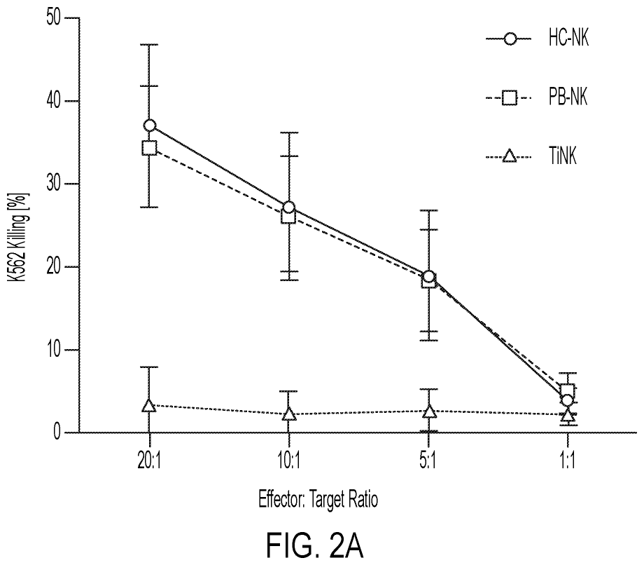

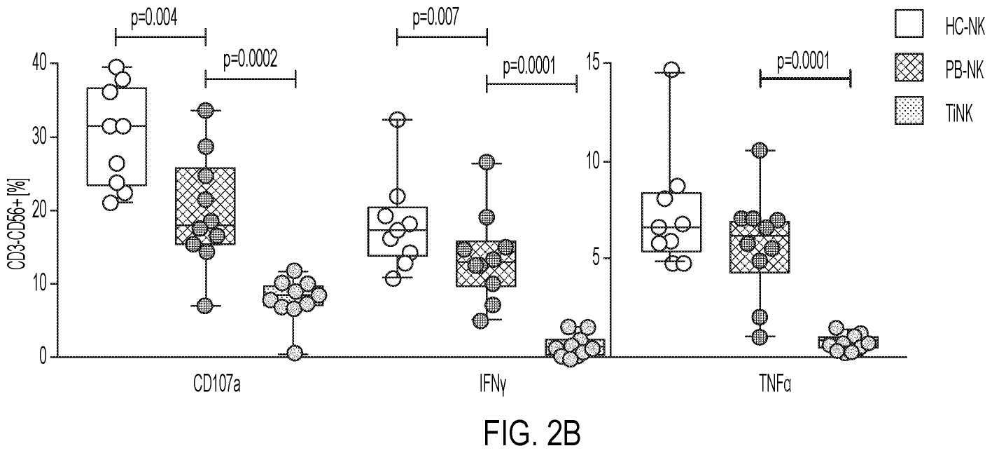

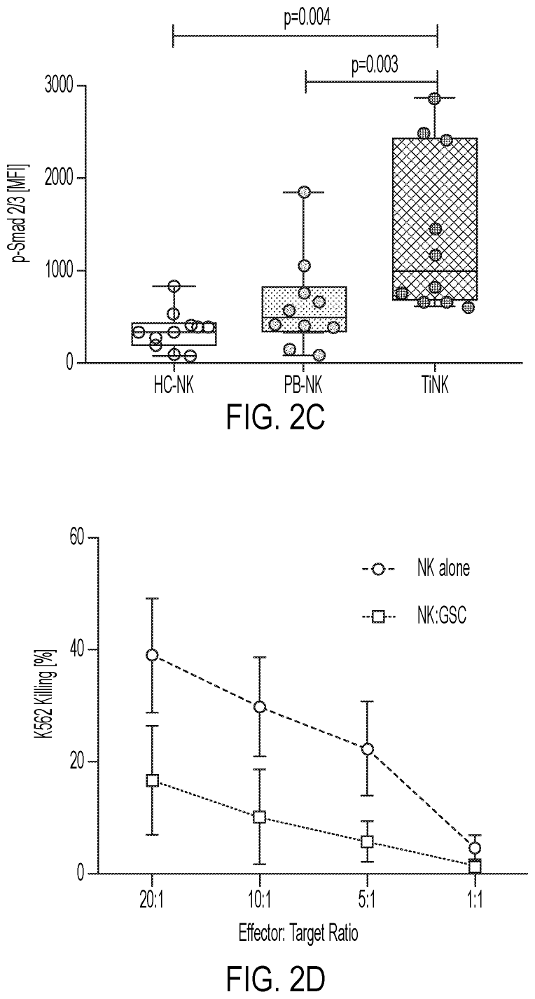

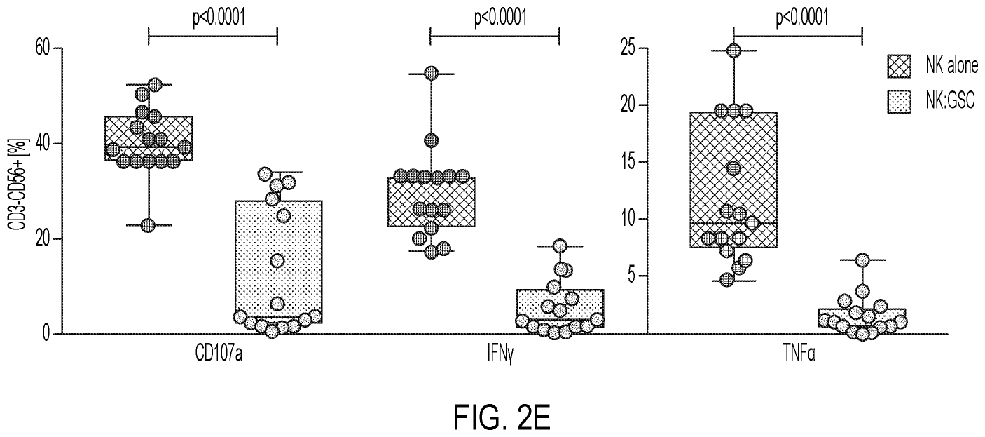

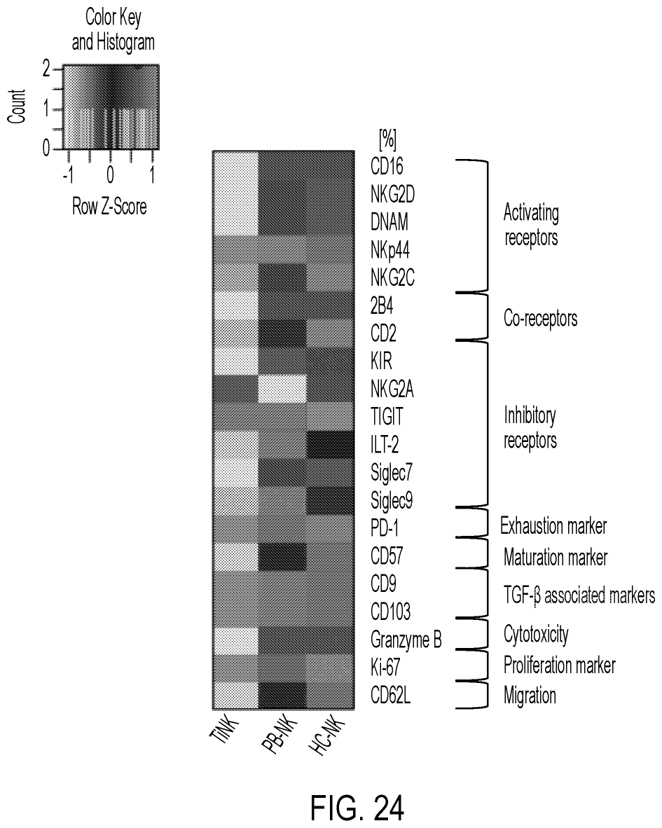

The present example concerns the use of NK cells for treatment of cancer of any kind, including at least glioblastoma. NK cells can kill patient-derived glioblastoma stem cell lines (GCSs) but not normal astrocytes (FIGS. 1A-1B). FIG. 2 demonstrates that GBM-infiltrating NK cells are highly dysfunctional. In FIG. 24, NK cells were ex vivo-selected from patient tumor (TiNK) and peripheral blood PB (GBM PB-NK). PB healthy donor NK cells were used as controls. Multiparameter flow cytometry was used to analyze NK phenotype. In FIG. 2A, NK effector function against K562 targets was assessed using ⁵¹Cr release assay.

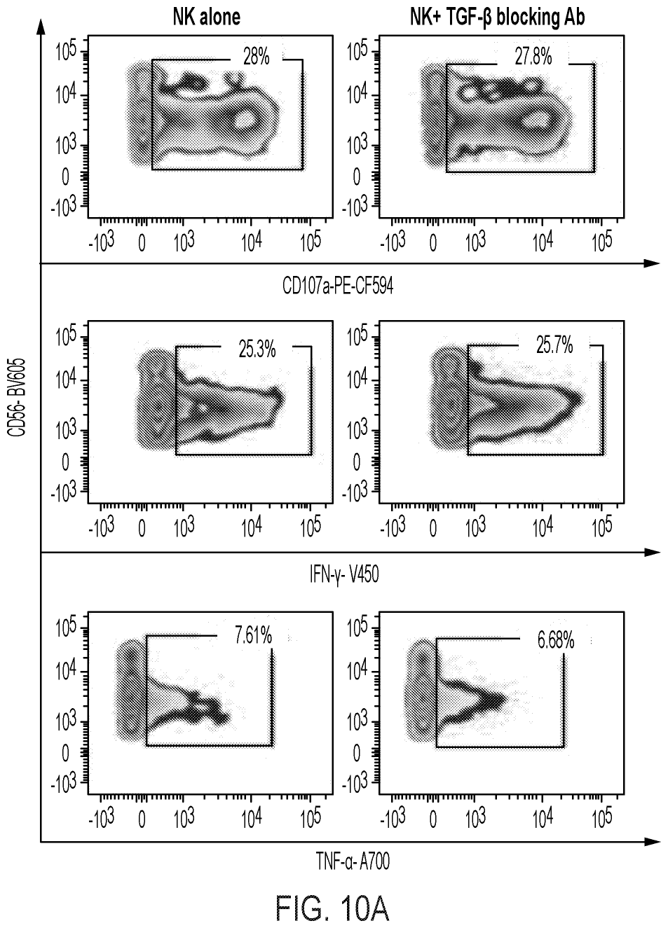

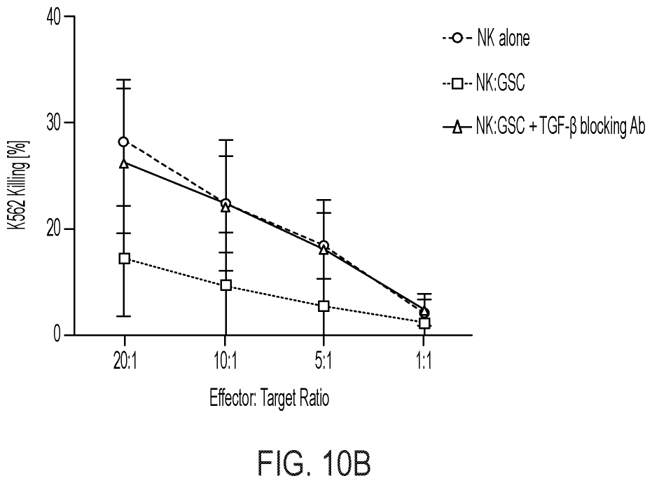

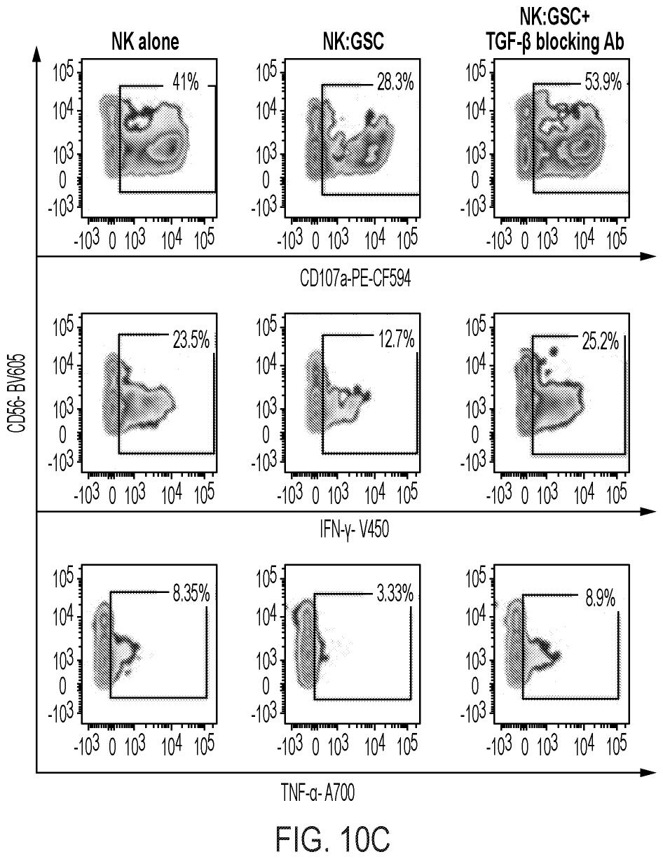

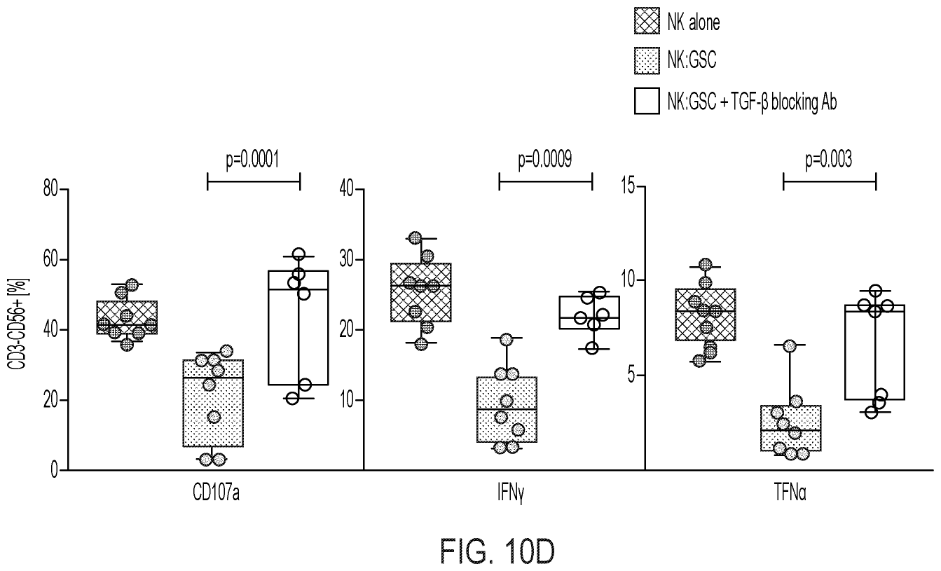

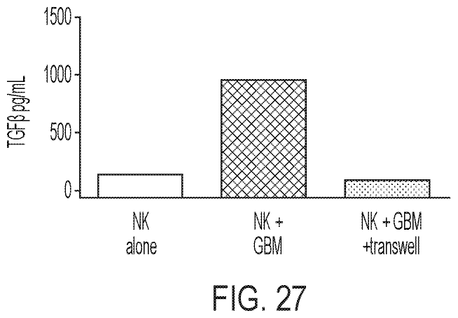

It was determined that GBM-induced NK cell dysfunction is mediated through TGF-beta and cell-cell contact. In FIG. 10B, healthy NK cells were co-cultured with GSC in 1:1 ratio for 48 hours, in the presence or absence of TGF-beta blocking antibody. Culture with TGF-beta blocking antibody prevented GBM-induced NK dysfunction as measured by cytotoxicity in response to the K562 targets; bottom line is NK cells+ GCS coculture. In FIG. 27, TGF-beta was measured in supernatants from an NK:GBM co-cultured for 48 hours by ELISA. TGF-beta secretion was dependent on cell-cell contact with significantly greater amounts released when NK and GBM were cultured in direct contact compared to minimal secretion when NK cells were cultured either alone or separated from GSCs by transwell.

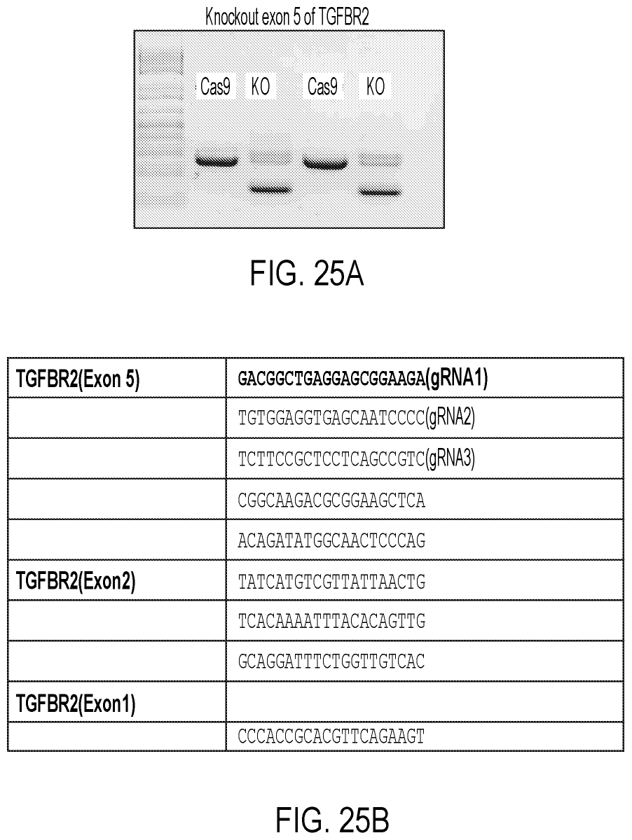

NK cells were tested for the ability to be reduced in expression of TGF-beta R2, in particular FIG. 25 shows targeting the TGF-beta R2 gene by CRISPR gene editing. FIG. 25A shows successful knockout of TGF-beta R2 in primary CB-NK cells using CRISPR/CAS9 technology (Cas9 plus gRNA targeting of exon 5 of TGF-beta R2) by PCR. FIG. 25B provides examples of sequences of gRNA targeting by TGF-beta R2 gene.

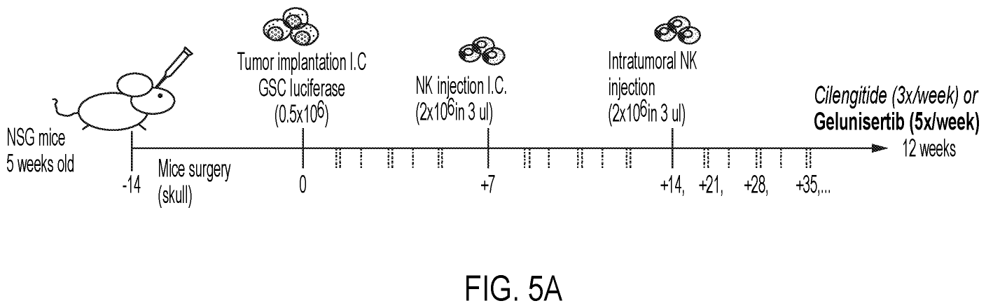

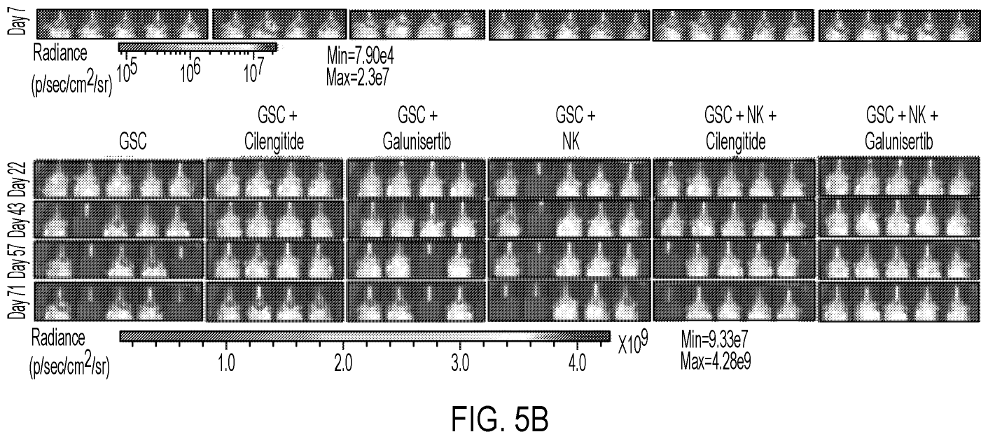

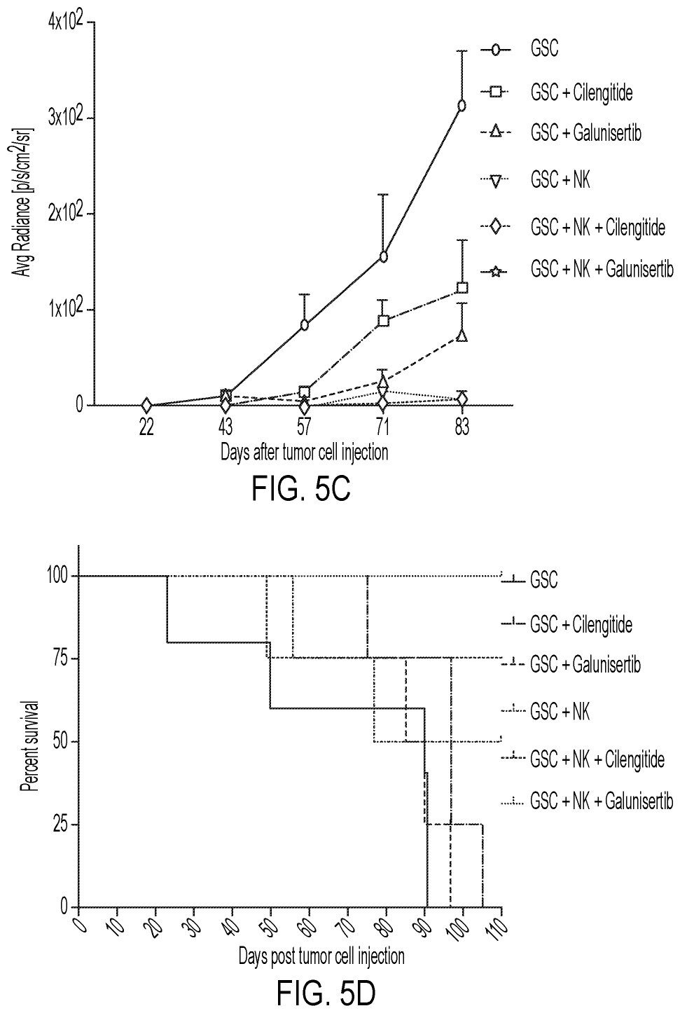

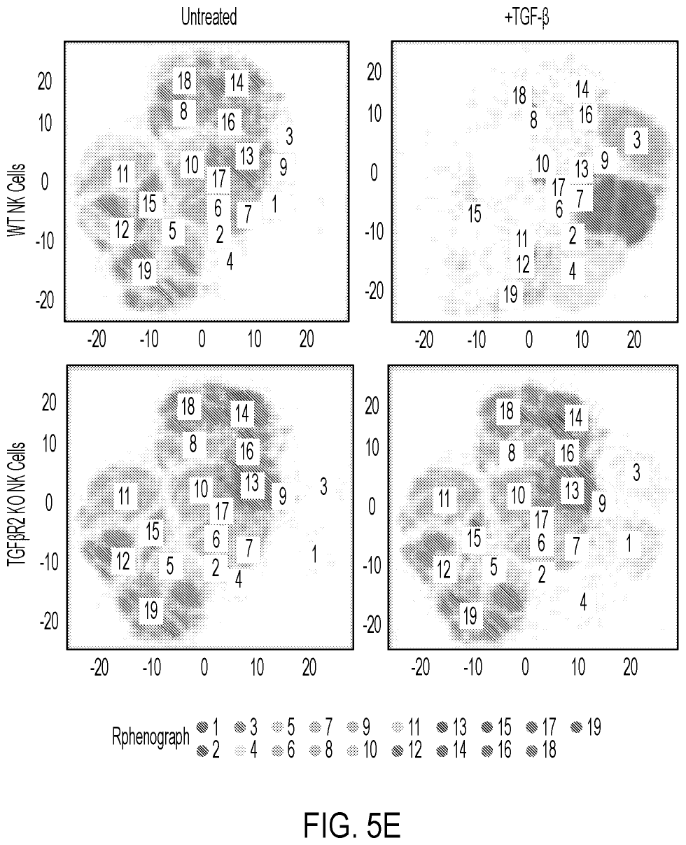

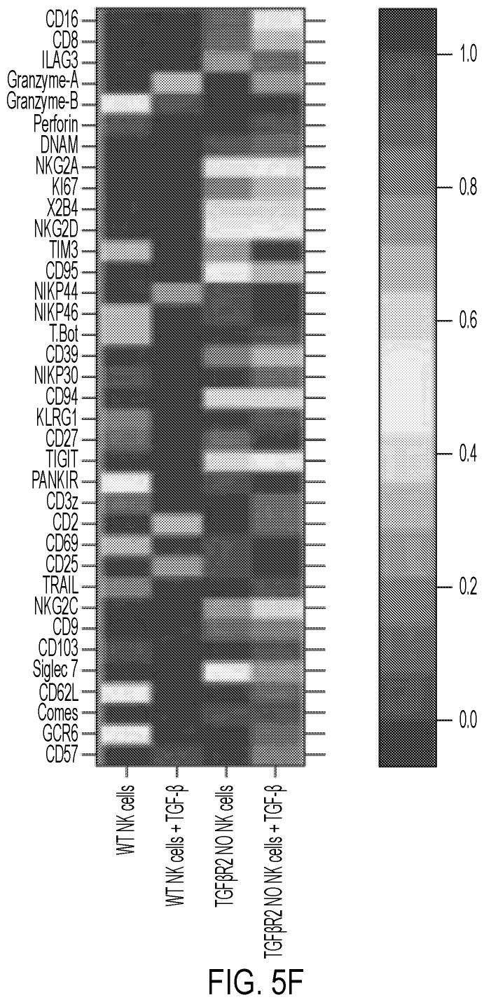

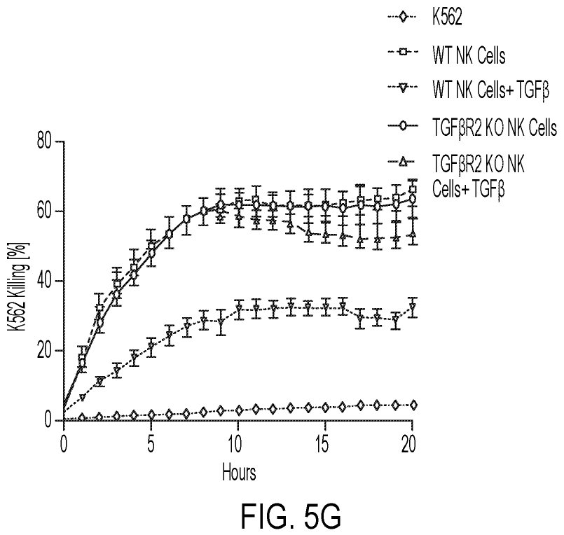

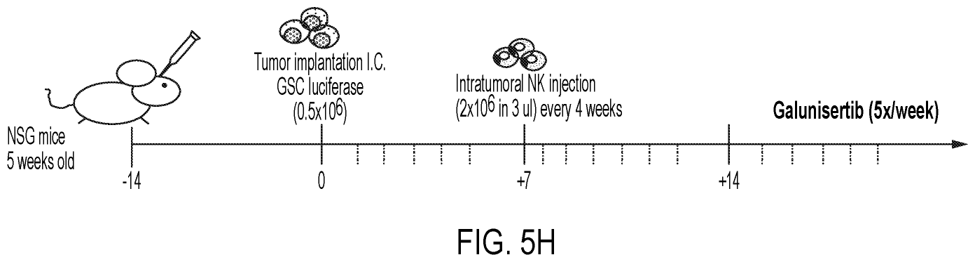

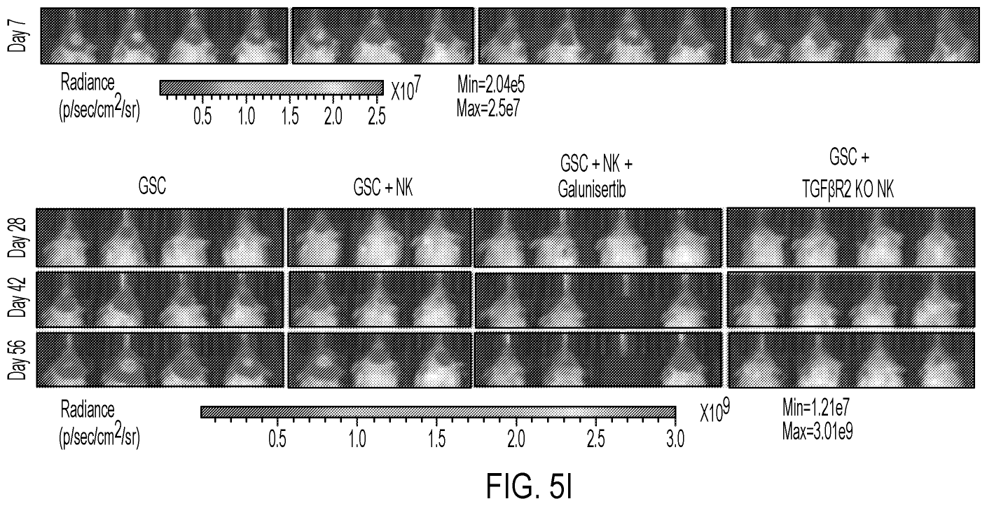

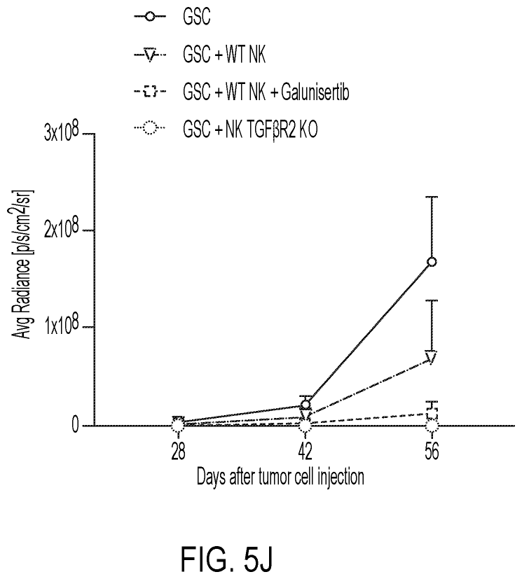

FIG. 5G shows the cytotoxicity of TGF-beta R2 knockout (KO) NK cells against GSC targets. TGF-beta R2 KO or non-engineered NK cells (NT) were cultured with 10 nM recombinant TGF-beta and their cytotoxicity was tested against K562 targets. TGF-beta R2 KO NK cells cultured in the presence or absence of recombinant TGF-beta killed K562 targets equally well, as shown by Incucyte® live imaging. Apoptotic cells are measured by caspase 3/7 green signal. On the other hand, NT NK cells cultured with TGF-beta (purple, line second from the bottom) had inferior cytotoxicity against K562 targets compared to cells cultured in the absence of TGF-beta (black, line at the top). The line at the bottom represents K562 cells alone.

Blocking of TGF-beta signaling in NK cells using galunisertib or blocking of the interaction between integrins on the surface of NK cells and TGF-beta-LAP on GBM cells using cilengitide enhances NK-mediated GBM killing in vivo. FIG. 5A-5B demonstrates bioluminescence imaging (FIGS. 5A-5C) and survival (FIG. 5D) in a PDX model of GBM. FIG. 5I shows that blocking of TGB-beta signaling in NK cells by TGF-beta R2 KO enhances NK-mediated GBM killing in vivo in a PDX model of GBM.

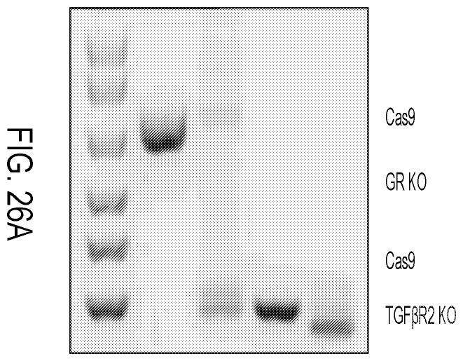

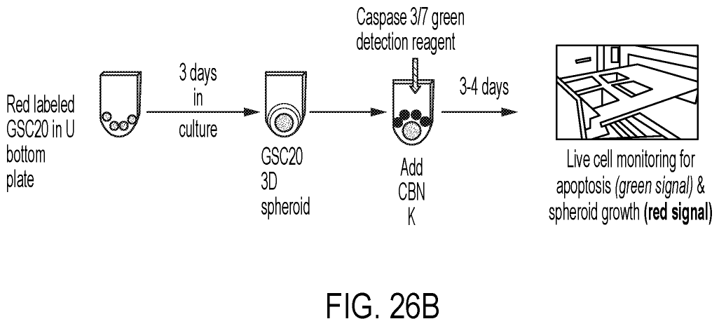

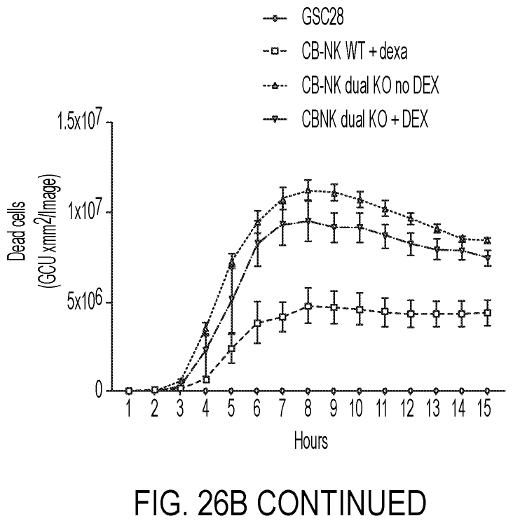

Another limitation of cell therapy in GBM is the use of corticosteroids administered to decrease edema and counter symptoms and/or adverse events. Corticosteroids are lymphocytotoxic and significantly limit the efficacy of immune cell-based therapies. Thus, in order to protect NK cells from both TGF-β-mediated and corticosteroid-induced immune suppression, the inventors have developed a novel multiplex Cas9 gene-editing approach that allows simultaneous silencing of multiple genes in primary NK cells using RNA-guided endonucleases CRISPR (clustered regularly interspaced short palindromic repeats) and CRISPR-associated (Cas) 9 gene editing (FIG. 26A). Dual silencing of TGF-β receptor 2, by CRISPR knockout (KO) of exon 5 of the TGF-βR2 gene, and of the glucocorticoid receptor (GR), by targeting exon 2 of the NR3C1 gene, results in significantly enhanced CB-NK cell cytotoxicity against GSCs, even in the presence of high doses of corticosteroids (FIG. 26B).

### Example 2

Glioblastoma Multiforme Pathobiology

Glioblastoma multiforme (GBM) or grade IV astrocytoma, is the most common and aggressive type of primary brain tumor in adults. Despite current treatment with resection, radiotherapy and temozolamide, the outcome is poor with a reported median survival of 14.6 months and a 2-year survival of 26.5% as the tumor invariably relapses^{1,2}. This dismal outcome has stimulated keen interest in immunotherapy as a means to circumvent one or more of the factors that have limited the impact of available treatments: (i) rapid growth rate of these aggressive tumors; (ii) their molecular heterogeneity and propensity to invade critical brain structures, and (iii) the tumor regenerative power of a small subset of glioblastoma stem cells (GSCs)^{3,4}.

Emerging results from preclinical studies support the concept that GBM tumors and their associated stem cells may be susceptible to immune attack by natural killer (NK) cells^{5,6,7,8,9}. These innate lymphocytes have a broad role in protecting against tumor initiation and metastasis in many types of cancer, and they have distinct advantages over T cells as candidates for therapeutic manipulation^{10,11}. However, the vast majority of tumor cells that have been studied to date possess defenses, allowing them to evade NK cellmediated cytotoxicity. These include disruption of receptor-ligand interactions between NK and tumor cells and the release of immunosuppressive cytokines into the microenvironment, such as TGF-β^{12,13,14,15}. Even if one could shield NK cells from the evasive tactics of GBM tumors, it may not be possible to eradicate a sufficient number of self-renewing GSCs to sustain complete responses. Indeed, very little is known about the susceptibility of GSCs to NK cell surveillance in vivo. Thus, to determine if GSCs can be targeted by NK cells in vivo, a preclinical study was designed and used single cell analysis of primary GBM tissue from patients undergoing surgery to determine the extent to which NK cells infiltrate sites of active tumor and the potency with which they eliminate patient-derived GSCs.

Embodiments encompassed herein show that NK cells comprise one of the most abundant lymphoid subsets infiltrating GBM tumor specimens but possess an altered NK cell phenotype that correlates with reduced cytolytic function, indicating that GBM tumors generate a suppressive microenvironment to escape NK cell antitumor activity. GSCs proved highly susceptible to NK-mediated killing in vitro, but evaded NK cell recognition via a mechanism requiring direct αν integrin-mediated cell-cell contact, leading to the release and activation of TGF-β by the GCSs. In a patient-derived xenograft (PDX) mouse model of glioblastoma, GSC-induced NK dysfunction was completely prevented by integrin or TGF-β blockade or by CRISPR gene editing of the TGF-β receptor 2 (TGFβR2) on NK cells, resulting in effective control of the tumor. Taken together, these data suggest that inhibition of the αν integrin-TGF-β axis could overcome a major obstacle to effective NK cell immunotherapy for GBM.

### Example 3

Gscs are Susceptible to Nk Cell-Mediated Killing

The GSCs can be distinguished from their mature tumor progeny at the transcriptional, epigenetic and metabolic levels^{16,17}, raising the question of whether these cells can be recognized and killed by NK cells. The question arises as to whether patient-derived GSCs, defined as being capable of self-renewal, pluripotent differentiation, and tumorigenicity when implanted into an animal host, are susceptible to NK cell cytotoxic activity as compared with healthy human astrocytes. GSCs were derived from patients with various glioblastoma subtypes including mesenchymal (GSC20, GSC267), classical (GSC231, GSC6-27), and proneural (GSC17, GSC8-11, GSC262) while also showing heterogeneity in the O(6)-Methylguanine-DNA methyltransferase (MGMT) methylation status (methylated: GSC231, GSC8-11, GSC267; indeterminate: GSC6-26, GSC17, GSC262). K562 targets were used as positive control because of their marked sensitivity to NK cell mediated killing due to lack of expression of HLA class I¹⁸. Across all effector:target (E:T) ratios, healthy donor NK cells killed GSCs (n=6) and K562 cells with equal efficiency and much more readily than healthy human astrocytes (n=6), which displayed a relative resistance to NK cell-mediated killing (FIG. 1A). Multi-parametric flow cytometry was then used to analyze the expression of NK cell activating or inhibitory receptor ligands on GSCs. GSCs (n=6) expressed normal levels of HLA-class I and HLA-E (both ligands for inhibitory NK receptors), at levels similar to those observed on healthy human astrocytes (n=3) (FIG. 1B). In contrast, the ligands for activating NK receptors, such as CD155 (ligand for DNAM1 and TIGIT), MICA/B and ULBP1/2/3 (ligands for NKG2D) and B7-H6 (ligand for NKp30) were upregulated on GSCs but not on healthy human astrocytes (FIG. 1B).

To assess the contributions of these activating and inhibitory receptors to the NK cell-dependent cytotoxicity against GSCs, receptor-specific blocking antibodies were used to disrupt specific receptor-ligand interactions. The blockade of NKG2D, DNAM1 and NKp30 but not HLA class I, significantly decreased NK cell-mediated GSC killing (n=4) (FIG. 1C). Cumulatively, these findings suggest that GSCs possess the ligands needed to stimulate NK cell activation leading to GSC elimination. Indeed, the effects observed were entirely consistent with an extant model of tumor cell attack by NK cells, whereby inhibitory signals transmitted by KIR-HLA class I interactions are overcome when a threshold level of activating signals are reached, inducing recognition of ‘stressed’ cells.^{19,20,21,22}

### Example 4

Nk Cells Infiltrate Gbm Tumors but Display an Altered Phenotype and Function

Preclinical findings in glioma-bearing mice indicate that NK cells can cross the blood-brain barrier to infiltrate the brain²³. However, the limited clinical studies available suggest only minimal NK cell infiltration into GBM tissue²⁴. As such, it was investigated whether NK cells are capable of infiltrating into GBMs and their abundance by analyzing ex vivo resected glioma tumor specimen collected from 21 of 46 patients with primary or recurrent GBM, and 2 of 5 patients with low-grade gliomas. Each gram of GBM contained a median of 166,666 NK cells (range 9,520-600,000; n=21) whereas there were only 500-833 NK cells/g in low-grade gliomas (n=2). These findings indicate that NK cells can traffic into the GBM microenvironment in numbers that appear to be much larger in high-grade gliomas.

Cytometry by time-of-flight (CyToF) and a panel of 37 antibodies against inhibitory and activating receptors, as well as differentiation, homing and activation markers (Table 1) were used to gain insights into the phenotype of the GBM tumor-infiltrating NK cells (TiNKs). Uniform manifold approximation and projection (UMAP), a dimensionality reduction method, was run on a dataset from paired peripheral blood NK cells (PB-NK) and TiNKs from patients with GBM and peripheral blood from healthy controls. Heatmap was used to compare protein expression between the groups. This transcriptomic profiles of TiNKs from 10 additional glioma patients and PBMCs from healthy donors using a Drop-Seq-based scRNA-seq technology (10x Genomics STAR Methods) from a soon to be publicly available dataset of CD45+ glioma infiltrating immune cells [Zamler et al. Immune landscape of genetically engineered murine models of glioma relative to human glioma by single-cell sequencing Manuscript in Submission. (2020)]. Over 1746 NK cells from each GBM patient sample and over 530 cells from each healthy PBMC donor were used. The NK signature used to define the NK population included the markers KLRD1, NKG7 and NKTR. There was significant downregulation of the genes that encoded NK cell activation markers such as NCR3 [NKp30], GZMA [granzyme A], GZMK [granzyme K], SELL [CD62L], FCGR3A [CD16] and CD247 [CD3Z] on TiNKs from GBM patients compared with healthy donor PBMCs (HC-NK) (FIG. 1F). Genes that encoded for NK cell inhibitory receptors such as KLRD1 [CD94], KIR2DL1 and KIR2DL4 were upregulated on the TiNKs compared to the HC-NKs (FIG. 1F). Interestingly, genes associated with TGFβ pathway as JUND, SMAD4, SMAD7 and SMURF2 were also significantly upregulated on TiNKs compared with HCNK (FIG. 1F).

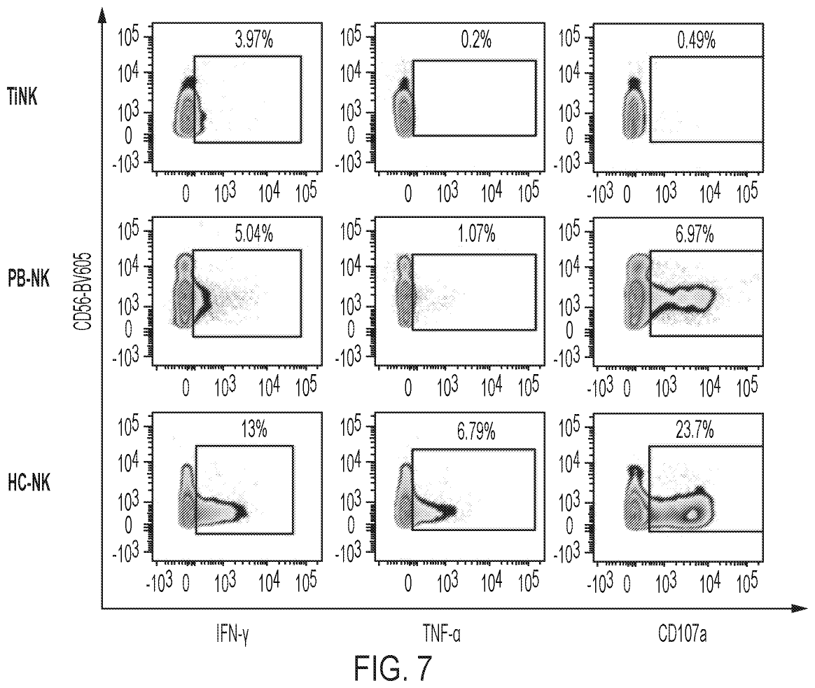

The impact of the phenotypic findings on NK cell function was tested by isolating NK cells from the GBM tumor or PB-NK cells and testing their effector function against K562 targets. TiNKs exerted less cytotoxicity by ⁵¹Cr release assay, less degranulation (reduced expression of CD107a) and produced significantly lower amounts of IFN-7 and TNF-α than did PB- or HC-NK (FIGS. 2A-2B; FIG. 7). Taken together, these data indicate that NK cells can indeed migrate into GBMs and undergo immune alteration within the tumor microenvironment that results in marked impairment of their cytotoxic function, indicating their susceptibility to immune evasion tactics of the malignant tumor.

### Example 5

Tgf-β1 Mediates NK Cell Dysfunction in GBM Tumors

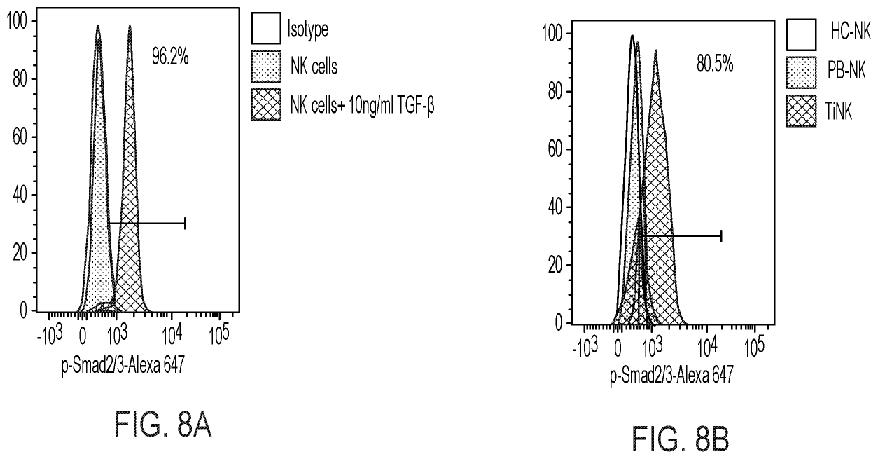

Despite the intrinsic sensitivity of GSCs to immune attack by NK cells, the findings indicate that this sensitivity is partially lost within the tumor microenvironment, where TiNKs are modulated toward an inhibitory phenotype. Although there are many different mechanisms that could account for this shift in function¹², the TiNK phenotypic and single cell transcriptomic alterations were most consistent with the effects of TGF-β1, a pleiotropic cytokine that functions as an important inhibitor of the mTOR pathway²⁵. This notion was supported by the observation of enhanced basal levels of p-Smad⅔, the canonical TGF-β signaling pathway, in TiNK cells compared to PB- or HC-NK cells (FIG. 2C; FIG. 8).

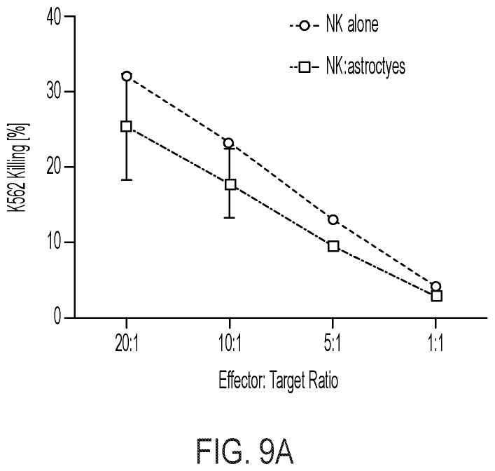

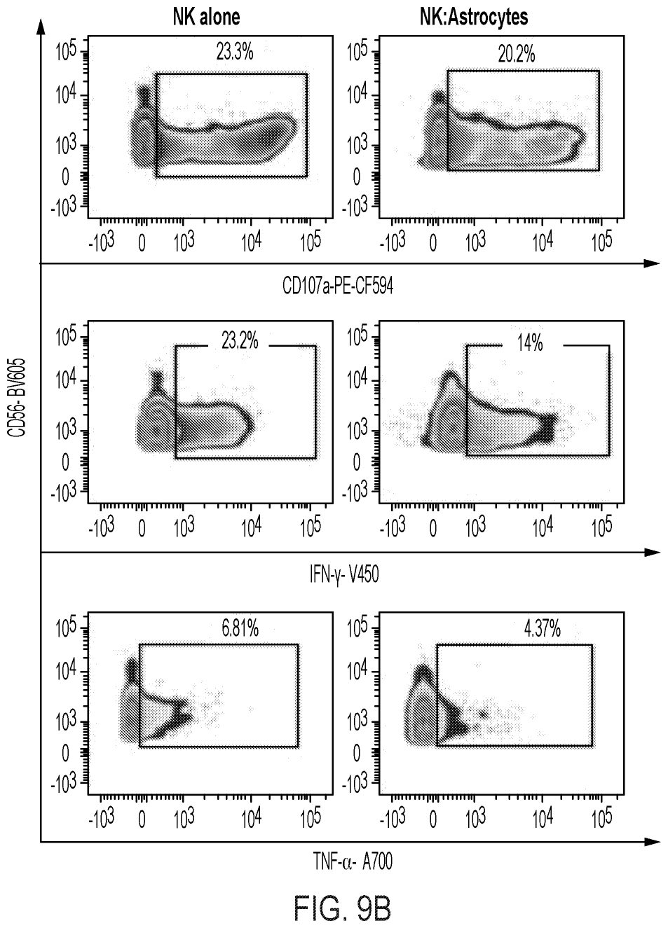

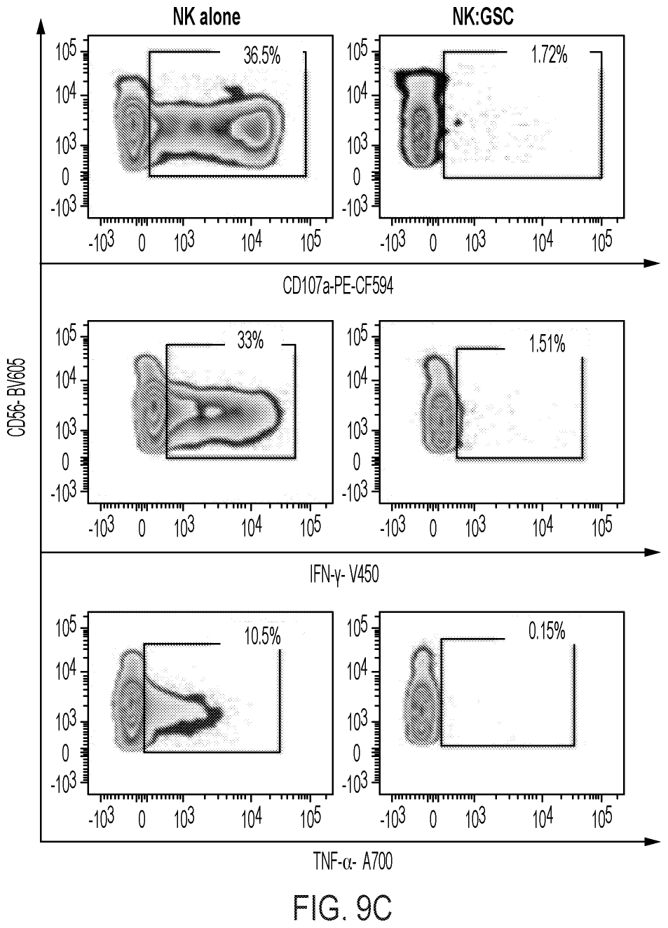

Given the rarity of the GSCs and their exquisite sensitivity to NK cell cytotoxicity, it could be reasoned that they may have evolved their own mechanisms of immune evasion in addition to the evasive tactics provided by the known immune regulatory cells in the microenvironment¹². To pursue this hypothesis, it was tested whether GSCs can suppress the function of healthy allogeneic NK cells in vitro. While incubation with healthy human astrocytes (control) had no effect on NK cell function (n=3) (FIGS. 9A-9B), co-culture with patient-derived GSCs significantly impaired the ability of allogeneic NK cells to perform natural cytotoxicity and to produce IFN-7 and TNF-α in response to K562 targets (n=10; n=15 respectively) (FIGS. 2D-2E; FIG. 9C). Next, it was tested whether TGF-β1 plays a role in GSC-induced NK cells dysfunction by co-culturing NK cells from healthy control donors with patient-derived GSCs in the presence or absence of TGF-β neutralizing antibodies and assessing their cytotoxicity against K562 targets. While the antibodies did not affect the normal function of healthy NK cells when cultured alone (FIG. 10A), the blockade of TGF-β1 prevented GSCs from disabling NK cell cytotoxicity (FIGS. 10B-10D). Thus, TGF-β1 production by GSCs contributes significantly to NK cell dysfunction in the GBM microenvironment.

### Example 6

Disruption of TGF-β1 Signaling Prevents but does not Reverse GSC-Induced NK Cell Dysfunction

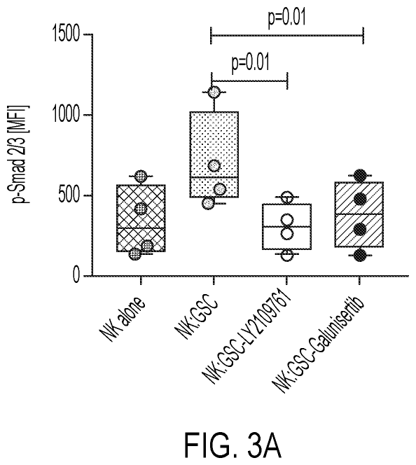

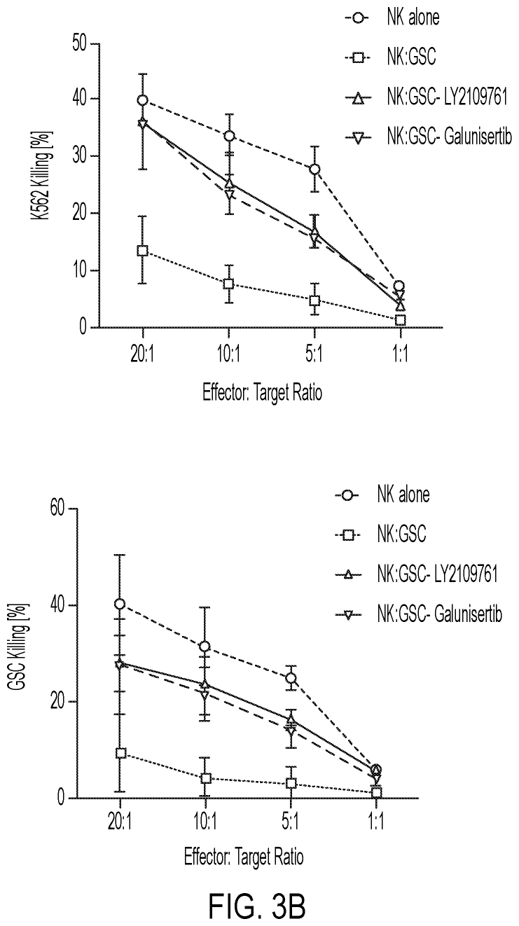

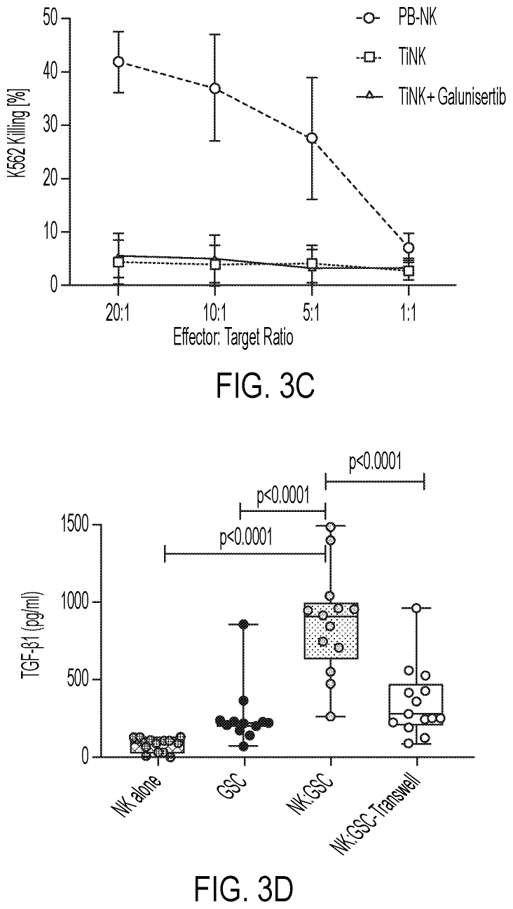

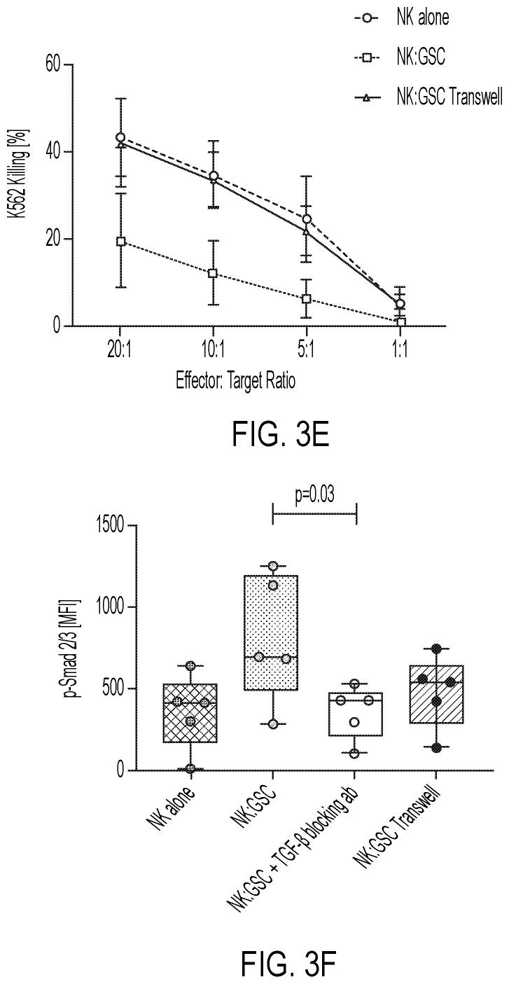

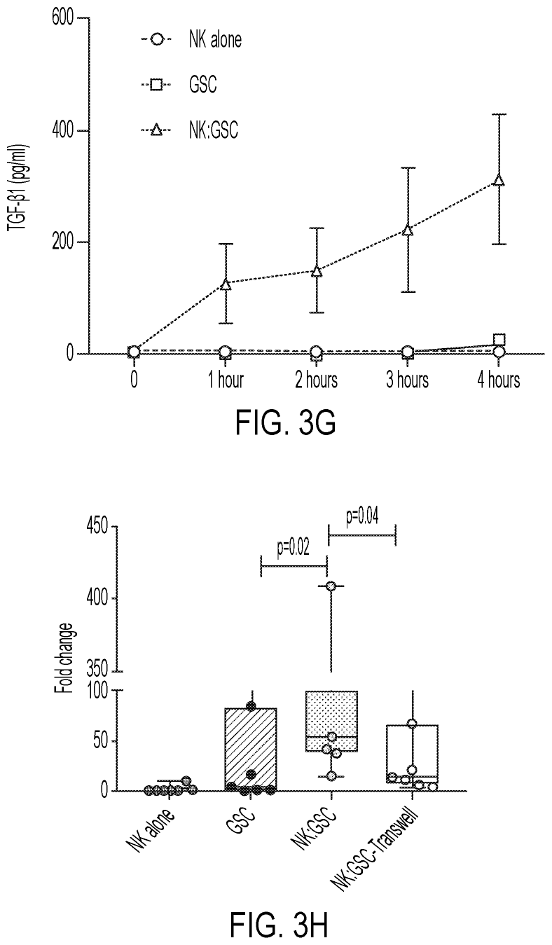

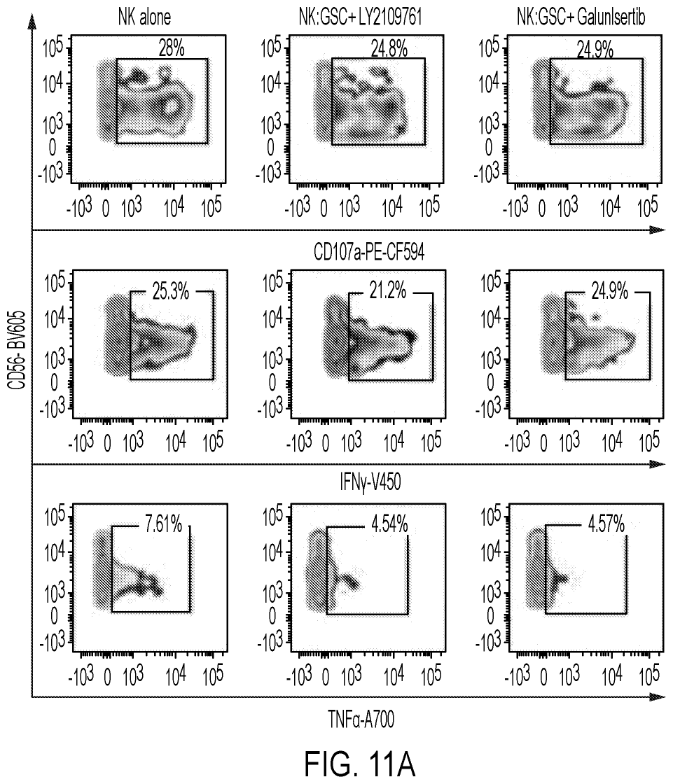

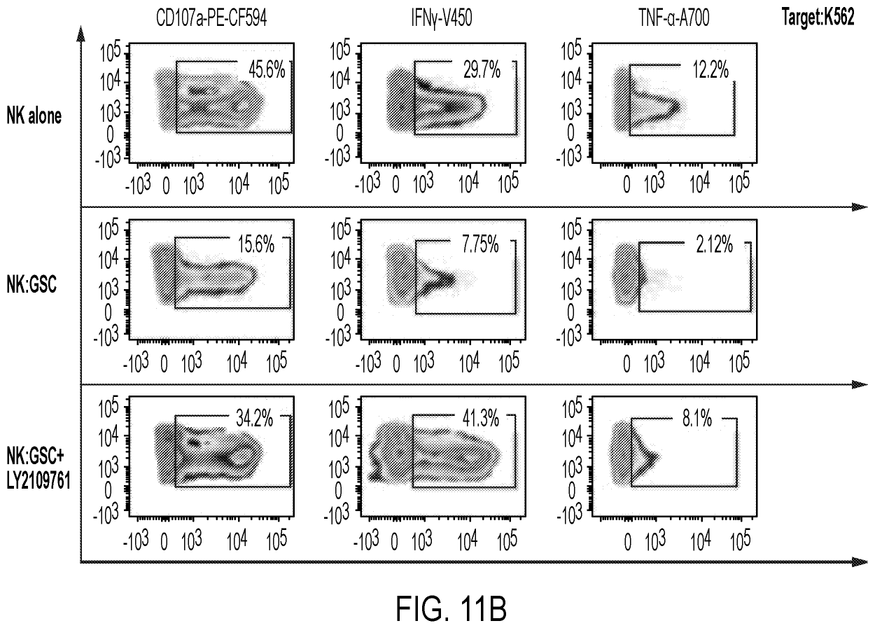

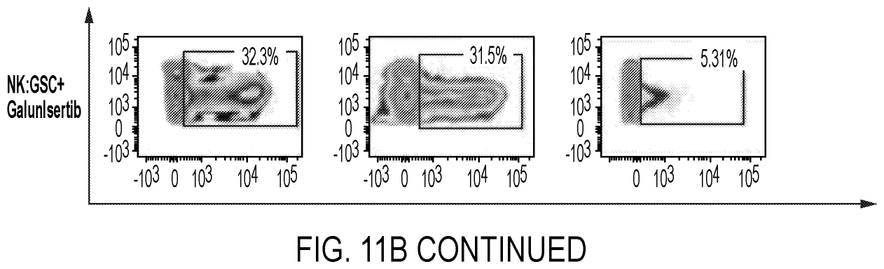

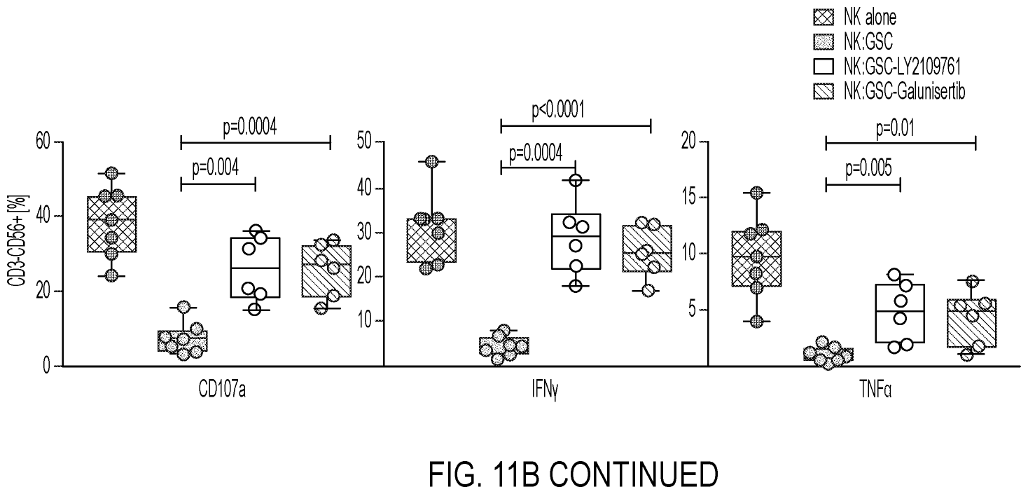

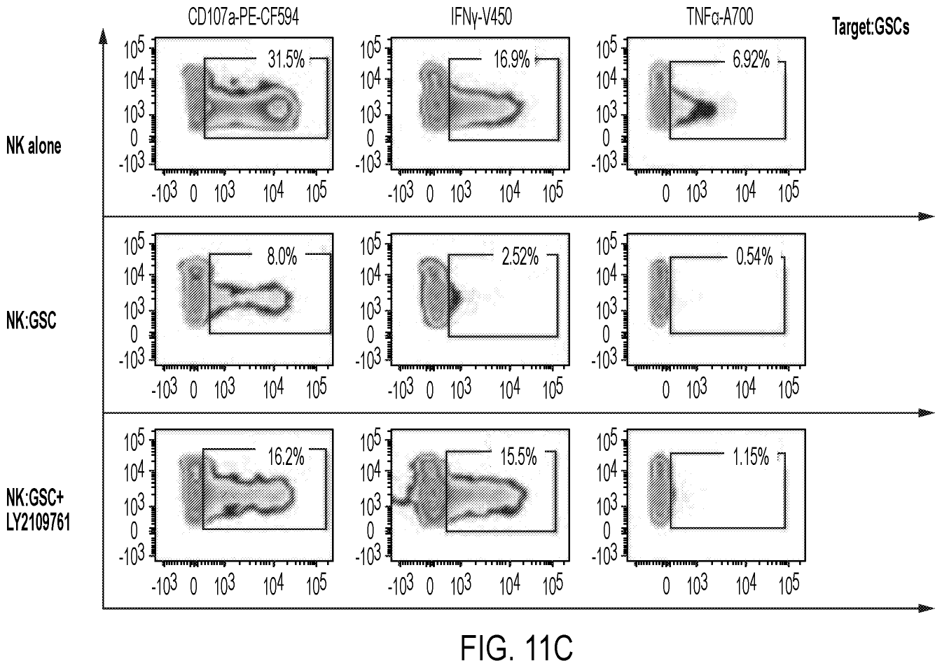

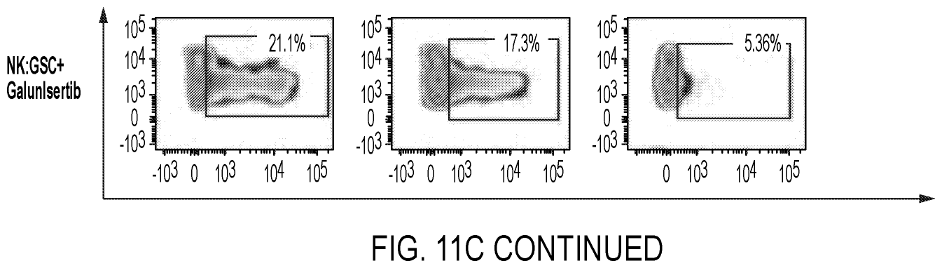

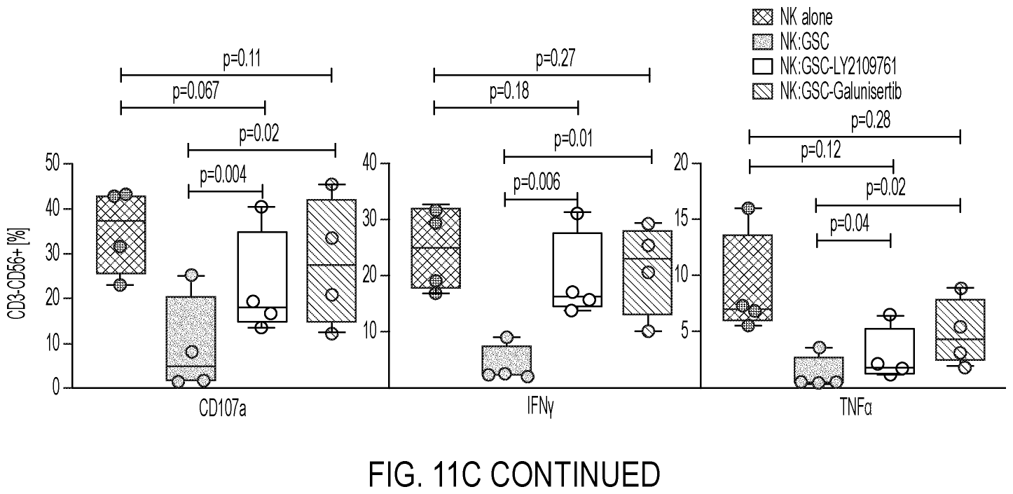

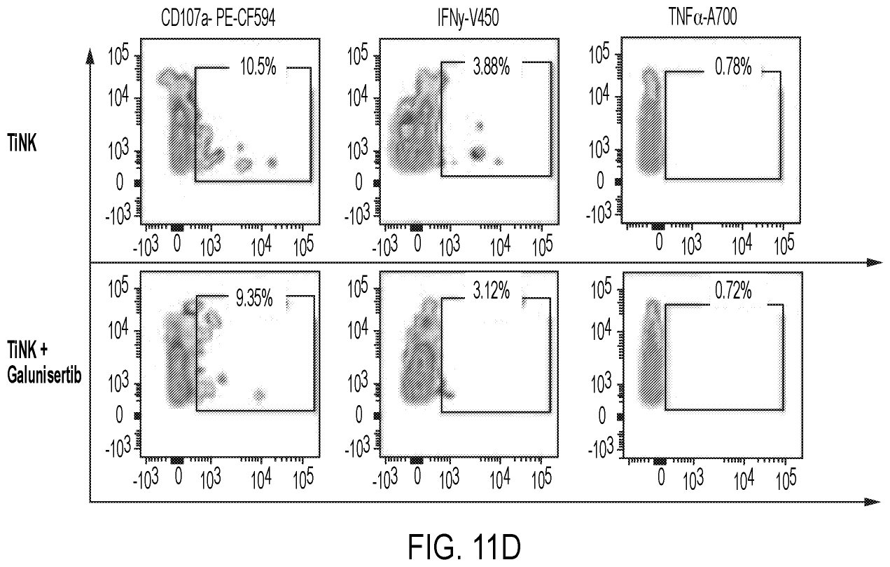

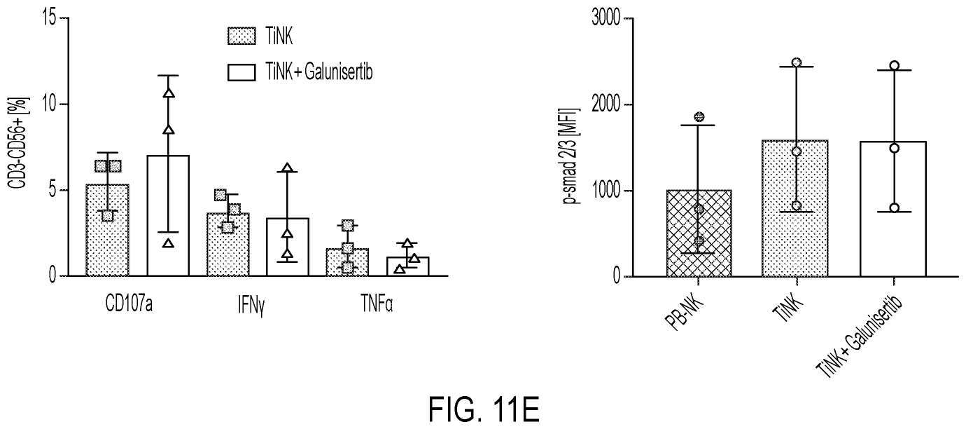

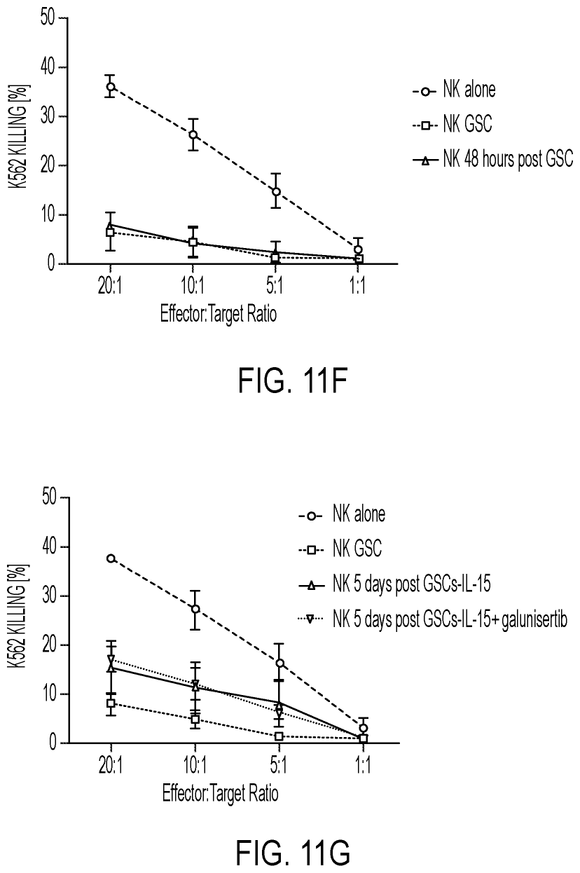

If GSCs induce NK cell dysfunction through activation and release of TGF-β1, it may be possible to avoid this evasive tactic by inhibiting the TGF-β signaling pathway. Thus, it was first tested whether galunisertib (LY2157299), a TGF-β receptor I kinase inhibitor that has been used safely in GBM patients²⁶, and LY2109761, a dual inhibitor of TGF-β receptors I and II,^{27,28} can prevent or reverse GSC-induced NK cell dysfunction. Although neither inhibitor affected NK cell function (FIG. 11A), each prevented GSCs from activating the TGF-β1 Smad⅔ signaling pathway in NK cells (FIG. 3A) and inducing dysfunction, thus preserving the natural cytotoxicity of NK cells against K562 or GSC targets (FIG. 3B; FIGS. 11B-11C). Interestingly, blockade of the TGF-β receptor kinase by galunisertib or ex vivo culture of TiNKs with activating cytokines such as IL-15 failed to inactivate the TGF-β1 Smad⅔ signaling pathway and restore NK cell dysfunction (FIG. 3C; FIGS. 11D-11E). Similarly, these maneuvers did not reverse the dysfunction of HC-NK cells induced by GSCs (FIGS. 11F-11G) indicating that once NK cells are rendered dysfunctional in the suppressive microenvironment of GBM tumors, stimulation with IL-15 or inhibition of TGF-β 1 activity is unlikely to restore their function.

### Example 7

Gscs Induce Nk Cell Dysfunction Through Cell-Cell Contact Dependent TGFβ Release

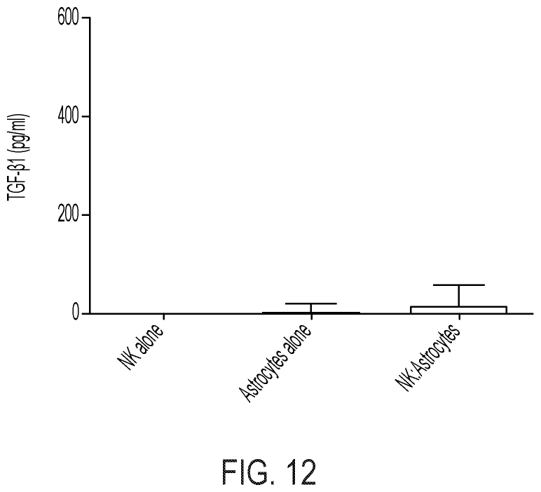

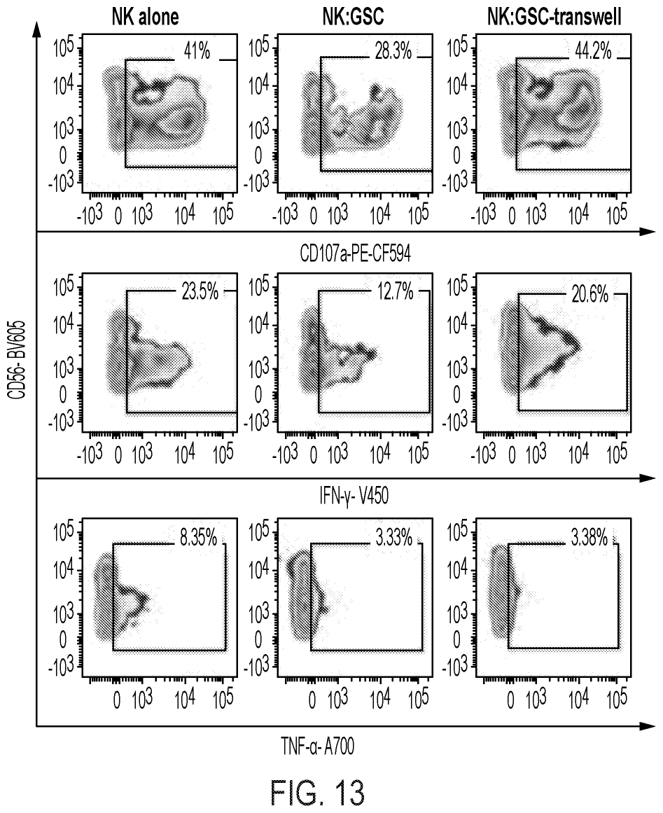

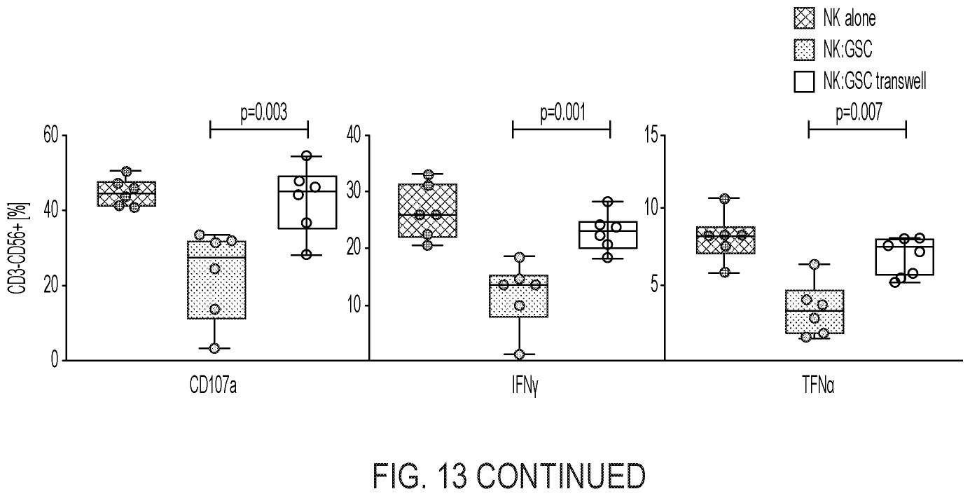

A question arose as to whether TGF-β1 secretion by GSCs is an endogenous process, as observed with macrophages and myeloid-derived suppressor cells (MDSCs)^{29,30}, or requires active cell-cell interaction with NK cells. To address this question, transwell experiments were performed in which healthy donor-derived NK cells and GSCs were either in direct contact with each other or separated by a 0.4 m pore-sized permeable membrane that allowed the diffusion of soluble molecules, but not cells. Levels of soluble TGF-β1 were measured 48 hours after the cultures were initiated. Direct contact of GSCs with NK cells resulted in significantly higher levels of TGF-β1 compared with those attained when GSCs were separated from NK cells by a transwell (mean 836.9 pg/ml±333.1 S.D. vs 349 pg/ml±272.2 S.D.) or when GSCs were cultured alone (252±190.4 pg/ml; p<0.0001) (FIG. 3D), indicating that activation and secretion of TGF-β by GSCs is a dynamic process requiring direct cell-cell contact between the NK cells and GSCs. Importantly, healthy human astrocytes cultured either alone or with NK cells did not produce substantial amounts of TGF-β1 (FIG. 12). Consistent with these results, it was found that GSC-mediated NK cell dysfunction also required direct cell-cell contact. Indeed abrogation of direct cell-cell contact between NK cells and GSCs by a transwell membrane prevented the induction of NK cell dysfunction, and activation of the TGF-β1 Smad⅔ pathway, similar to results with TGF-β1 blocking antibodies (FIGS. 3E-3F; FIG. 13).

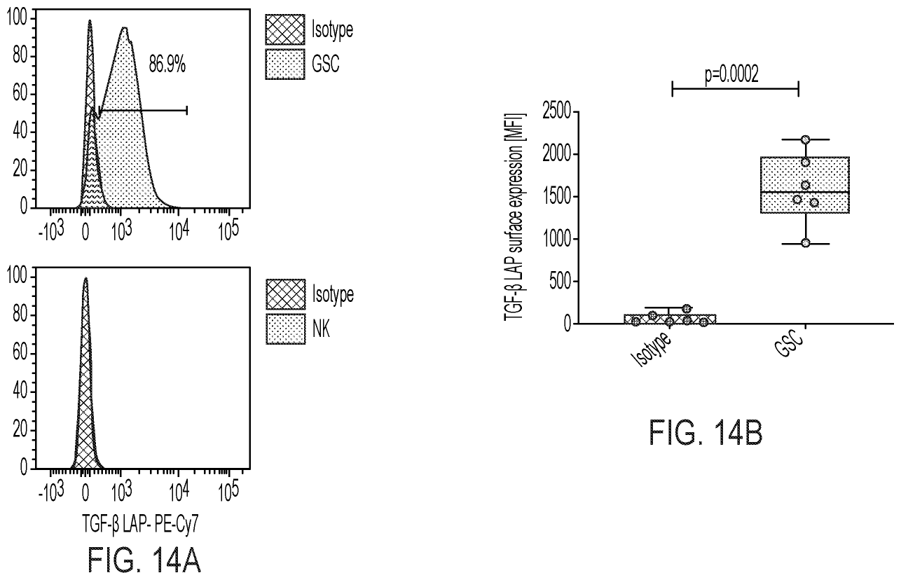

TGF-β1 is a tripartite complex and its inactive latent form is complexed with two other polypeptides: latent TGF-β binding protein (LTBP) and latency-associated peptide (LAP). Activation of the mature TGF-β1 requires its dissociation from the engulfing LAP. Because TGF-β1-LAP is expressed on the surface of GSCs at high levels (FIGS. 14A-14B), experiments were performed to determine if the increase in soluble TGF-β levels in the supernatant after GSC-NK cell contact was driven by release of the cytokine from the engulfing LAP or by increased transcription of the TGF-β1 gene, or both. To distinguish between these two alternatives, it was investigated if contact with NK cells can induce a rapid release of TGF-β from LAP by measuring the kinetics of TGF-β1 production in the supernatant after GSC-NK cell coculture. The results indicate a rapid increase in soluble TGF-β 1 levels as early as 1 hour after co-culture in conditions where NK cells and GSCs were in direct contact compared with co-cultures in which NK cells and GSCs were cultured alone (FIG. 3G). When the fold-changes in TGF-β1 mRNA were determined by quantitative PCR (qPCR) in GSCs alone or in direct contact with NK cells or separated from NK cells by a transwell membrane for 48 hours, the TGF-β1 copy numbers were significantly higher in GSCs in direct contact with NK cells (p=0.04) (FIG. 3H). Thus, the marked increase in TGF-β1 seen after NK cell interaction with GSCs appears to involve a dual mechanism of upregulated TGF-β1 transcription and release of the mature cytokine from the LAP peptide by GSCs.

### Example 8

MMP2 and MMP9 Play a Critical Role in the Release of Activated TGF-β1 from Lap

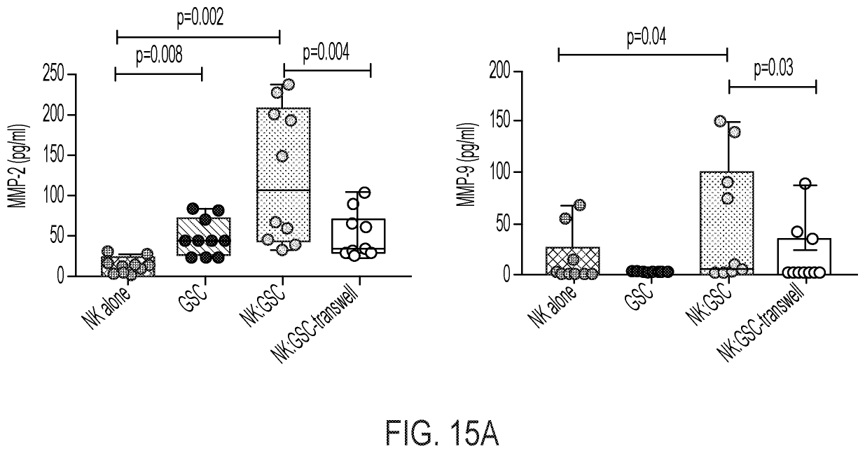

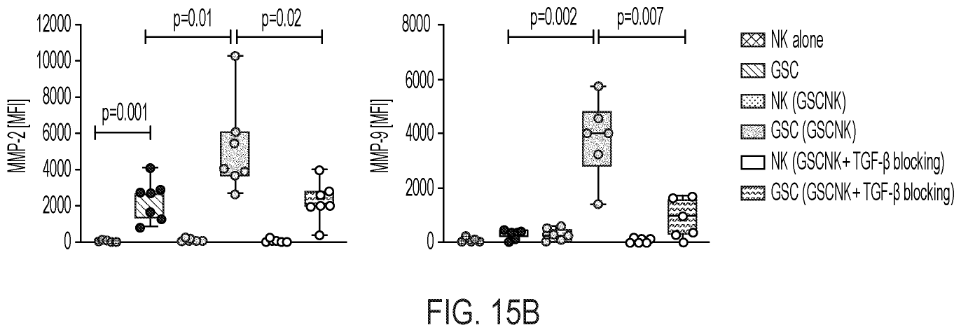

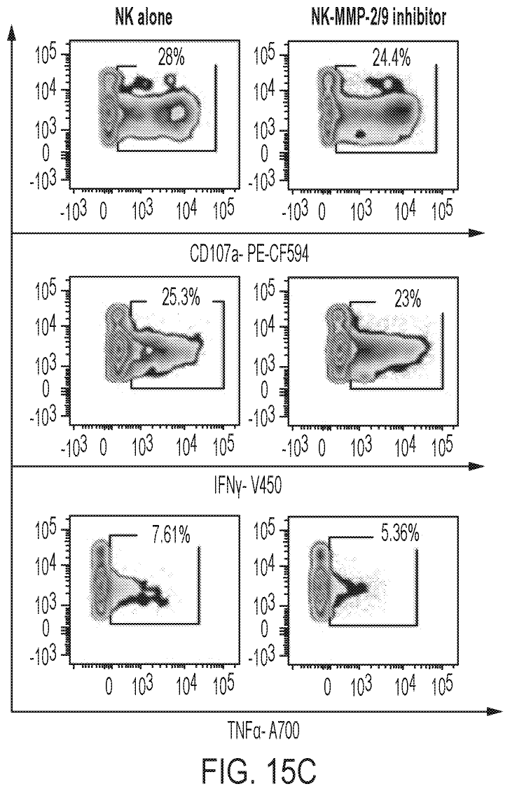

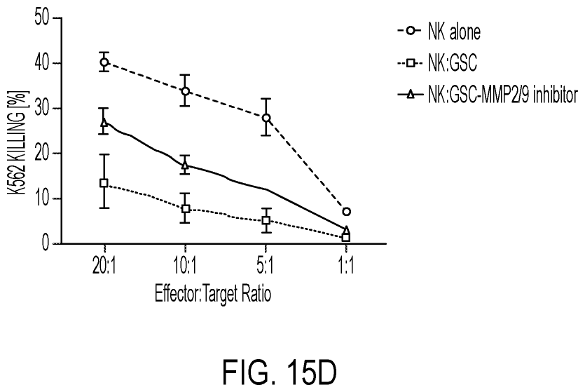

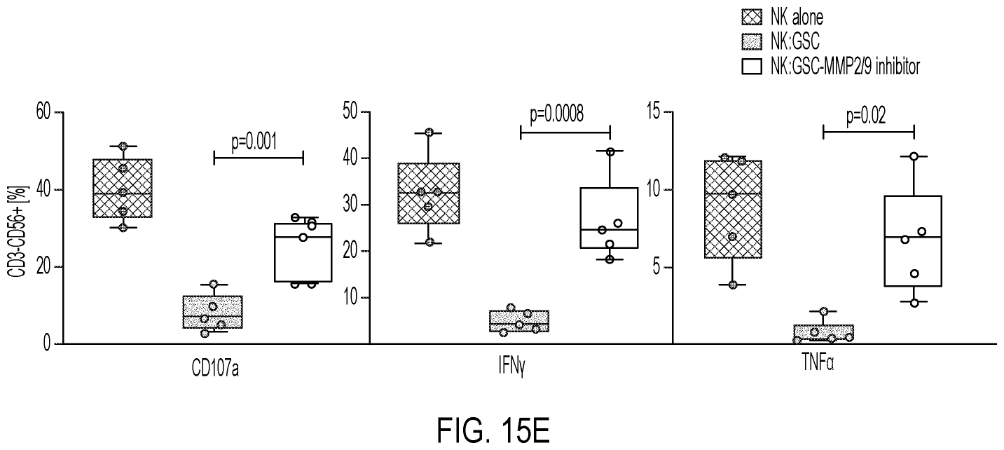

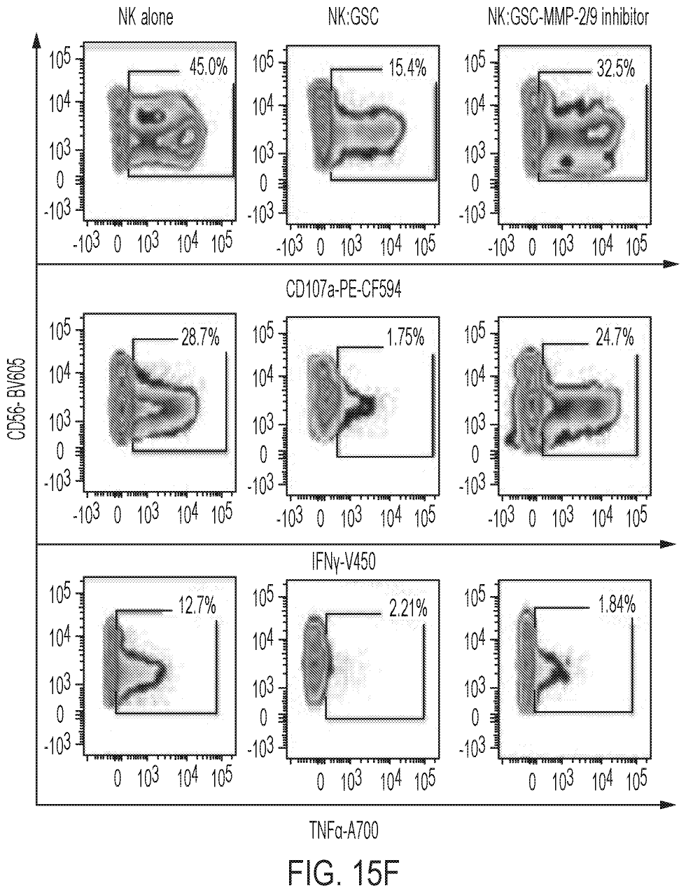

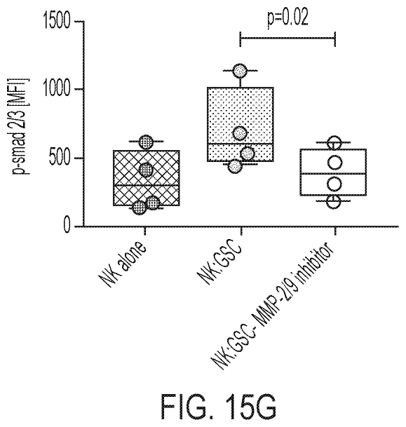

Both matrix metalloproteinases (MMPs) 2 and 9 mediate the release of TGF-β1 from LAP^{31,32}. Because both enzymes are expressed by malignant gliomas³³, it was investigated whether they might also be involved in the release of TGF-β 1 from LAP and consequently in the induction of NK cell dysfunction by GSCs. First, it was confirmed that GSCs are a major source of MMP2 and MMP9 (FIGS. 15A-15B), and then their contribution to the release of TGF-β1 and GSC-induced NK cell dysfunction was determined by culturing healthy NK cells with or without GSCs and in the presence or absence of an MMP 2/9 inhibitor for 48 hours. MMPs were present at higher levels when GSCs were in direct contact with NK cells, suggesting that TGF-β1 drives their release, as confirmed by experiments using TGF-β blocking antibodies (FIGS. 15A-15B). The addition of an MMP 2/9 inhibitor did not affect NK cell function in cultures lacking GSCs (FIG. 15C) but partially prevented GSC-induced NK dysfunction, as measured by the ability of the NK cells to perform natural cytotoxicity and to produce IFN-γ and TNF-α in response to K562 targets (FIGS. 15D-15F). This partial restoration would be consistent with the involvement of additional pathways in the activation of TGF-β. Incubation of NK cells with the MMP 2/9 inhibitor also resulted in decreased p-Smad⅔ levels (FIG. 15G), implicating MMP 2/9 in the release of TGF-β by GSCs.

### Example 9

αν Integrins Mediate Cell Contact Dependent TGF-β1 Release by GSCS

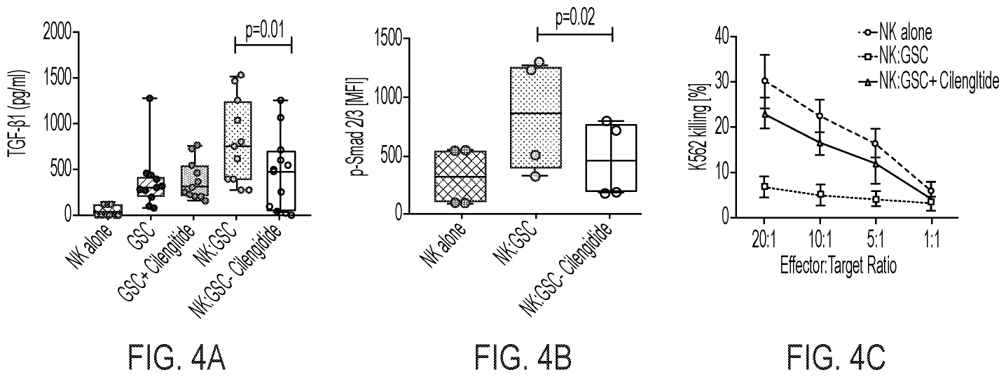

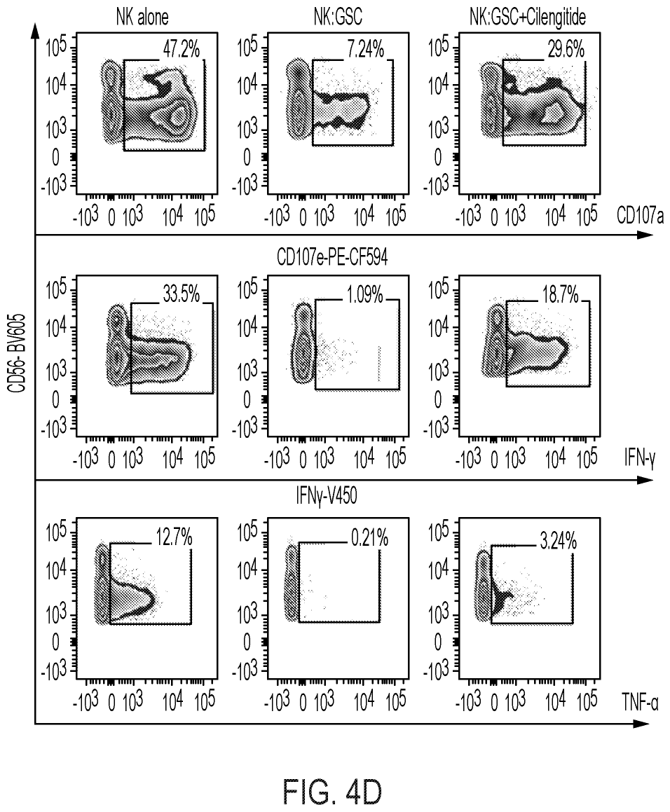

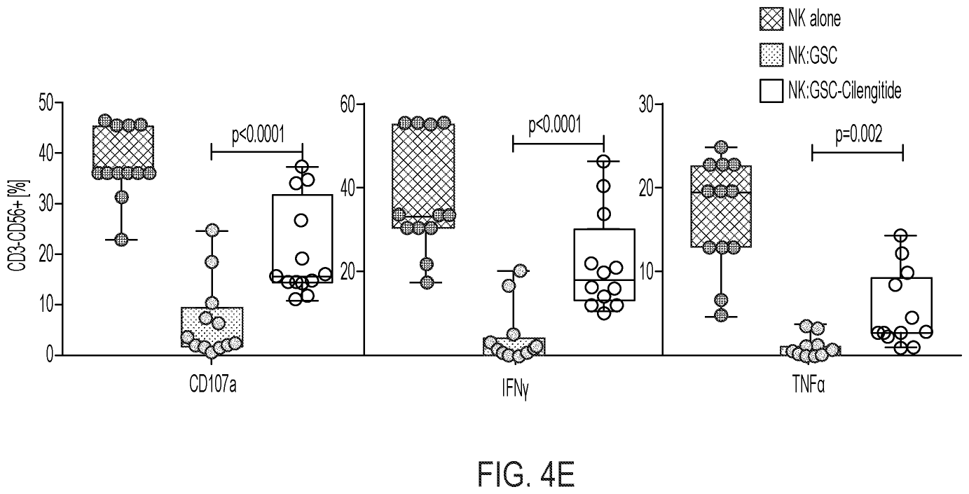

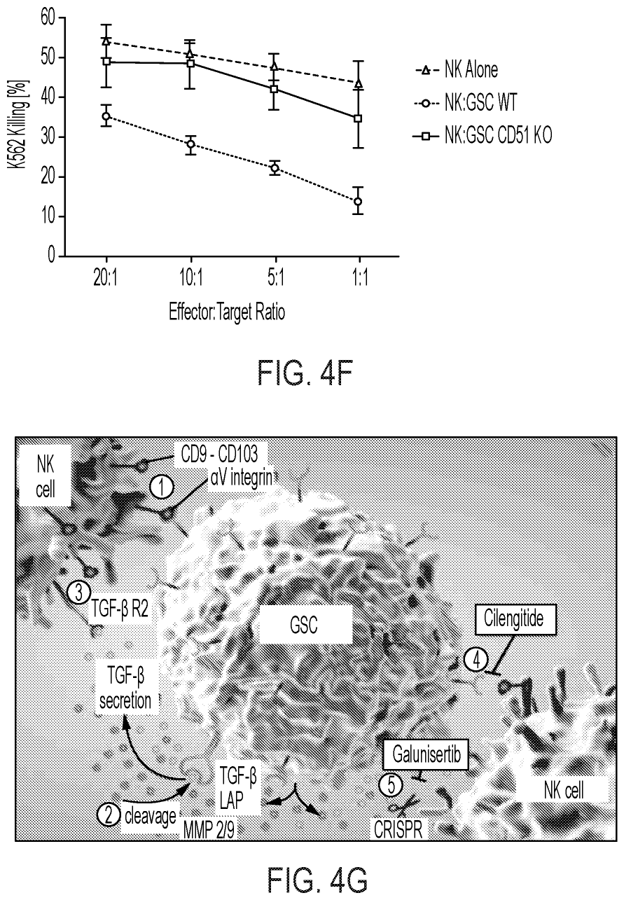

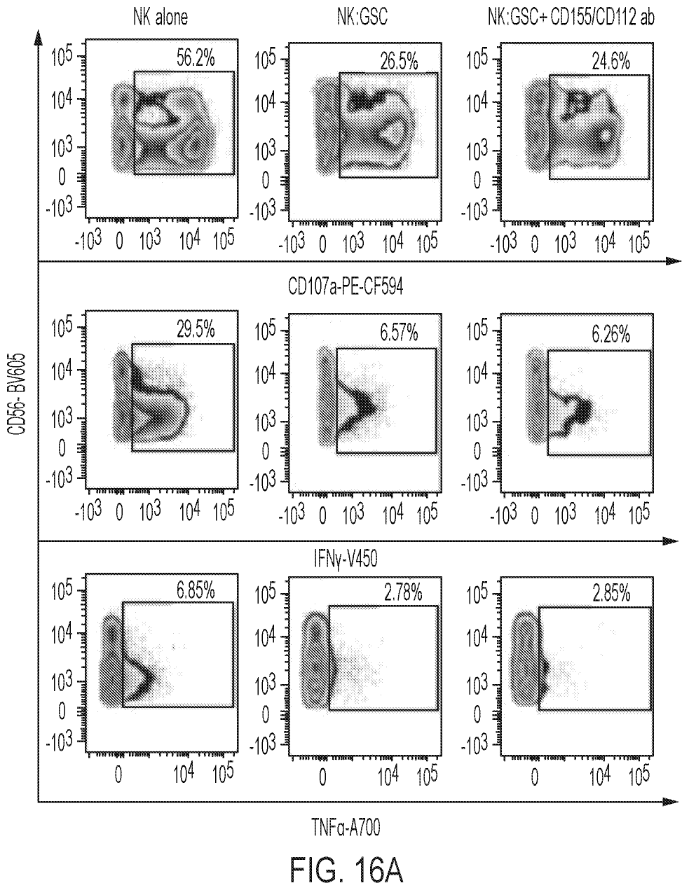

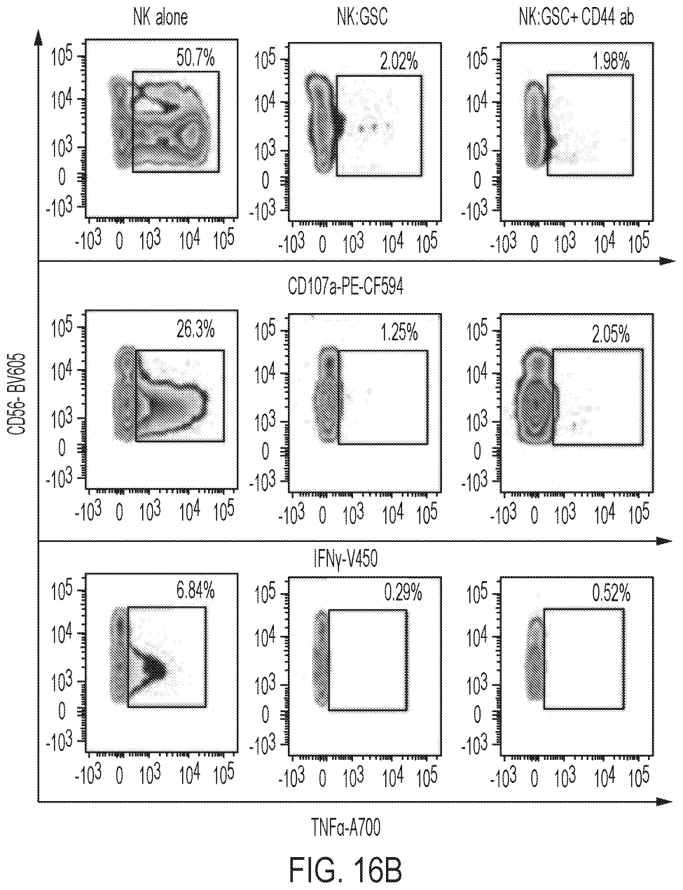

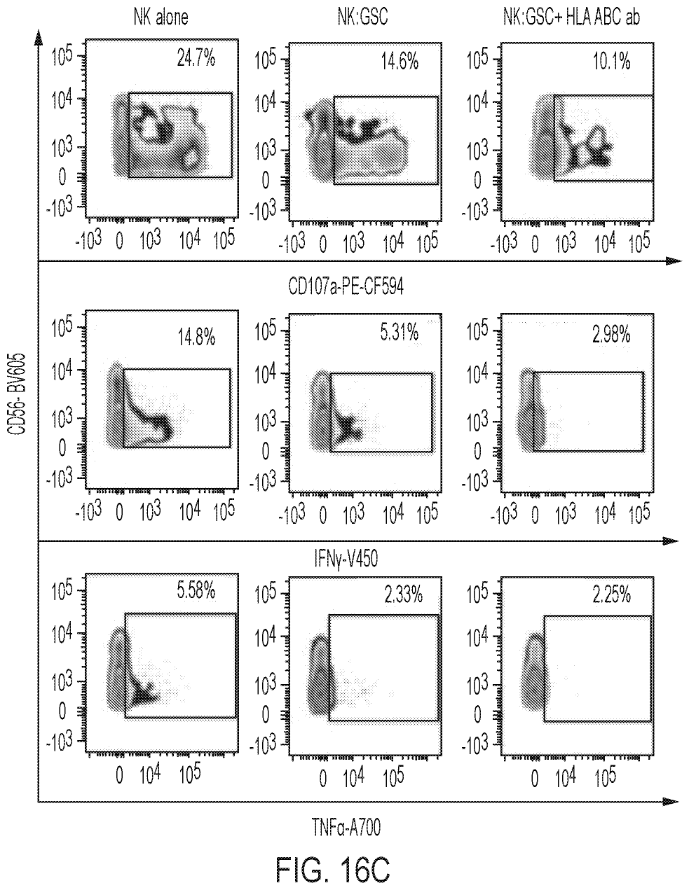

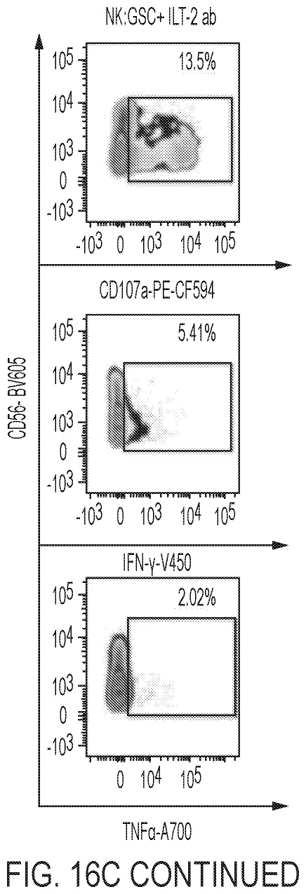

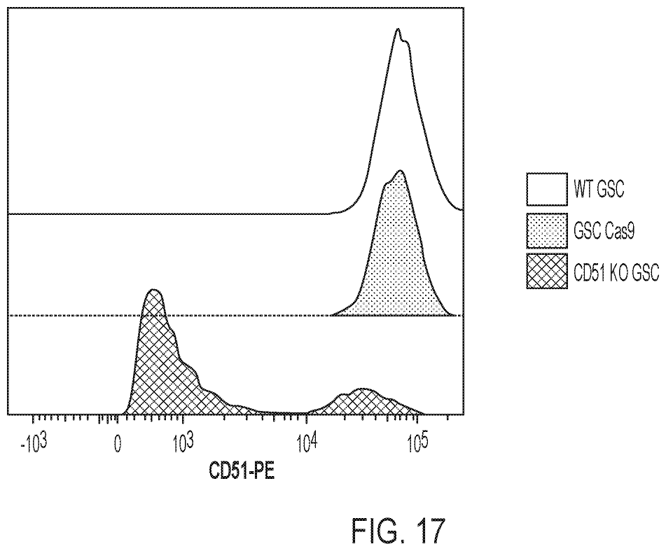

Since GSC-mediated NK cell dysfunction requires direct cell-cell contact, it was next investigated which receptor-ligand interactions could be participating in this crosstalk. Blocking the interaction of major activating and inhibitory NK cell receptors, including CD155/CD112, CD44, KIRs and ILT-2, on healthy donor NK cells and their respective ligands on GSCs failed to prevent GSC-induced NK cell dysfunction (FIGS. 16A-16C). Focus was shifted to the integrins, a family of cell surface transmembrane receptors that play a critical role not only in cell adhesion, migration and angiogenesis, but also in the activation of latent TGF-β1³⁴. The αν (CD51) integrin heterodimeric complexes ανβ3, ανβ5 and ανβ8 are highly expressed in glioblastoma, in particular on GSCs³⁵. Based on evidence that targeting αν integrins in glioblastoma can significantly decrease TGF-β production,³⁵it was tested whether cilengitide, a small molecule inhibitor that possesses a cyclic RDG peptide with high affinity for αν integrins can prevent GSC-induced NK cell dysfunction by decreasing TGF-β1 production. Treatment with cilengitide significantly decreased levels of soluble TGF-β1 (FIG. 4A) as well as p-Smad⅔ signaling in NK cells in direct contact with GSCs (FIG. 4B) and prevented GSC-induced NK cell dysfunction (n=8; n=12) (FIGS. 4C-4E). These results were confirmed by genetic silencing of the pan-αν integrin (CD51) in GSCs using CRISPR/Cas9 (FIG. 4F; FIG. 17). Together, the data support a model in which αν integrins regulate the TGF-β 1 axis involved in GSC-induced NK cell dysfunction (FIG. 4G).

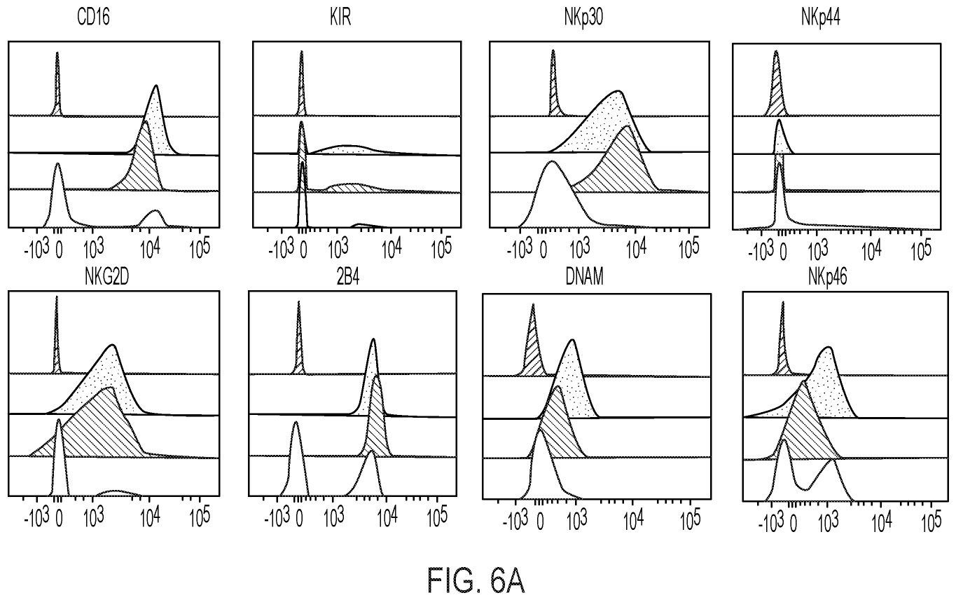

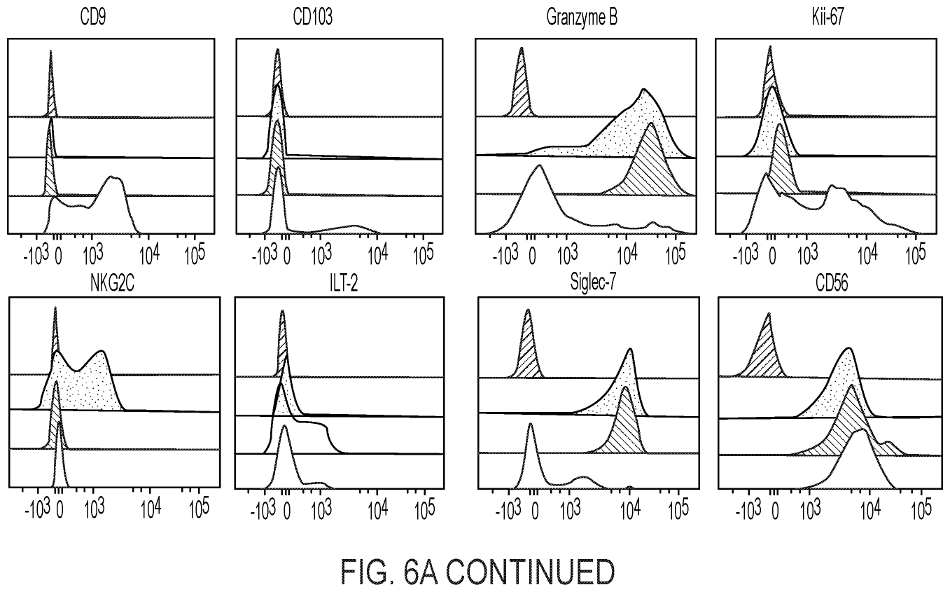

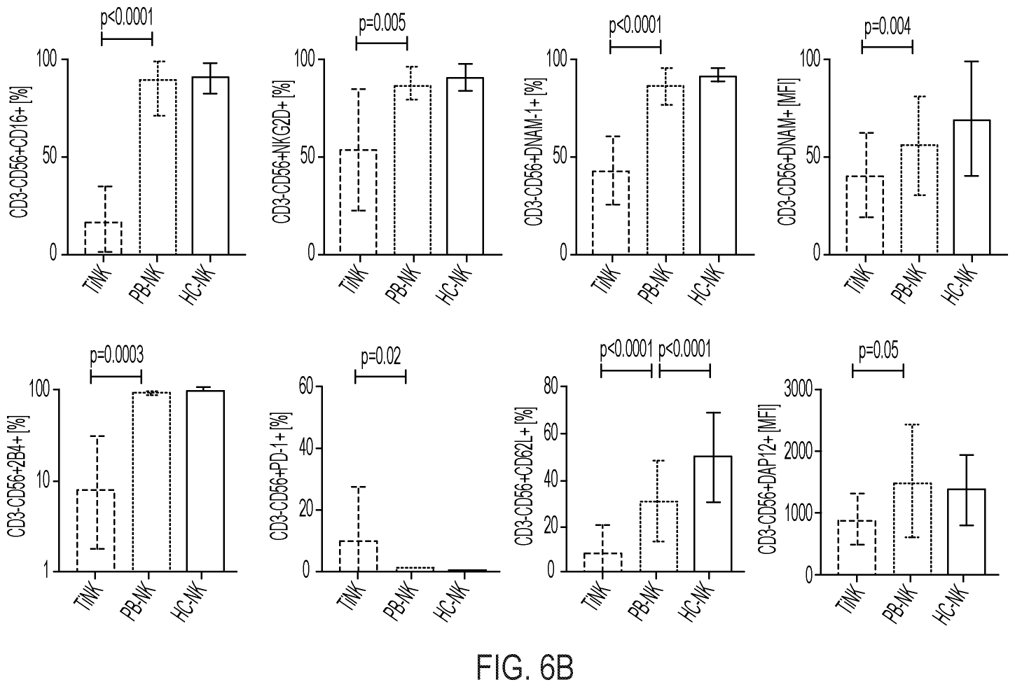

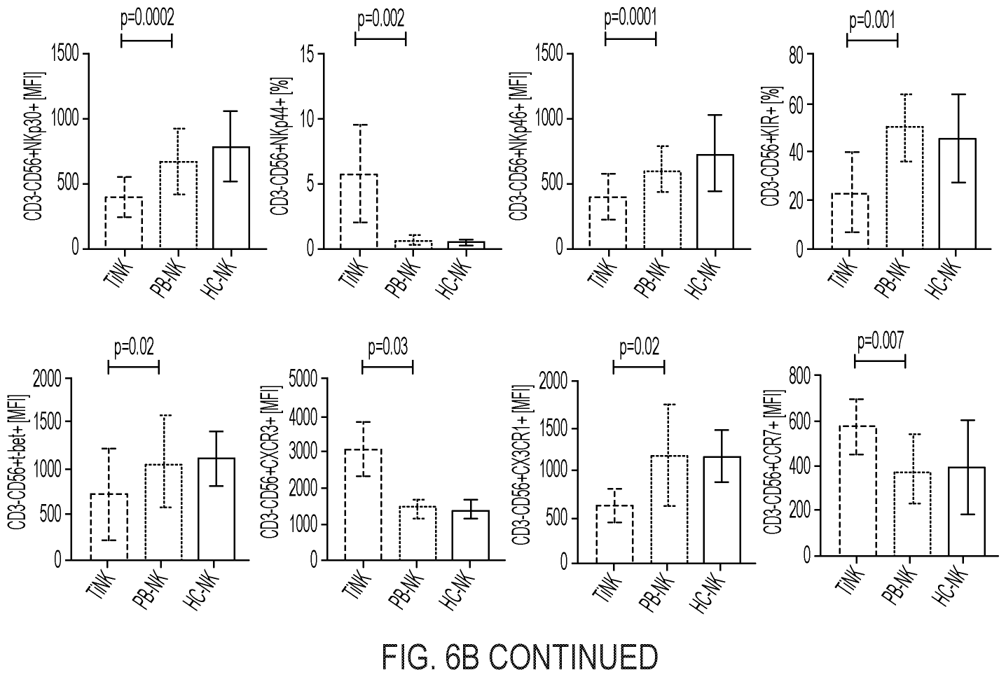

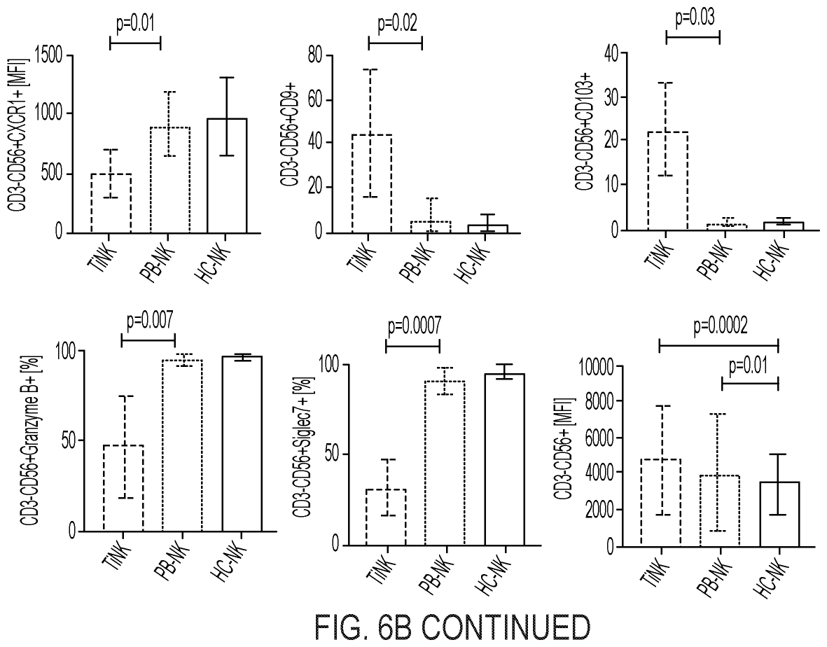

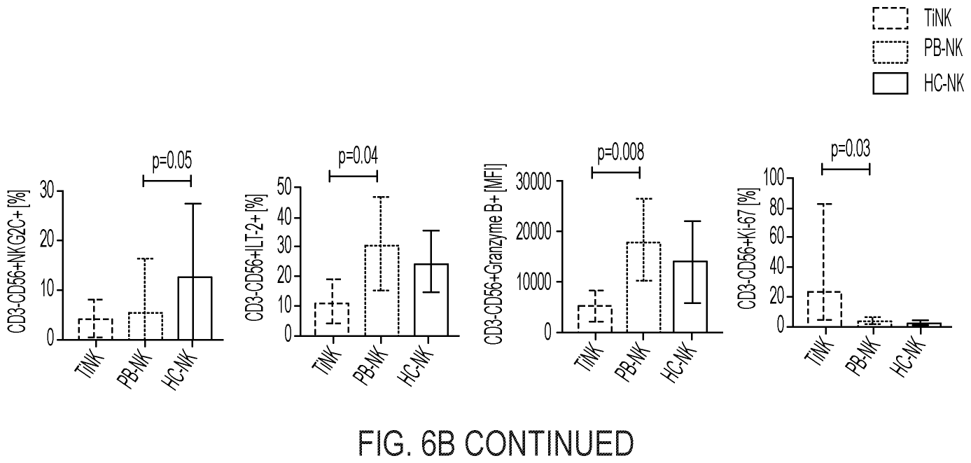

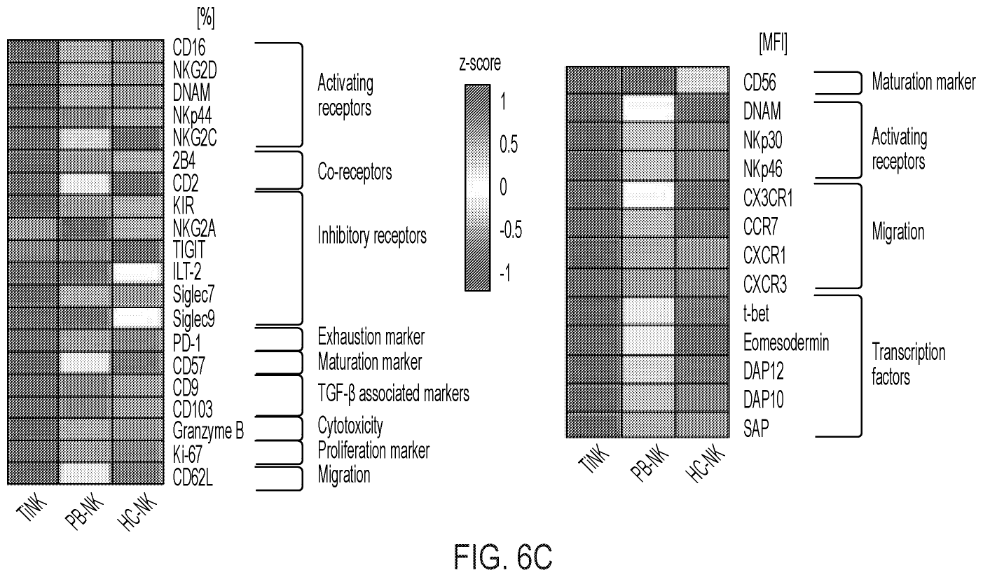

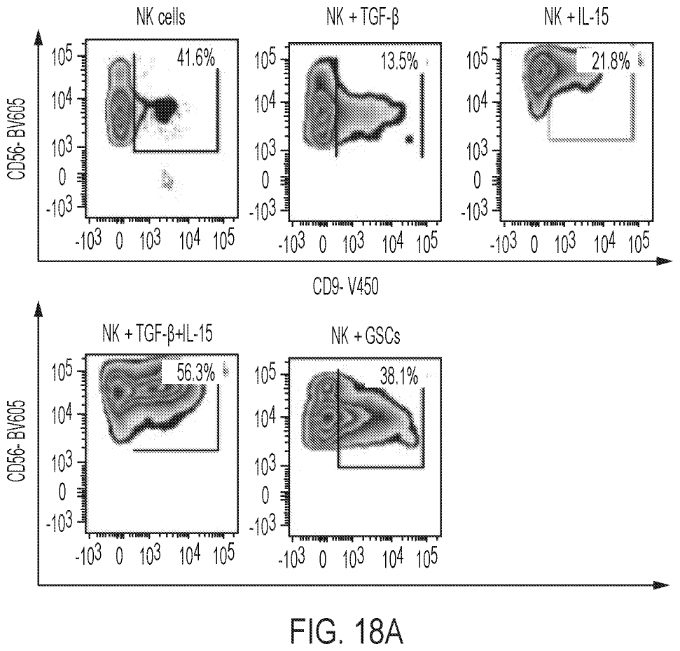

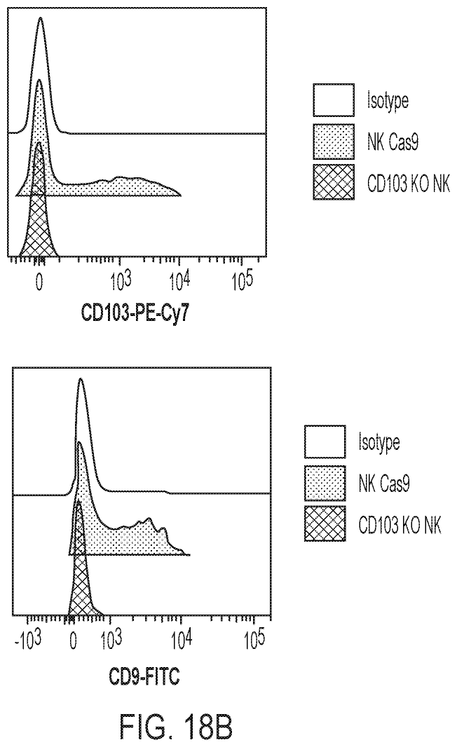

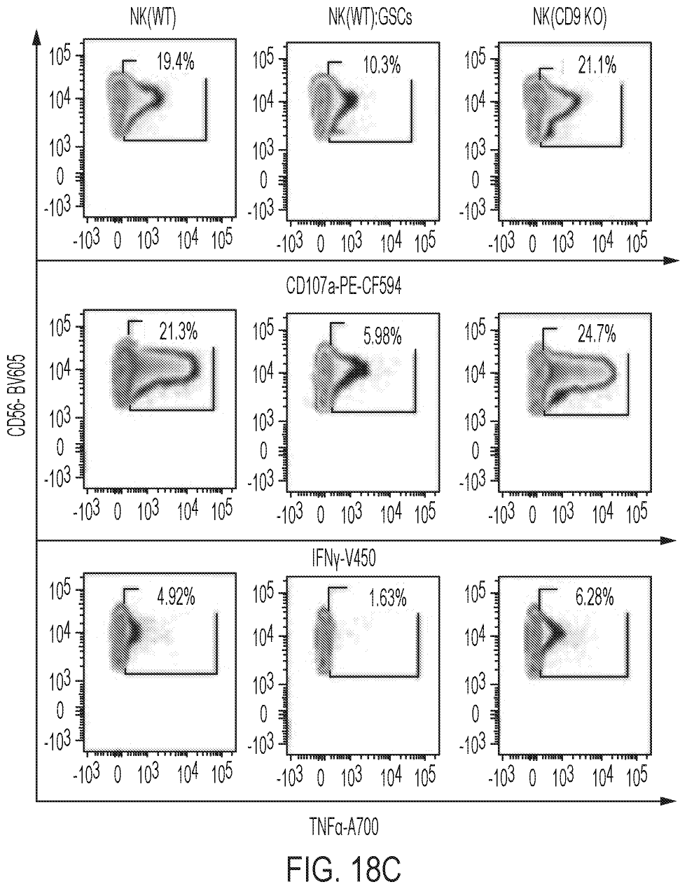

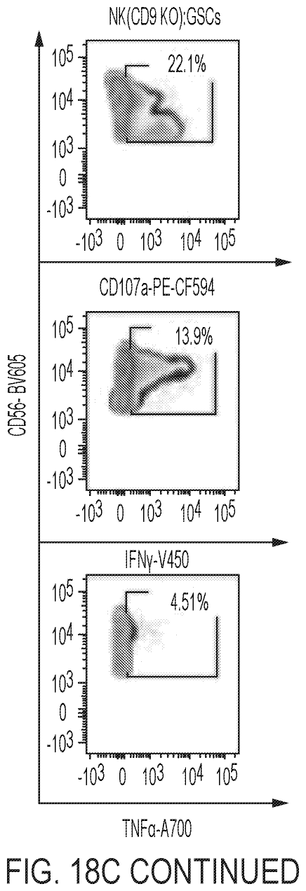

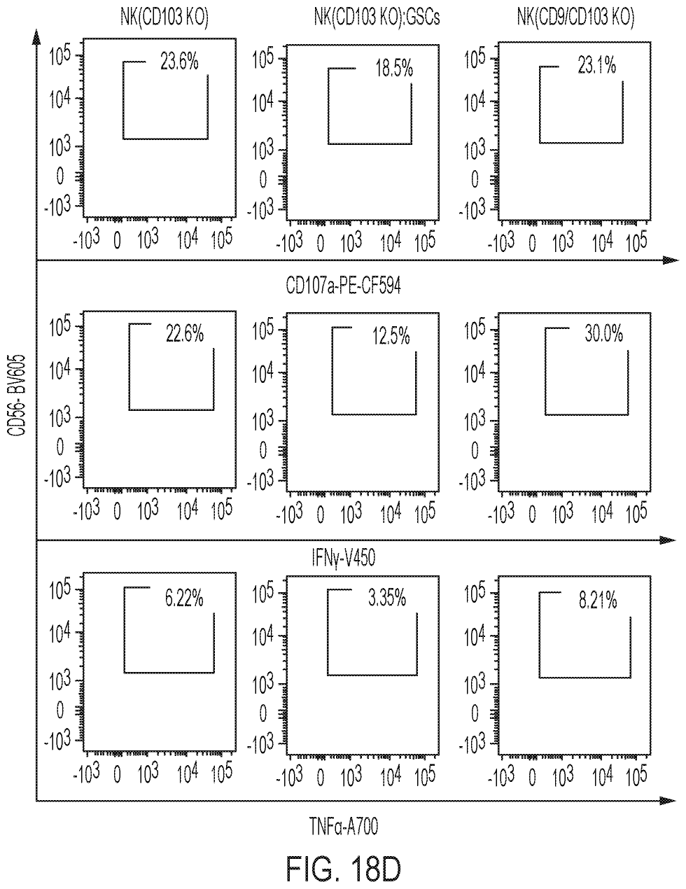

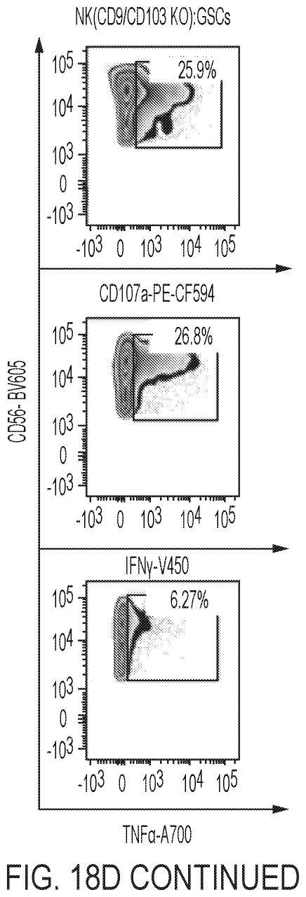

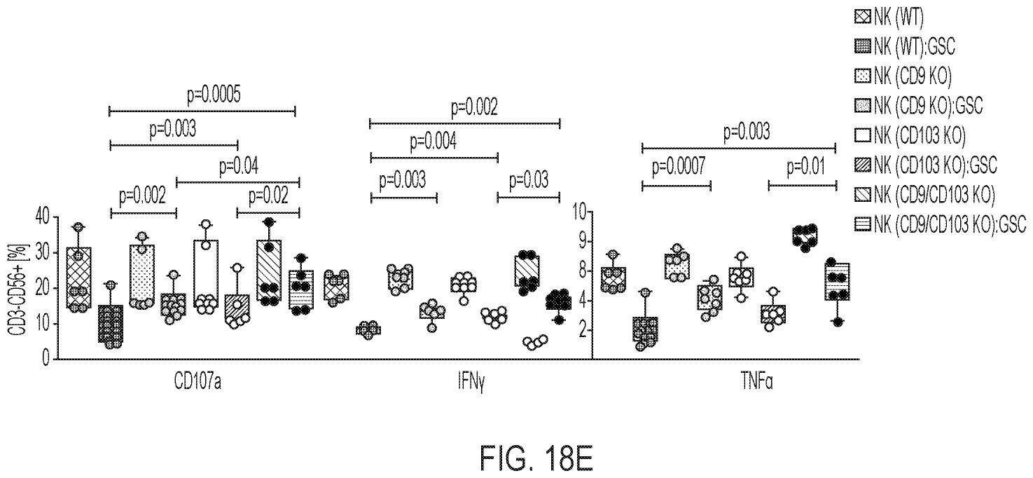

The identity of the surface ligands on NK cells that could potentially interact with αν integrins to mediate GSC-NK cell crosstalk was sought. In addition to binding extracellular matrix components, αν integrins bind tetraspanins, such as CD9, through their active RDG binding site³⁶. Indeed, CD9 and CD103 are upregulated on GBM TiNKs (FIG. 1E; FIG. 6) and can be induced on healthy NK cells after co-culture with TGF-β1 (FIG. 18A). Thus, CRISPR Cas9 gene editing was used to knockout (KO) CD9 and CD103 in healthy donor NK cells (FIG. 18B) and tested the cytotoxicity of wild type (WT, treated with Cas9 only), CD9 KO, CD103 KO or CD9/CD103 double KO NK cells after co-culture with GSCs. As shown in FIGS. 18C-18E, silencing of either CD9 or CD103 resulted in partial improvement in the cytotoxic function of NK cells co-cultured with GSCs by comparison with WT control. In contrast, CD9/CD103 double KO NK cells co-cultured with GSCs retained their cytotoxicity against K562 targets. This suggests that αν integrins on GSCs bind CD9 and CD103 on NK cells to regulate the TGF-β1 axis involved in GSCinduced NK cell dysfunction.

### Example 10

Inhibition of the αν Integrin TGF-β1 Axis Enhances NK Cell Anti-Tumor Activity In Vivo

The mechanistic insights gained from the above studies suggest that the αν integrin-TGF-β1 axis regulates an important evasion tactic used by GSCs to suppress NK cell cytotoxic activity and therefore may provide a useful target for immunotherapy of high-grade GBM. To test this prediction, a PDX mouse model of patient-derived GSC was used, in which, ffLuc+ patient-derived GSCs (0.5×10⁶) were stereotactically implanted on day 0 through a guide-screw into the right forebrain of NOD/SCID/IL2Rγc null mice (n=4-5 per group). After 7 days, the mice were treated intratumorally with 2.0×10⁶ human NK cells every 7 days for 11 weeks (FIG. 5A) with either galunisertib to block the TGF-β signaling or cilengitide to block the integrin pathway. Galunisertib was administered five times a week by oral gavage and cilengitide three times a week by intraperitoneal injection. Animals implanted with tumor that were either untreated or received NK cells alone, galunisertib alone or cilengitide alone served as controls.

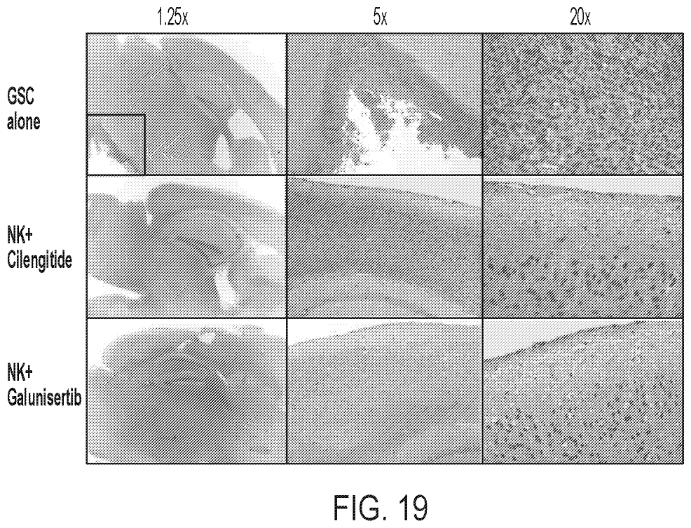

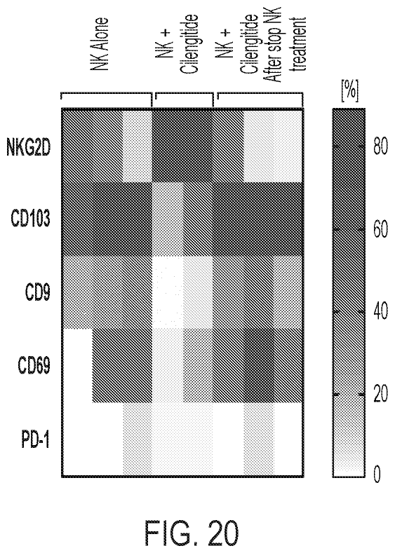

As shown in FIG. 5B, tumor bioluminescence rapidly increased in untreated mice and in mice that were either untreated or treated with the monotherapies cilengitide, galunisertib or NK cells. By contrast, adoptive NK cell transfer combined with cilengitide or galunisertib treatment led to significant improvements in tumor control (p<0.0001) (FIG. 5B-C) and survival for each comparison (p=0.005) (FIG. 5D). Moreover, no evidence of tissue damage or meningoencephalitis was noted in mice treated with human allogeneic PBderived NK cells plus cilengitide or galunisertib (FIG. 19). In animals that received adoptive NK cell infusion combined with either cilengitide or galunisertib, TiNKs harvested after mice were sacrificed showed a higher expression of NKG2D and reduced levels of CD9 and CD103 (FIG. 20).

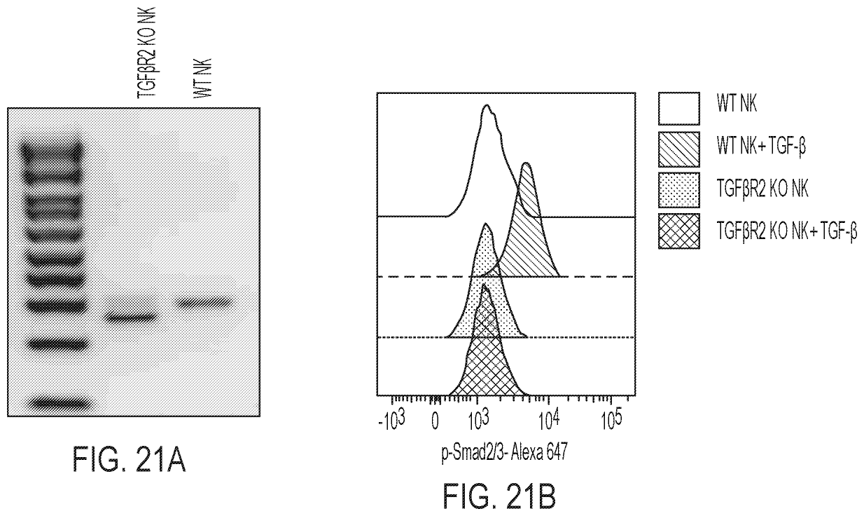

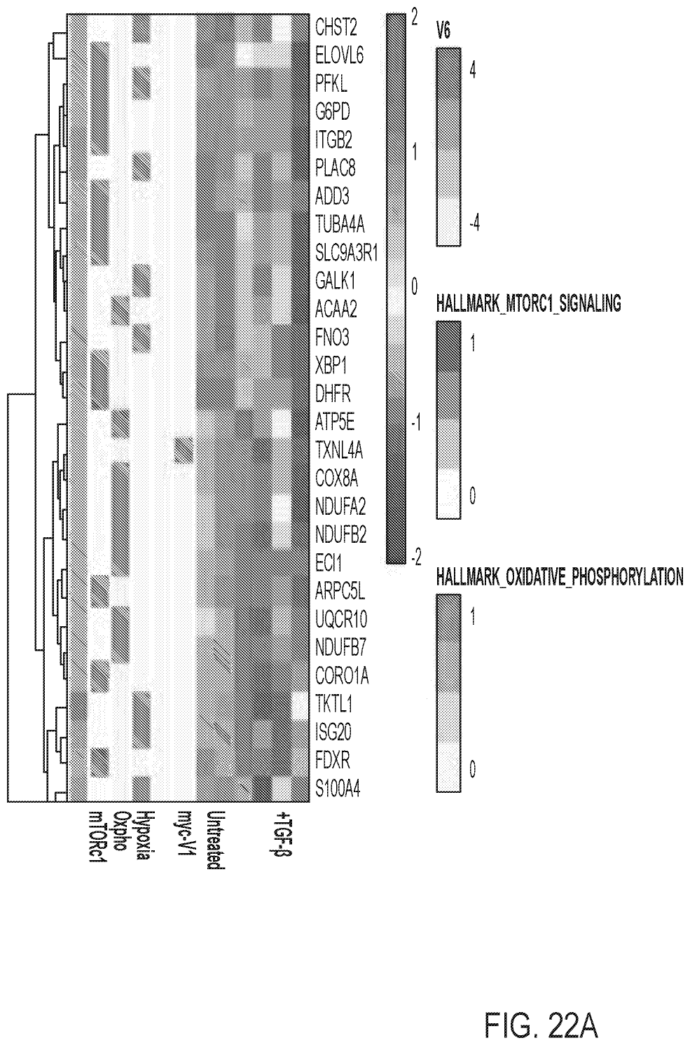

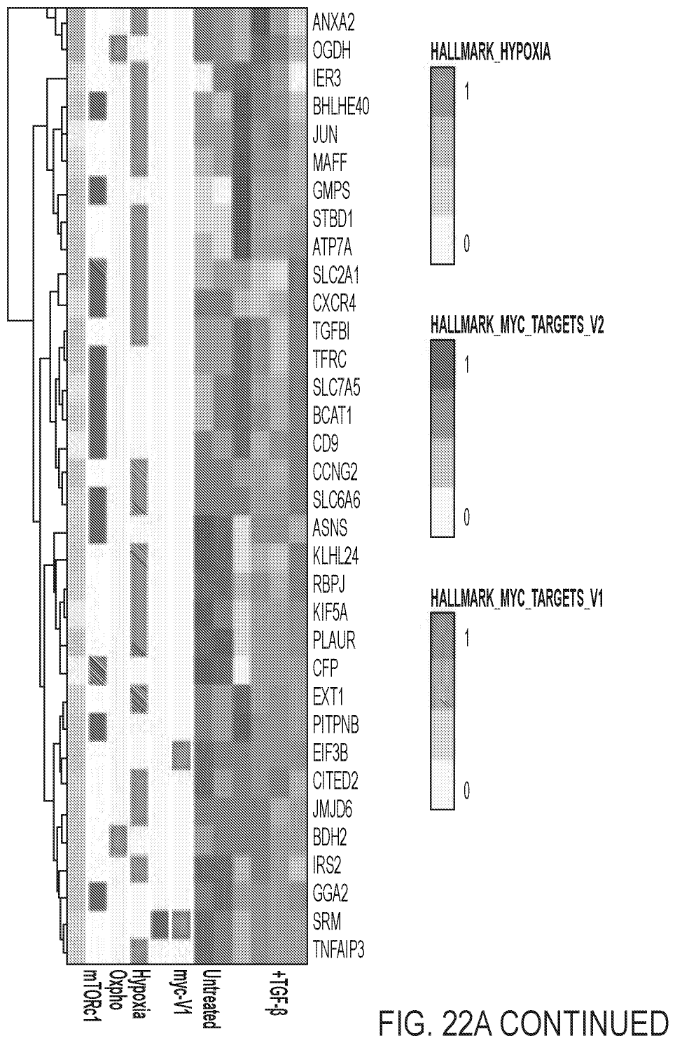

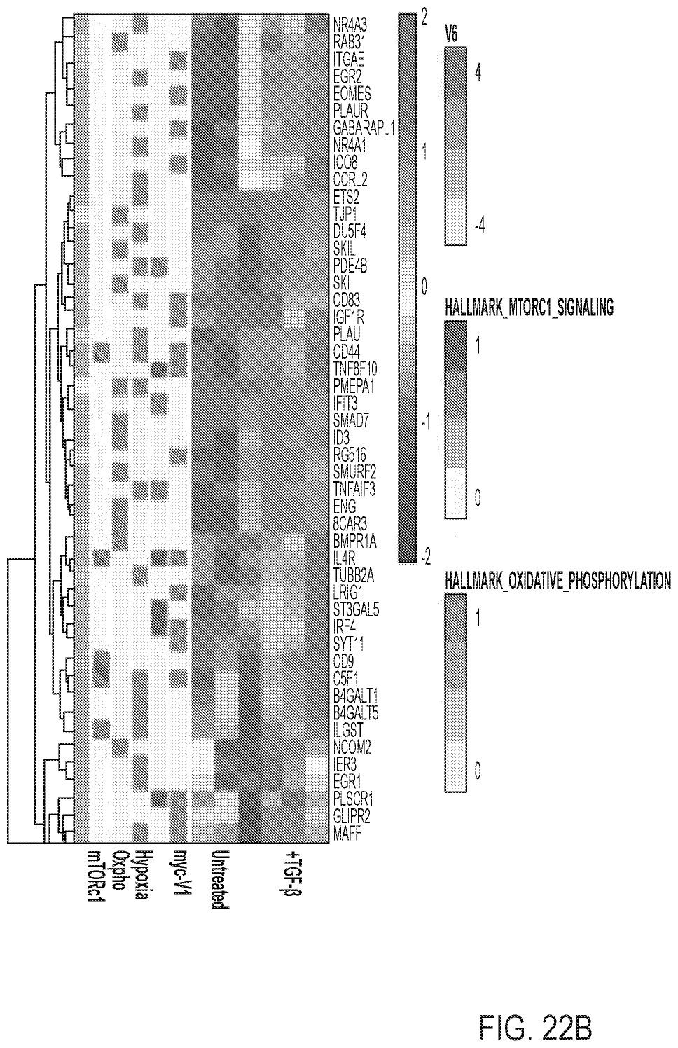

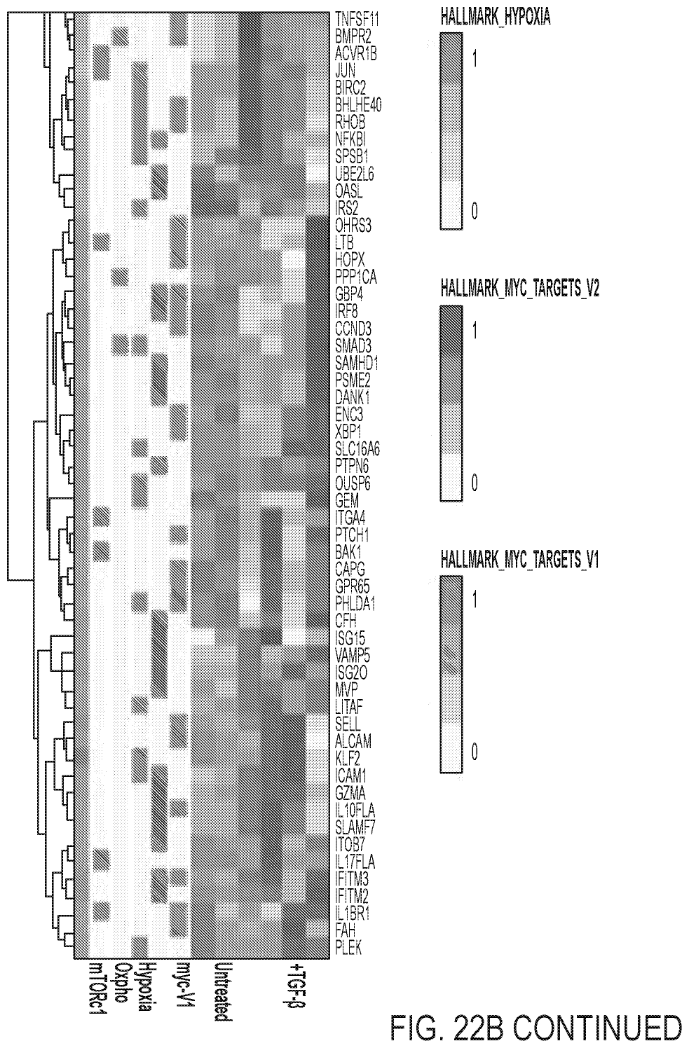

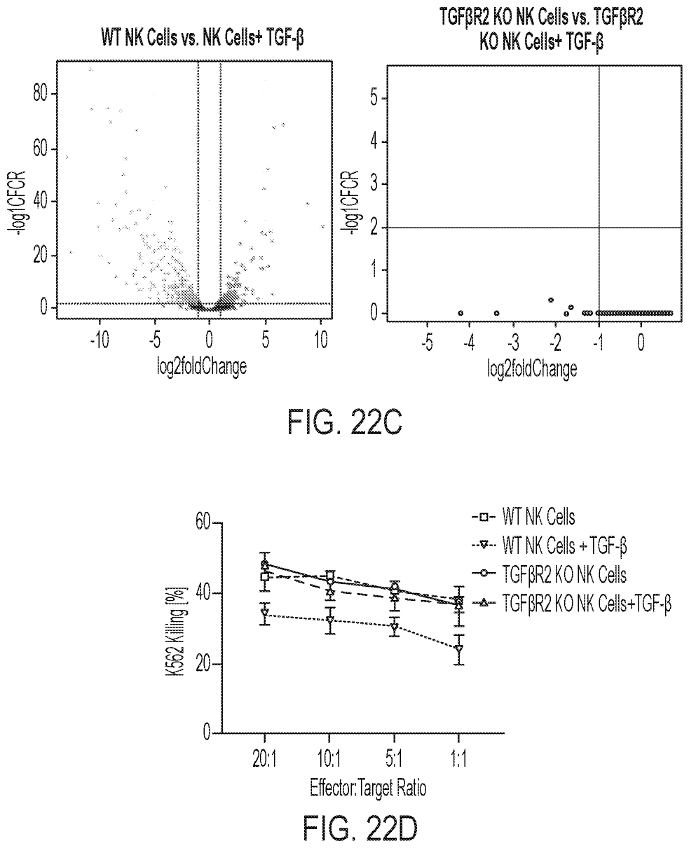

Finally, the impact of KO of TGFβR2 using CRISPR Cas9 gene editing (FIG. 21) was tested on GSC-induced NK cell dysfunction. In vitro, TGFβR2 KO NK cells treated with 10 ng/ml of recombinant TGF-β for 48 hours maintained their phenotype compared to wild-type controls as demonstrated by mass cytometry analysis (FIGS. 5E-5F), transcriptomic analysis (FIGS. 22A-22C) and cytotoxicity against K562 targets (FIG. 5G; FIG. 22D). Next, the in vivo anti-tumor activity of TGFβR2 KO NK cells was analyzed by treating mice intracranially at day 7 post tumor implantation with either WT NK cells, WT NK cells plus galunisetib, or TGFβR2 KO NK cells followed by subsequent NK cell injections every 4 weeks through a guide screw (FIG. 5H). Tumor bioluminescence increased rapidly in untreated mice, while adoptive transfer of WT NK cells in combination with 5×per week galunisertib or TGFβR2 KO NK cells led to significant tumor control as measured by bioluminescence imaging (FIGS. 5I-5J). In conclusion, the data support a combinatorial approach of NK cell adoptive therapy together with disruption of the αν integrin-TGF-β1 axis to target GBM.

### Example 11

Improvement of Nk Cell Function Against Gscs

Glioblastoma is among the most deadly and difficult to treat of all human cancers. This difficulty can be in part attributed to the presence of GSCs that differ from their mature progeny in numerous ways, including resistance to standard chemotherapy and radiotherapy, and the ability to initiate tumors and mediate recurrence following treatment. Thus, unless the GSCs within the high-grade GBM tumors are eliminated, the possibility of cure is unlikely. Here, it was shown that NK cells can readily kill GSCs in vitro and can infiltrate these tumors. Yet, they display an altered phenotype with impaired function within the tumor microenvironment, indicating that GSCs have evolved mechanisms to evade NK cell immune surveillance.

Studies to test this hypothesis showed that the underlying mechanism for GSC-induced NK cell dysfunction relies on cell-cell contact between NK cells and GSCs, resulting in the subsequent release and activation of TGF-β, a potent immunosuppressive cytokine that plays a critical role in suppressing the immune response37. One model of this protective mechanism is summarized in FIG. 4G. Based on the results, it is likely that disruption of the blood-brain barrier caused by the tumor allows the migration of NK cells into the GBM tumor tissue. Once in the tumor, NK cells interact with GSCs. This results in the release and production of TGF-β by GSCs in a cell-cell contact-dependent manner through interactions between αν integrins on GSCs and various ligands on NK cells such as CD9 and CD103. TGF-β is then cleaved from its latent complex form to its biologically active form by proteases such as MMP-2 and MMP-9, released mostly by GSCs. The release of these matrix metalloprotease is further driven by αν integrins and by TGF-β itself, as shown by data presented here and by others^{38,39,40,41,42,43,44}. TGF-β, in turn, suppresses NK cell function by inducing changes in their phenotype, transcription factors, cytotoxic molecules and chemokines. These modifications render NK cells irreversibly incapable of killing GSCs.

An important aspect of this model is the cross-talk between the αν integrins on GSCs and the TGFβ-induced receptors CD9 and CD103 on NK cells as the main mediators of TGF-β production and subsequent NK cell dysfunction. Silencing the pan-αν integrin (CD51) in GSCs by CRISPR/Cas9 gene editing or pharmacologic inhibition with cilengitide prevented GSC-induced NK cell dysfunction, diminished Smad⅔ phosphorylation and decreased TGF-β production in co-cultures of GSCs and NK cells. The αν integrins have been proposed to modulate latent TGF-β activation through two different mechanisms: (i) an MMP-dependent mechanism based on the production of MMP2 and MMP9 by glioma cells and GSCs, but not healthy brain tissue³³, which proteolytically cleave TGF-β from LAP and (ii), an MMP-independent mechanism, that relies on cell traction forces^{38,41,43,44}. This duality may explain why the MMP- 2/9 inhibitors used in this study could only partially protect NK cells from GSC-induced dysfunction. Current therapeutic strategies such as radiation therapy may in fact potentiate this vicious cycle of immune evasion. Indeed, radiation therapy has been shown to promote the growth of the therapy resistant GSCs by upregulating TGF-β and integrin expression⁴⁵. Inhibition of the αν integrin-TGF-β axis may thus be crucial not only for the success of immunotherapeutic strategies but also for that of conventional therapies.

Although a number of small molecules that globally inhibit TGF-β are in development for glioblastoma patients, most have been associated with prohibitive toxicity⁴⁶ and lack of efficiency, as shown using trabedersen, a TGF-02 oligodeoxynucleotide antisense⁴⁷. This could be attributed to the irreversible inhibition of NK cell function through TGF-β released from the GSCs. Since the NK cells have already been adversely affected by the tumor microenvironment, administration of trabedersen would be futile. Given the ubiquitous nature and multiple functions of TGF-β in the central nervous system, the use of NK cells to eliminate GSCs within tumor tissues would benefit from concomitant use of αν integrin inhibitors to block TGF-β signaling by GSCs, such as cilengitide that binds αV03 and αV05 integrins, or gene editing strategies to delete the TGF-βR2 in NK cells and to protect against TGF-β binding and consequent immunosuppression. Either of these strategies can target local immunosuppressive mechanisms and thus would be expected to reduce excessive toxicity.

Finally, on the strength of these findings, the data show viable immunotherapeutic strategies in which third-party NK cells derived from healthy donors are administered in combination with a pan-αν integrin inhibitor or are genetically edited to silence TGF-βR2 to protect them from immunosuppression, thus, enabling them to recognize and eliminate rare tumor cells with stem-like properties such as GSCs.

### Example 12

### Examples of Methods

I. Patients

Forty-six patients with GBM (n=34 primary GBM; n=12 recurrent GBM) and five patients with low-grade glioma (n=2 low-grade oligodendroglioma; n=3 diffuse astrocytoma) were recruited from The University of Texas MD Anderson Cancer Center (MDACC) for phenotypic (n=28), functional studies (n=14) and single cell RNA sequencing analysis (n=10). All subjects gave full informed and written consent under the Institutional Review Board (IRB) protocol number LAB03-0687. All studies were performed in accordance with the Declaration of Helsinki. Buffy coat from normal donors was obtained from gulf Coast Regional Blood Center, Houston, Tex., USA.

II. Sample Processing

Peripheral blood mononuclear cells (PBMCs) were purified with Histopaque (Sigma-Aldrich) by density gradient separation. Freshly resected human glioblastoma tissue was minced into small pieces using a scalpel, dissociated using a Pasteur pipette, and suspended in RPMI 1640 medium containing Liberase TM Research Grade Enzyme (Roche) at a final concentration of 30 μg/ml. The prepared mixture was incubated for 1 hour at 37° C. with agitation. After brief centrifugation, the pellet was resuspended in 20 ml of 1.03 Percoll (GE Healthcare) underlayed with 10 ml of 1.095 Percoll, and overlayed with 10 ml of 5% FBS in PBS (Hyclone). The tube was centrifuged at 1,200 g for 20 minutes at room temperature with no brake. After centrifugation, the cell layer on top of 19 the 1.095 Percoll was collected, filtered through a 70-m nylon strainer (BD Biosciences), washed and cells were counted using a cellometer (Nexelom Bioscience, Lawrence, Mass.). NK cells were magnetically purified using NK cells isolation kit (Miltenyi).

III. Characterization of GBM tumor infiltrating NK cells (TiNKs), peripheral blood NK cells (PB-NK) and healthy control NK cells (HC-NK)

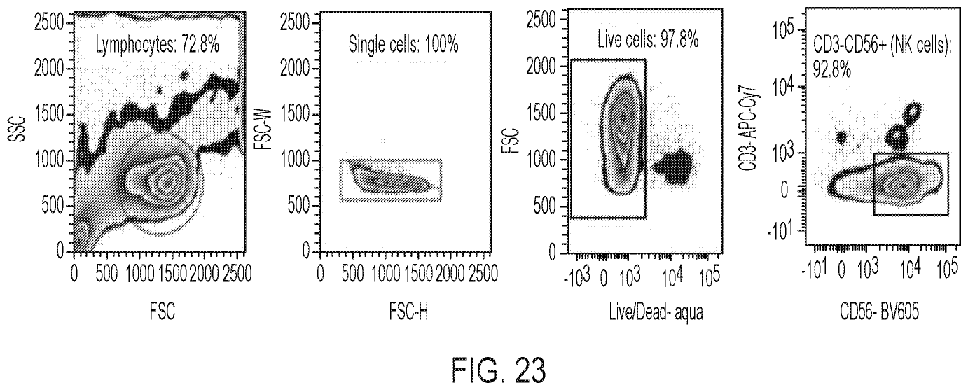

Flow cytometry: Freshly isolated TiNKs, PB—NK and HC-NK cells were incubated for 20 minutes at room temperature with Live/Dead-Aqua (Invitrogen) and the following surface markers: CD2-PE-Cy7, CD3-APC-Cy7, CD56-BV605, CD16-BV650, NKp30-biotin, DNAM-FITC, 2B4-PE, NKG2D-PE, Siglec-7-PE, Siglec-9-PE, PD-1-BV421, CD103-PECy7, CD62L-PE-Cy7, CCR7-FITC, CXCR1-APC, CX3CRi-PE-cy7, CXCR3-PerCP-Cy5.5 (Biolegend), NKp44-PerCP eflour710 and TIGIT-APC (eBiosciences), streptavidin-BV785, PD-1-V450, CD9-V450 and NKp46-BV711 (BD Biosciences), Human KIR-FITC and NKG2C-APC (R&D), NKG2A-PE-Cy7 and ILT2-APC (Beckman Coulter) and CD57-PerCP (Novus Biological). For detection of intracellular markers, cells were fixed/permeabilized using BD FACS lysing solution and permeabilizing solution 2 according to manufacturer's instructions (BD Biosciences) followed by intracellular staining with Ki-67-PE and t-bet-BV711 (Biolegend), Eomesodermin-eFluor660 and SAPPE (eBiosciences), Granzyme-PE-CF594 (BD Biosciences), DAP12-PE (R&D) and DAP10-FITC (Bioss Antibodies) for 30 minutes in room temperature. All data were acquired with BD-Fortessa (BD Biosciences) and analyzed with FlowJo software. The gating strategy for detection of NK cells is presented in FIG. 23.

IV. Mass Cytometry

The strategy for antibody conjugation is described elsewhere⁴⁸. Table 1 shows the list of antibodies used for the characterization of NK cells in the study.

TABLE 1
List of antibodies used for mass cytometry

| | TARGET | Clone | ISOTOPE | Source |
|---|---|---|---|---|
| 1 | CD45 | HI30 | 89Y | Fluidigm |
| 2 | CD57 | HCD57 | 115In | Biolegend |
| 3 | KIR2DL1/S5 | HP-MA4 | 141Pr | Biolegend |
| 4 | EOMES | WD1928 | 142Nd | Thermo Fisher |
| 5 | KIR2DL2/L3 | DX27 | 143Nd | Biolegend |
| 6 | Siglec 7 | EMR8-5 | 144Nd | BD Biosciences |
| 7 | CD62L | DREG-56 | 145Nd | Biolegend |
| 8 | KIR2DL5 | UP-R1 | 146Nd | Miltenyi |
| 9 | CD20 | 2H7 | 147Sm | Fluidigm |
| 10 | TRAIL | RIK-2 | 148Nd | BD Bioscience |
| 11 | SYK | 4D10.2 | 149Sm | Biolegend |
| 12 | KIR2DL4 | 181703 | 150Nd | R&D |
| 13 | CD25 | 2A3 | 151Eu | Miltenyi |
| 14 | CD3Z | 6B10.2 | 152Sm | Biolegend |
| 15 | DAP12 | 406288 | 153Eu | R&D |
| 16 | TIGIT | MBSA43 | 154Sm | Thermo Fisher |
| 17 | CD27 | L128 | 155Gd | Bd Bioscience |
| 18 | KLRG1 | 13F12F2 | 156Gd | Thermo Fisher |
| 19 | CD94 | DX22 | 158Gd | Biolegend |
| 20 | NKP30 | Z5 | 159Tb | Fluidigm |
| 21 | KIR3DL2 | 539304 | 160Gd | R&D |
| 22 | T-BET | 4B10 | 161Dy | Biolegend |
| 23 | NKP46 | BAB281 | 162Dy | Fluidigm |
| 24 | CISH | Polyclonal | 163Dy | R&D |
| 25 | CCR7 | G043H7 | 164Dy | Biolegend |
| 26 | NKG2D | ON72 | 166Er | Beckman Coulter |
| 27 | 2B4 | C1.7 | 167Er | Thermo Fisher |
| 28 | KI67 | Ki67 | 168Er | Biolegend |
| 29 | NKG2A | Z199 | 169Tb | Fluidigm |
| 30 | CD3 | UCHT-1 | 170Er | Biolegend |
| 31 | DNAM | DX11 | 171Yb | BD Bioscience |
| 32 | Perforin | dG9 | 172Yb | Biolegend |
| 33 | Granzyme B | GB11 | 173Yb | BD Bioscience |
| 34 | KIR2DS4 | JJC11.6 | 174Yb | Miltenyi |
| 35 | KIR3DL1 | DX9 | 175Lu | BD Bioscience |
| 36 | CD56 | NCAM16.2 | 176Yb | BD Bioscience |
| 37 | CD16 | 3G8 | 209Bi | Fluidigm |
| | Cisplatin | | 198Pt | Fluidigm |

Briefly, NK cells were harvested, washed twice with cell staining buffer (0.5% bovine serum albumin/PBS) and incubated with 5 μl of human Fc receptor blocking solution (Trustain FcX, Biolegend, San Diego, Calif.) for 10 minutes at room temperature. Cells were then stained with a freshly prepared CyTOF antibody mix against cell surface markers as described previously^{48,49}. Samples were acquired at 300 events/second on a Helios instrument (Fluidigm) using the Helios 6.5.358 acquisition software (Fluidigm). Mass cytometry data were normalized based on EQTM four element signal shift over time using the Fluidigm normalization software 2. Initial data quality control was performed using Flowjo version 10.2. Calibration beads were gated out and singlets were chosen based on iridium 193 staining and event length. Dead cells were excluded by the Pt195 channel and further gating was performed to select CD45+ cells and then the NK cell population of interest (CD3-CD56+). A total of 320,000 cells were proportionally sampled from all samples to perform automated clustering. The mass cytometry data were merged together using Principal Component Analysis (PCA), “RunPCA” function, from R package Seurat (v3). Dimensional reduction was performed using “RunUMAP” function from R package Seurat (v3) with the top 20 principal components. The UMAP plots were generated using the R package ggplot2 (v3.2.1). Data were analyzed using automated dimension reduction including (viSNE) in combination with FlowSOM for clustering⁵⁰ for the deep phenotyping of immune cells as published before⁵¹. Relevant cell clusters were further delineated using an in-house pipeline for cell clustering. To generate the heatmap, CD45+CD56+CD3-gated FCS files were exported from FlowJo to R using function “read.FCS” from the R package flowCore (v3.10). The markers expression was transformed using acrsinh with a cofactor of 5. The mean values of 36 markers were plotted as heat map using the function “pheatmap” from R package pheatmap (v1.0.12). Markers with similar expression were hierarchically clustered.

V. Incucyte Live Imaging

After co-culture with GSCs, NT NK cells and TGFORII KO NK cells were purified and labeled with Vybrant DyeCycle Ruby Stain (ThermoFisher) and co-cultured at a 1:1 ratio with K562 targets labeled with CellTracker Deep Red Dye (ThermoFisher). Apoptosis was detected using the CellEvent Caspase- 3/7 Green Detection Reagent (ThermoFisher). Frames were captured over a period of 24 hrs at 1 hour intervals from 4 separate 1.75×1.29 mm2 regions per well with a 10× objective using IncuCyte S3 live-cell analysis system (Sartorius). Values from all four regions of each well were pooled and averaged across all three replicates. Results were expressed graphically as percent cytotoxicity by calculating the ratio of red and green overlapping signals (count per image) divided by the red signal (counts per image).

VI. Single Cell RNA Sequencing

Gliomas were mechanically dissociated with scissors while suspended in Accutase solution (Innovative Cell Technologies, Inc.) at room temperature and then serially drawn through 25-, 10- and 5-mL pipettes before being drawn through an 18 ½-gauge syringe. After 10 minutes of dissociation, cells were spun down at 420× g for 5 minutes at 4° C. and then resuspended in 10 mL of a 0.9N sucrose solution and spun down again at 800× g for 8 minutes at 4° C. with the brake off. Once sufficient samples were accumulated to be run in the 10× pipeline (10x Genomics; 6230 Stoneridge Mall Road, Pleasanton, Calif. 94588), cells were then thawed and resuspended in 1 mL of PBS containing 1% BSA, for manual counting. Cells were then stained with the CD45 antibody (BD Biosciences, San Jose, Calif., cat #: 555482) at 1:5 for 20 minutes on ice. Samples had Sytox blue added just before sorting so that only live CD45+ cells would be collected. Cells were then sorted in a solution of 50% FBS and 0.5% BSA in PBS, spun down, and resuspended at a concentration of 700-1200 cells/μL for microfluidics on the 10× platform (10x Genomics). The 10× protocol, which is publicly available, was followed to generate the cDNA libraries that were sequenced. (https://assets.ctfassets.net/an68im78xiti/2NaoOhmA0jot0ggwcyEKaC/fc58451fd97d9cb e12c0abbb097cc38/CG000204_ChromiumNextGEMSingleCell3_v3.1_Rev_C.pdf). libraries were sequenced on an Illumina next-seq 500, and up to 4 indexed samples were multiplexed into one output flow cell using the Illumina high-output sequencing kit (V2.5) in paired-end sequencing (R1, 26nt; R2, 98nt, and i7 index 8nt) as instructed in the 10× Genomics 3′ Single-cell RNA sequencing kit. The data were then analyzed using the cellranger pipeline (10x Genomics) to generate gene count matrices. The mkfastq argument (10x Genomics) was used to separate individual samples with simple csv sample sheets to indicate the well that was used on the i7 index plate to label each sample. The count argument (10x Genomics) was then used with the expected number of cells for each patient. The numbers varied between 2,000 and 8,000 depending on the number of viable cells isolated. Sequencing reads were aligned with GRCh38. The aggr argument (10x Genomics) was then used to aggregate samples from each patient for further analysis. Once gene-count matrices were generated, they were read into an adapted version of the Seurat pipeline19,20 for filtering, normalization, and plotting. Genes that were expressed in less than three cells were ignored, and cells that expressed less than 200 genes or more than 2500 genes were excluded, to remove potentially poor—and high-PCR artifact cells, respectively. Finally, to generate a percentage of mitochondrial DNA expression and to exclude any cells with more than 25% mitochondrial DNA (as these may be doublets or low-quality dying cells), cells were normalized using regression to remove the percent mitochondrial DNA variable via the scTransform2l command which corrects for batch effects as well. Datasets were then processed for principal component analysis (PCA) with the RunPCA command, and elbow plots were printed with the ElbowPlot command in order to determine the optimal number of PCs for clustering; 15 PCs were chosen for this analysis.

Next, the cell clusters were identified and visualized using SNN and UMAP, respectively, before generating a list of differentially-expressed genes for each cluster. A list of differentially-expressed genes was generated to label the clusters at low resolution (0.1). These clusters' labels were based on at least three differentially-expressed genes, and violin plots were generated to show the relative specificity to the cluster. Differentially-expressed genes were identified using cutoffs for min.pct=0.25 and log fc.threshold=0.25. Plots were generated with either the DimPlot, FeaturePlot or VlnPlot commands. Next clusters containing NK cell populations were identified in both the PBMC and GBM dataset: NK markers included KLRD1, NKG7, and NKTR. Analyses performed on the combination of PBMC and GBM NK cells, were joined using the FindIntegrationAnchors command to determine genes that can be used to integrate the two datasets-after the determination of the Anchors the IntegrateData command were used to combine the two datasets. Data were then normalized using the scTransform command. Datasets were then processed for PCA with the RunPCA command, and elbow plots were printed with the ElbowPlot command in order to determine the optimal number of PCs for clustering, 15 PCs were chosen for this analysis. Next, the cell clusters were identified and visualized using SNN and UMAP, respectively, before generating a list of differentially-expressed genes for each sample. Plots were generated with either the DimPlot, FeaturePlot or VlnPlot commands.

VII. GSC Culture

GSCs were obtained from primary human GBM samples as previously described^{52,53}. Patients gave full informed and written consent under the IRB protocol number LAB03-0687. The GSCs were cultured in stem cell-permissive medium (neurosphere medium): Dulbecco's Modified Eagle Medium containing 20 ng/ml of epidermal growth factor and basic fibroblast growth factor (all from Sigma-Aldrich), B27 (1:50; Invitrogen, Carlsbad, Calif.), 100 units/ml of penicillin and 100 mg/ml streptomycin (Thermo Fisher Scientific, Waltham, Mass.) and passaged every 5-7 days⁵⁴. All generated GSC cell lines used in this paper were generated at MD Anderson Cancer Center and referred to as MDA-GSC.

VIII. NK Cell Expansion

NK cells were purified from PBMCs from healthy donors using an NK cell isolation kit (Miltenyi Biotec, Inc., San Diego, Calif., USA). NK cells were stimulated with irradiated (100 Gy) K562-based feeder cells engineered to express 4-1BB ligand and CD137 ligand (referred to as Universal APC) at a 2:1 feeder cell: NK ratio and recombinant human IL-2 (Proleukin, 200 U/ml; Chiron, Emeryville, Calif., USA) in complete CellGenix GMP SCGM Stem Cell Growth Medium (CellGenix GmbH, Freiburg, Germany) on day 0. After 7 days of expansion, NK cells were used for in vivo mice experiments and for in vitro studies.

IX. Characterization of GSCs and Human Astrocytes

Human fetal astrocytes cell lines were purchased from Lonza (CC-2565) and Thermo Fisher Scientific (N7805100) and the human astroglia cell line (CRL-8621) was purchased from the American Type Culture Collection (ATCC). The cells were separated into single cell suspension using accutase (Thermo Fisher Scientific) for GSCs and trypsin for the attached astrocytes. The cells were then stained for MICA/B-PE, CD155-PE-Cy7, CD112-PE, HLA-E-PE and HLA-ABC-APC (Biolegend), ULBP1-APC, ULBP2/5/6-APC and ULBP3-PE (R&D), HLA-DR (BD Biosciences) and B7-H6-FITC (Bioss antibodies) for 20 minutes before washing and acquiring by flow cytometry.

X. NK Cell Cytotoxicity Assay

NK cells were co-cultured for 5 hours with K562 or GSCs target cells at an optimized effector:target ratio of 5:1 together with CD107a PE-CF594 (BD Biosciences), monensin (BD GolgiStopTM) and BFA (Brefeldin A, Sigma Aldrich). NK cells were incubated without targets as the negative control and stimulated with PMA (50 ng/mL) and ionomycin (2 mg/mL, Sigma Aldrich) as positive control. Cells were collected, washed and stained with surface antibodies (mentioned above), fixed/permeabilized (BD Biosciences) and stained with IFN-7 v450 and TNF-α Alexa700 (BD Biosciences) antibodies.

XI. Chromium Release Assay

NK cell cytotoxicity was assessed using chromium (siCr) release assay. Briefly, K562 or GSCs target cells were labeled with siCr (PerkinElmer Life Sciences, Boston, Mass.) at 50 μCi/5×105 cells for 2 hours. ⁵¹Cr-labeled K562/GSC targets (5×105) were incubated for 4 h with serially diluted magnetically isolated NK cells in triplicate. Supernatants were then harvested and analysed for ⁵¹Cr content.

XII. Suppression Assay

For studies of NK cell suppression by GSCs and human astrocytes, magnetically selected healthy NK cells were cultured in Serum-free Stem Cell Growth Medium (SCGM; CellGro/CellGenix) supplemented with 5% glutamine, 5 μM HEPES (both from GIBCO/Invitrogen), and 10% FCS (Biosera) in 96-well flat-bottomed plates (Nunc) at 100,000/100D11. NK cells were co-cultured either alone (positive control) or with GSCs or astrocytes at a 1:1 ratio for 48 hours at 37° C. before performing functional assays to assess NK cells cytotoxicity.

XIII. Functional Assays for Blocking NK Cytotoxicity

Magnetically purified NK cells were cultured alone or with blocking antibodies against NKG2D (clone 1D11), DNAM (clone 11A8) and NKp30 (clone P30-15) (Biolegend) overnight (5 μg/ml). ⁵¹Cr release assay was then performed as described above. For HLA-KIR blocking, GSCs were cultured alone or with an HLA-ABC blocking antibody (clone W 6/32, Biolegend) before performing ⁵¹Cr release assay.

XIV. NK Cell Functional Assays

GSCs and purified NK cells were co-cultured for 48 hours in the presence of anti-TGFβ 123 (5 μg/ml) (R&D), HLA-ABC blocking antibody (clone W 6/32, Biolegend), CD44 blocking antibody (clone IM7, Biolegend), ILT-2 (CD85J) blocking antibody (clone HP-F1, ThermoFisher), CD155 blocking antibody (clone D171, GenTex), CD112 blocking antibody (clone TX31, Biolegend), 10 μM LY2109761, 10 μM galunisertib (LY2157299), 10 μM Cilengitide (Cayman Chemical) or 1 μM MMP-2/MMP-9 inhibitor I (Millipore). Cytotoxicity assays were then performed as described above.

XV. Transwell Assays

NK cells (1×105) were either added directly to GSCs at a ratio of 1:1 or placed in transwell chambers (Millicell, 0.4 m; Millipore) for 48 hours at 37° C. After 48 hours, cultured cells were harvested to measure NK cell cytotoxicity by both ⁵¹Cr release assay and cytokine secretion assay.

XVI. NK Cell Recovery Assays

NK cells were cultured either with GSCs in a 1:1 ratio or alone for 48 hours. After 48 hours of co-incubation, NK cells were then either purified again by bead selection and resuspended in SCGM media or remained in culture with GSCs for an additional 48 hours and then used for ⁵¹Cr release assay. In a second assay, after reselection, NK cells were cultured for another 5 days in SCGM in the presence of 5 ng/ml IL-15 with or without 10 μM galunisertib before use for ⁵¹Cr release assay.

XVII. TGF-β ELISA and MMP 2/9 Luminex

NK cells and GSCs were either co-cultured or cultured alone for 48 hours in serum free SCGM growth medium. After 48 hours, supernatants were collected and secretion of TGF R and MMP2/3/9 was assessed in the supernatant by TGFβ1 ELISA kit (R&D systems) or MMP2/3/9 luminex kit (eBiosciences) as per the manufacturer's protocol.

XVIII. Reverse Transcriptase-Polymerase Chain Reaction (RT-PCR), and quantitative Real-time PCR (qPCR)

RNA was isolated using RNeasy isolation kit (Qiagen). A 1 μg sample of total RNA was reverse transcribed to complementary DNA using the iScript cDNA Synthesis Kit (Bio-Rad) according to the manufacturer's instructions. Then, an equivalent volume (1 μL) of complementary DNA (cDNA) was used as a template for quantitative real-time PCR (qPCR) and the reaction mixture was prepared using iTaqTM Universal SYBR® Green Supermix (Biorad) according to the manufacturer's instructions. Gene expression was measured in a StepOnePlusTM (Applied Biosystem) instrument according to the manufacturer's instruction with the following gene-specific primers: TGFB1 (forward, 5′—AACCCACAACGAAATCTATG-3′ (SEQ ID NO:10); reverse, 5′-CTTTTAACTTGAGCCTCAGC-3′ (SEQ ID NO:11)); and 18S (forward, 5′-AACCCGTTGAACCCCATT-3′ (SEQ ID NO:12); reverse, 5′-CCATCCAATCGGTAGTAGCG-3′ (SEQ ID NO:13)). The gene expression data were quantified using the relative quantification (AACt) method, and 18S expression was used as the internal control.

XIX. CRISPR Gene Editing of Primary NK Cells and GSCs

crRNAs to target CD9, CD103 and CD51 were designed using the Integrated DNA Technologies (IDT) predesigned data set. Guides with the highest on-target and off-target scores were selected. The crRNA sequences are reported in Table 2.

TABLE 2
Sequences used to target CD9, CD103,
CD51 and TGFβR2 genes using CRISPR-Cas9
gene editing

| | | | Gene |
|---|---|---|---|
| | Sequence | Exon | Name |
| CD9 | GAATCGGAGCCATAGTCCAA | 2 | CD9 |
| crRNA | (SEQ ID NO: 14), PAM:TGG | | |
| CD103 | GCATTCAAGTGCTGGTCCGG | 5 | ITGAE |
| crRNA | (SEQ ID NO: 15), PAM:CGG | | |
| CD51 | CCACGTCTAGGTTGAAGGCG | 1 | ITGAV |
| crRNA | (SEQ ID NO: 16), PAM:CGG | | |
| TGFβRII | GACGGCTGAGGAGCGGAAGA | 5 | TGFBR2 |
| gRNA | (gRNA1; (SEQ ID NO: 17)) | | |
| | TGTGGAGGTGAGCAATCCCC | | |
| | (gRNA2; (SEQ ID NO: 18)) | | |

crRNAs were ordered from IDT (www.idtdna.com/CRISPR-Cas9) in their proprietary Alt-R format. Alt-R crRNAs and Alt-R tracrRNA were re-suspended in nuclease-free duplex buffer (IDTE) at a concentration of 200 μM. Equal amount from each of the two RNA components was mixed together and diluted in nuclease-free duplex buffer at a concentration of 44 μM. The mix was boiled at 95° C. for 5 minutes and cooled down at room temperature for 10 minutes. For each well undergoing electroporation, Alt-R Cas9 enzyme (IDT cat #1081058, 1081059) was diluted to 36 μM by combining with resuspension buffer T at a 3:2 ratio. The guide RNA and Cas9 enzyme were combined using a 1:1 ratio from each mixture. The mixture was incubated at room temperature for 10-20 minutes. Either a 12 well plate or a 24 well plate was prepared during the incubation period. This required adding appropriate volume of media and Universal APCs (1:2 ratio of effector to target cells) supplemented with 200 IU/ml of IL-2 (for NK cells only) into each well. Target cells were collected and washed twice with PBS. The supernatant was removed as much as possible without disturbing the pellet and the cells were resuspended in Resuspension Buffer T for electroporation. The final concentration for each electroporation was 1.8 μM gRNA, 1.5 μM Cas9 nuclease and 1.8 μM Cas9 electroporation enhancer. The cells were electroporated using Neon Transfection System, at 1600V, 10 ms pulse width and 3 pulses with 10ul electroporation tips (Thermo Fisher Scientific (cat # MPK5000)). After electroporation the cells were transferred into the prepared plate and placed in the 37C incubator. The knockout efficiency was evaluated using flow cytometry 7 days after electroporation. Anti-CD51-PE antibody (Biolegend) was used to verify KO efficacy in GSCs.

To knockout TGFβR2, two sgRNA guides (Table 2) spanning close regions of exon 5 were designed and ordered from IDT; 1 μg cas9 (PNA Bio) and 500 ng of each sgRNA were incubated on ice for 20 minutes. After 20 minutes, NK cells 250,000 were added and re-suspended in T-buffer to a total volume of 14ul (Neon Electroporation Kit, Invitrogen) and electroporated before transfer to culture plate with APCs as described above.

XX. Phospho-Smad⅔ Assay

NK cells were stained with Live/dead-aqua and CD56 ECD (Beckman Coulter) for 20 min in the dark at RT, washed with PBS fixed for 10 min in the dark. After one wash, the cells were permeabilized (Beckman Coulter kit) and stained with p-(5465/5467)-Smad2/p-(5423/5425)/Smad3-Alexa 647 mAb Phosflow antibody (BD Biosciences) for 30 minutes at room temperature. Cells incubated with 10 ng/ml recombinant TGF-β for 45 minutes in 37° C. were used as positive control.

XXI. MMP2 and MMP9 Intracellular Staining and Western Blotting

NK cells and GSCs were either cultured alone, in a transwell chamber or together in the presence or absence of TGF-β blocking antibodies (R&D) for 48 hours. BFA was added for the last 12 hours of culture. Cells were then fixed/permeabilized (BD Biosciences) and stained with anti-MMP2-PE (R&D) and MMP9-PE (Cell Signalling) for 30 minutes before acquisition of data by flow cytometry. The surface markers CD133, CD3 and CD56 were used to distinguish NK cells and GSCs for data analysis.

XXII. Xenogeneic Mouse Model of GBM

To assess the anti-tumor effect of NK cells against GSCs in vivo, a NOD/SCID IL-2R7null (NSG) human xenograft model (Jackson Laboratories, Bar Harbor, Me.) was used. Intracranial implantation of GSCs into male mice was performed as previously described⁵⁵. A total of 60 mice were used. 0.5×10⁵ GSCs were implanted intracranially into the right frontal lobe of 5 week old NSG mice using a guide-screw system implanted within the skull. To increase uniformity of xenograft uptake and growth, cells were injected into 10 animals simultaneously using a multiport Microinfusion Syringe Pump (Harvard Apparatus, Holliston, Mass.). Animals were anesthetized with xylazine/ketamine during the procedure. For in vivo bioluminescent imaging, GSCs were engineered to express luciferase by lentivirus transduction. Kinetics of tumor growth was monitored using weekly bioluminescence imaging (BLI; Xenogen-IVIS 200 Imaging system; Caliper, Waltham, Mass.). Signal quantitation in photons/second (p/s) was performed by determining the photon flux rate within standardized regions of interest (ROI) using Living Image software (Caliper). 2x106 in 3 l expanded donor peripheral blood NK cells⁵⁶ were injected intracranially via the guide-screw at day 7 post tumor implantation, and then every 7 days for 11 weeks. Mice were treated with either cilengitide or galunisertib (both from MCE Med Chem Express, Monmouth Junction, NJ) in the presence or absence of intracranial NK cell injection. Cilengitide was administered intraperitoneally 3 times a week starting at day 1 (250 μg/100 μl PBS) while galunisertib was administered orally (75 mg/kg) by gavage 5 days a week starting at day 1 (see FIG. 5A). In a second experiment, mice were injected intracranially via the guide screw 7 days post tumor inoculation with either wild type (WT) NK cells, WT NK cells plus galunisertib or TGFβR2 KO NK cells followed by subsequent NK cells injections every 4 weeks as describe above. Mice that presented neurological symptoms (i.e. hydrocephalus, seizures, inactivity, and/or ataxia) or moribund were euthanized. Brain tissue was then extracted and processed for NK cells extraction. All animal experiments were performed in accordance with recommendations in the Guide for the Care and Use of Laboratory Animals of the National Institute of Health, and approved by the Institutional Animal Care and Use Committee (IACUC) protocol number 00001263-RN01 at MD Anderson Cancer Center.

XXIII. Mice Brain Tissue Processing and Analysis

Brain tissue from the animals was collected and NK cells were isolated using a percoll (GE Healthcare) gradient fallowing protocol described by Pino et al⁵⁷. Briefly, brain tissue was dissociated using a 70 μm cell strainer (Life Science, Durham, N.C.). Cell suspension was re suspended in a 30% isotonic percoll solution and layer on top of a 70% isotonic percoll solution. Cells were centrifuged at 500 G for 30 minutes and 18oC with no brake. 2-3 ml of the 70%-30% interface was collected in a clean tube and washed with PBS 1X. After this procedure, cells were ready for immunostaining with mouse CD45, human CD45, CD56, CD3, CD103, CD9, CD69, PD-1 and NKG2D all from Biolegend.

XXIV. Histopathology

Brain tissue specimens from untreated control mice, mice treated with either NK cells alone, cilengitide alone, galunisertib alone or with combination therapy of NK+ cilengitide or NK+ galunisertib were collected. The specimens were bisected longitudinally and half of each brain was fixed in 10% neutral buffered formalin and were then embedded in paraffin. Formalin-fixed, paraffin embedded tissues were sectioned at 4 pm, and stained routinely with hematoxylin and eosin. Brains were examined for the presence or absence of glioblastoma tumor cells. Sections lacking tumor were also evaluated for evidence of meningoencephalitis using a Leica DM 2500 light microscope by a board-certified veterinary pathologist. One section was examined from each sample. Representative images were captured from comparable areas of cerebral hemispheres with a Leica DFC495 camera using 1.25×, 5×, and 20× objectives.

XXV. Statistical Analyses

Statistical significance was assessed with the Prism 6.0 software (GraphPad Software, Inc.), using unpaired or paired two-tailed t-tests as appropriate. For survival comparison a Log-rank test was used. Graphs represent mean and standard deviation (SD). For comparison of bioluminescence among the treatment groups ANOVA was used. A P≤0.05 was considered to be statistical significance.

### Example 13

Genetic Engineering to Protect Nk Cells from Tumor-Mediated and Iatrogenically-Induced Immune Suppression

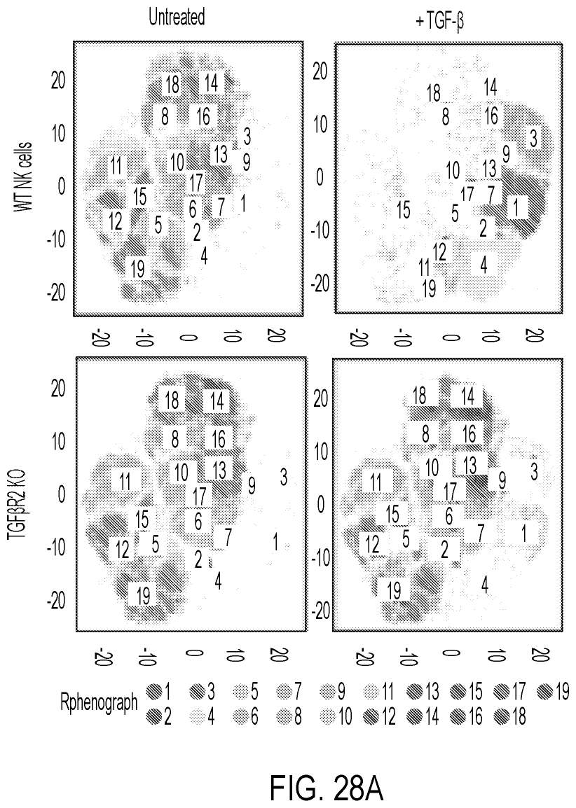

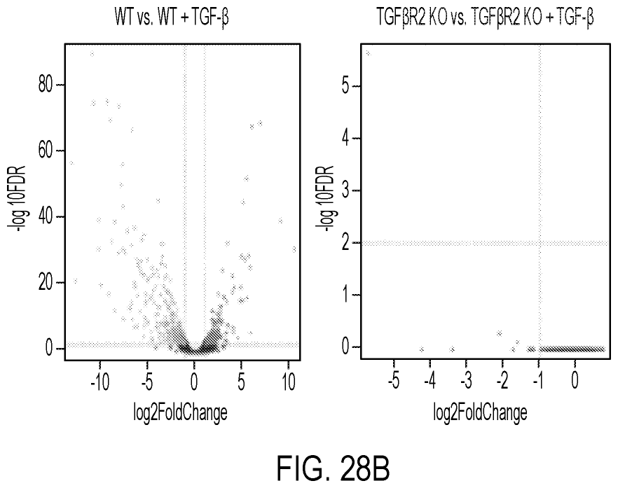

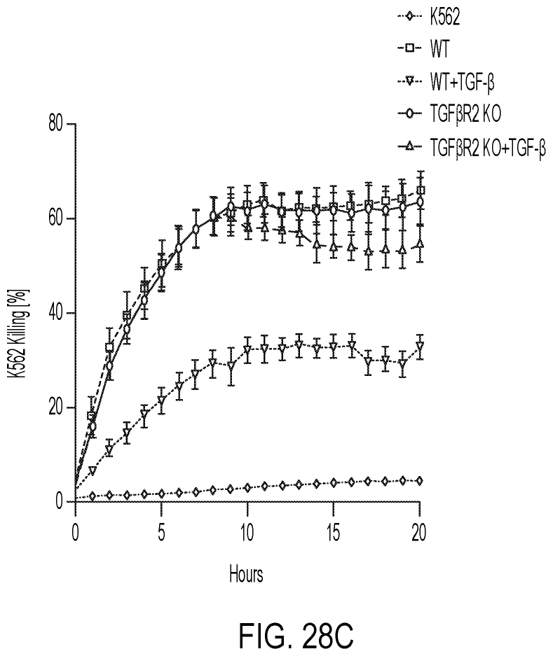

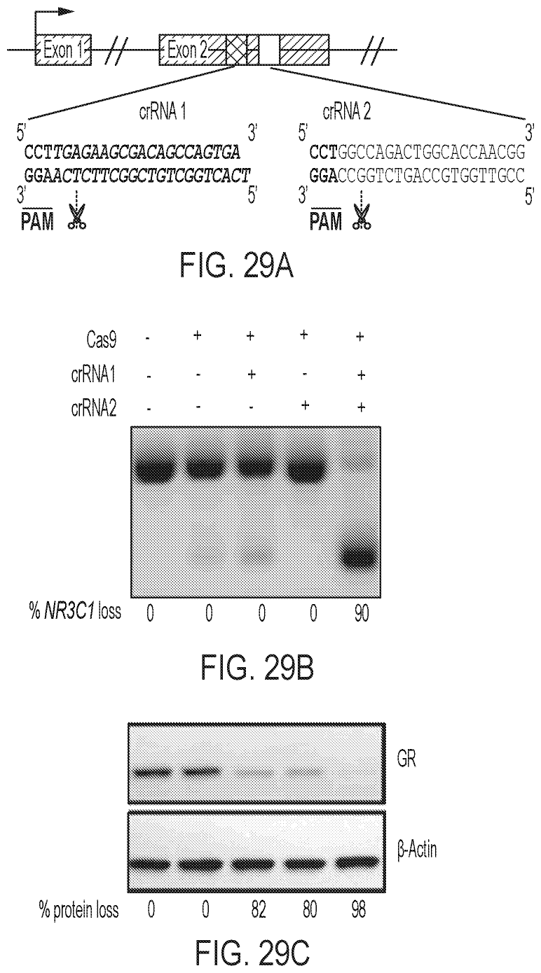

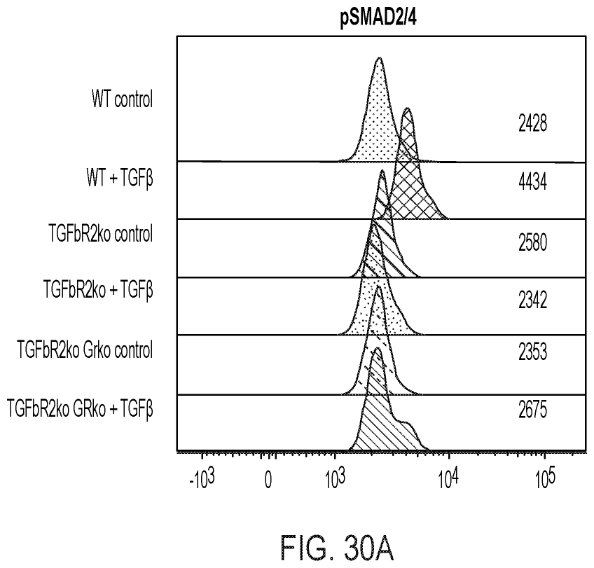

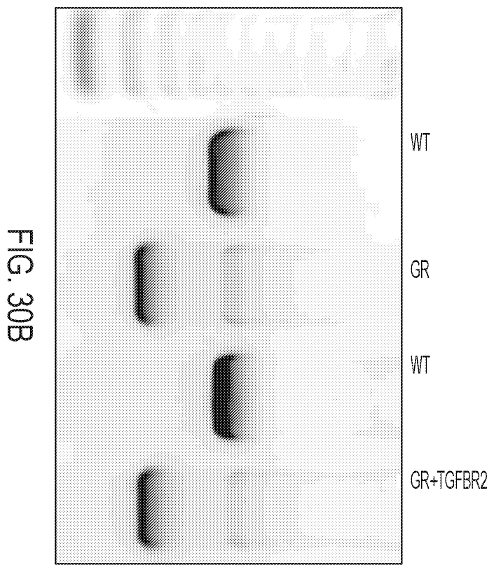

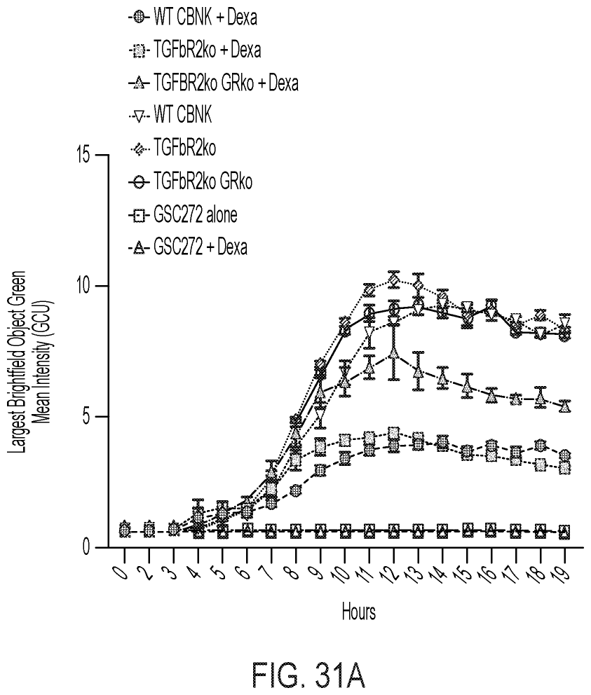

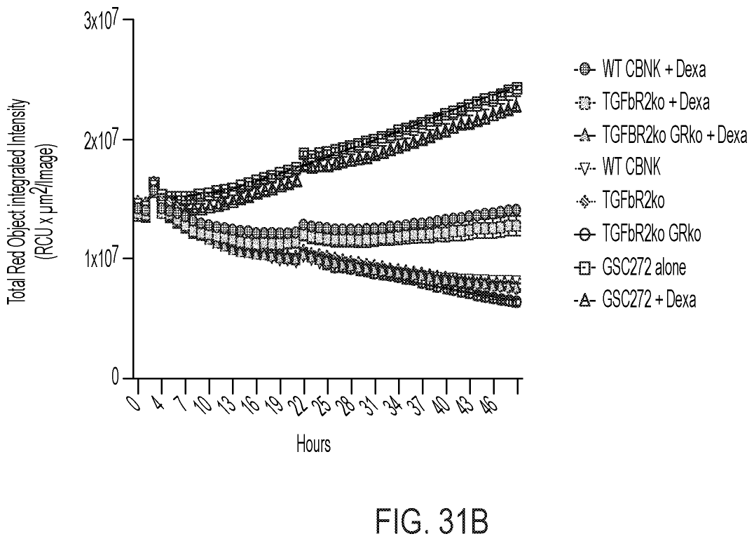

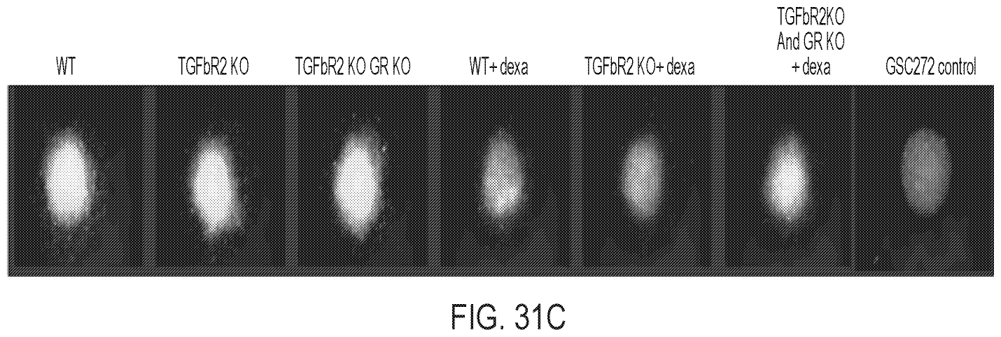

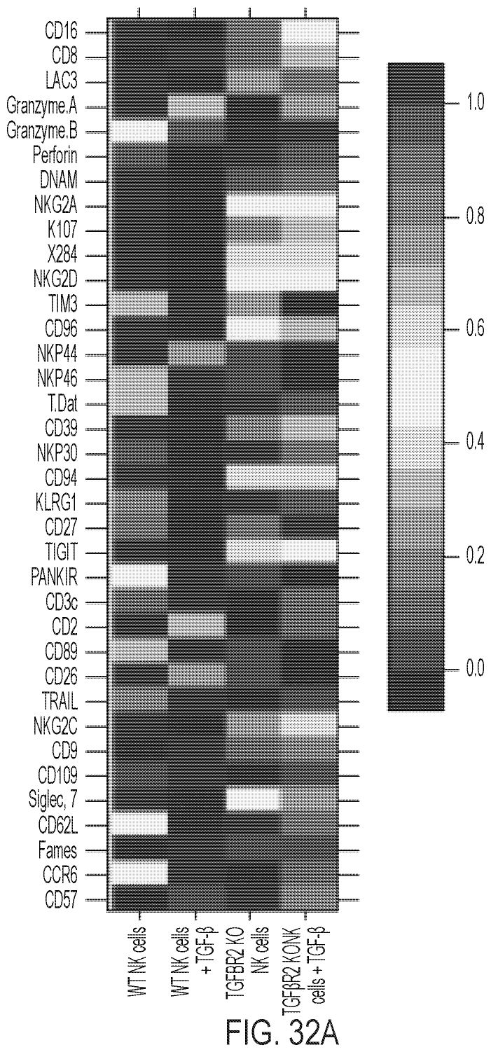

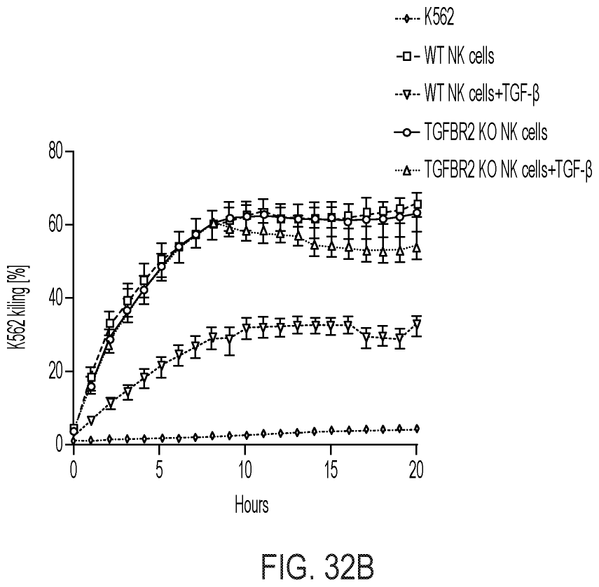

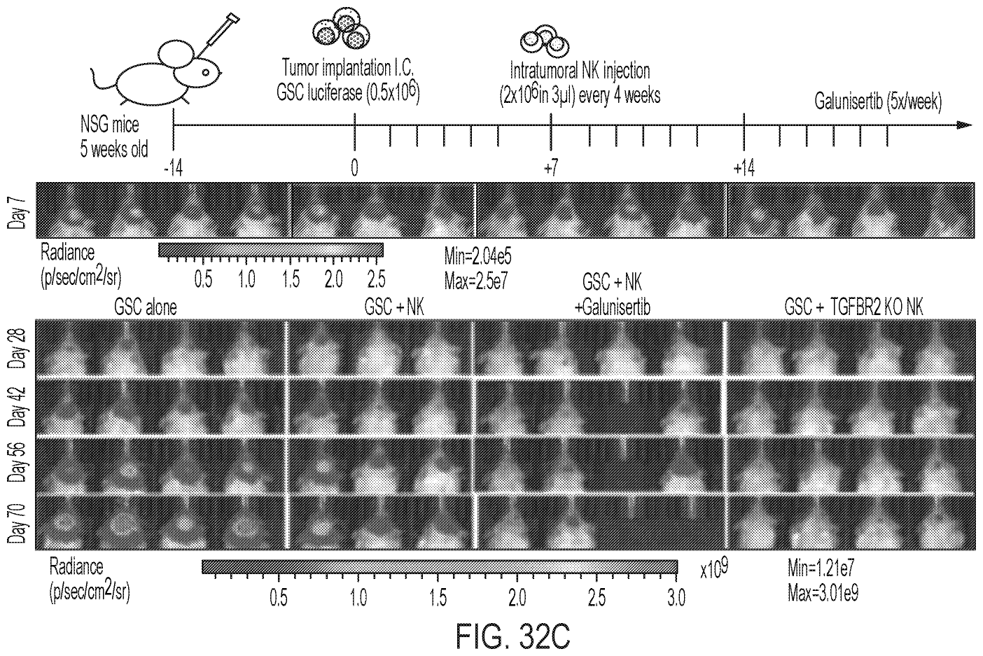

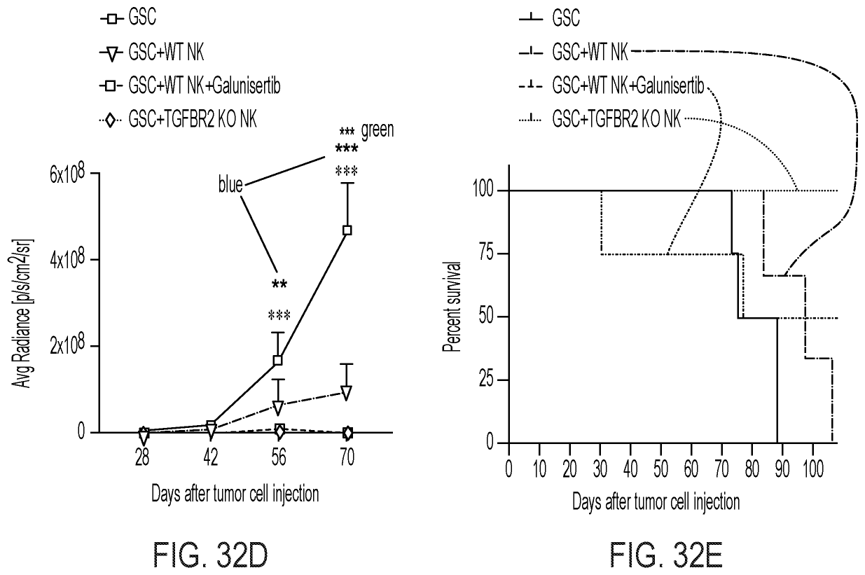

A limitation of cell therapy in glioblastoma is the use of corticosteroids administered to decrease edema and counter symptoms of raised intracranial pressure and/or adverse events. Corticosteroids are lymphocytotoxic and significantly limit the efficacy of immune cell-based therapies. Thus, in order to protect NK cells from both TGF-β-mediated and corticosteroid-induced immune suppression, we have developed a novel multiplex Cas9 gene-editing approach that allows for the dual deletion of TGF-β receptor 2 (by CRISPR knockout [KO] of exon 5 of the TGF-/βR2 gene), and of the glucocorticoid receptor (GR) (by targeting exon 2 of the NR3C1 gene). In vitro, TGFβR2 KO NK cells treated with 10 ng/ml of recombinant TGF-β for 48 hours showed only minimal changes in their phenotype compared to wild-type controls as demonstrated by mass cytometry analysis, transcriptomic analysis and cytotoxicity against K562 targets (FIG. 28). Similarly, NR3C1 was efficiently silenced (>90%) in NK cells as determined by PCR and western blot analysis (FIG. 29). Next, it was shown that the dual KO NK cells (TGF-βR2-/NR3C1) exert impressive anti-tumor activity against GSCs (FIGS. 30-31) and that TGF-βR2-NK cells are highly effective in a GSC PDX mouse model (FIG. 32). These data indicate that a dual gene manipulation strategy can be used to enhance the cytotoxicity of CB-NK cells against GBM by increasing their resistance to the TME and to iatrogenically-induced immunosuppression.

### REFERENCES

The following references, to the extent that they provide exemplary procedural or other details supplementary to those set forth herein, are specifically incorporated herein by reference.
U.S. Pat. No. 5,399,363
U.S. Pat. No. 5,466,468
U.S. Pat. No. 5,543,158
U.S. Pat. No. 5,580,579
U.S. Pat. No. 5,629,001
U.S. Pat. No. 5,641,515
U.S. Pat. No. 5,725,871
U.S. Pat. No. 5,739,169
U.S. Pat. No. 5,756,353
U.S. Pat. No. 5,780,045
U.S. Pat. No. 5,792,451
U.S. Pat. No. 5,801,005
U.S. Pat. No. 5,804,212
U.S. Pat. No. 5,824,311
U.S. Pat. No. 5,830,880
U.S. Pat. No. 5,846,945
U.S. Pat. No. 6,613,308
Austin-Ward and Villaseca, Revista Medica de Chile, 126(7):838-845, 1998.
Bukowski et al., Clinical Cancer Res., 4(10):2337-2347, 1998.
Christodoulides et al., Microbiology, 144(Pt 11):3027-3037, 1998.
Davidson et al., J. Immunother., 21(5):389-398, 1998.
Hanibuchi et al., Int. J. Cancer, 78(4):480-485, 1998.
Hellstrand et al., Acta Oncologica, 37(4):347-353, 1998.
Hollander, Front. Immun., 3:3, 2012.
Hui and Hashimoto, Infection Immun., 66(11):5329-5336, 1998.
Mathiowitz et al., 1997.
Qin et al., Proc. Natl. Acad. Sci. USA, 95(24):14411-14416, 1998.
1. Stupp, R. et al. Radiotherapy plus concomitant and adjuvant temozolomide for glioblastoma. N Engl J Med 352, 987-996 (2005).
2. Ray, S., Bonafede, M. M. & Mohile, N. A. Treatment Patterns, Survival, and Healthcare Costs of Patients with Malignant Gliomas in a Large US Commercially Insured Population. Am Health Drug Benefits 7, 140-149 (2014).
3. Bao, S. et al. Glioma stem cells promote radioresistance by preferential activation of the DNA damage response. Nature 444, 756-760 (2006).
4. Lan, X. et al. Fate mapping of human glioblastoma reveals an invariant stem cell hierarchy. Nature 549, 227-232 (2017).
5. Weiss, T., Weller, M., Guckenberger, M., Sentman, C. L. & Roth, P. NKG2D-Based CAR T Cells and Radiotherapy Exert Synergistic Efficacy in Glioblastoma. Cancer research 78, 1031-1043 (2018).
6. Castriconi, R. et al. NK cells recognize and kill human glioblastoma cells with stem cell-like properties. J Immunol 182, 3530-3539 (2009).
7. Kozlowska, A. K. et al. Differentiation by NK cells is a prerequisite for effective targeting of cancer stem cells/poorly differentiated tumors by chemopreventive and chemotherapeutic drugs. J Cancer 8, 537-554 (2017).
8. Kozlowska, A. K., Kaur, K., Topchyan, P. & Jewett, A. Novel strategies to target cancer stem cells by NK cells; studies in humanized mice. Front Biosci (Landmark Ed) 22, 370-384 (2017).
9. Haspels, H. N., Rahman, M. A., Joseph, J. V., Gras Navarro, A. & Chekenya, M. Glioblastoma Stem-Like Cells Are More Susceptible Than Differentiated Cells to Natural Killer Cell Lysis Mediated Through Killer Immunoglobulin-Like Receptors-Human Leukocyte Antigen Ligand Mismatch and Activation Receptor-Ligand Interactions. Front Immunol 9, 1345 (2018).
10. Daher, M. & Rezvani, K. Next generation natural killer cells for cancer immunotherapy: the promise of genetic engineering. Curr Opin Immunol 51, 146-153 (2018).
11. Rezvani, K. & Rouce, R. H. The Application of Natural Killer Cell Immunotherapy for the Treatment of Cancer. Front Immunol 6, 578 (2015).
12. Nduom, E. K., Weller, M. & Heimberger, A. B. Immunosuppressive mechanisms in glioblastoma. Neuro-oncology 17 Suppl 7, vii9-viil4 (2015).
13. Razavi, S. M. et al. Immune Evasion Strategies of Glioblastoma. Front Surg 3, 11 (2016).
14. Gieryng, A., Pszczolkowska, D., Walentynowicz, K. A., Rajan, W. D. & Kaminska, B. Immune microenvironment of gliomas. Lab Invest 97, 498-518 (2017).
15. Beier, C. P. et al. The cancer stem cell subtype determines immune infiltration of glioblastoma. Stem Cells Dev 21, 2753-2761 (2012).
16. Lottaz, C. et al. Transcriptional profiles of CD133+ and CD133-glioblastoma-derived cancer stem cell lines suggest different cells of origin. Cancer research 70, 2030-2040 (2010).
17. Shibao, S. et al. Metabolic heterogeneity and plasticity of glioma stem cells in a mouse glioblastoma model. Neuro-oncology 20, 343-354 (2018).
18. Nishimura, M. et al. Protection against natural killer cells by interferon-gamma treatment of K562 cells cannot be explained by augmented major histocompatibility complex class I expression. Immunology 83, 75-80 (1994).
19. Cerwenka, A., Baron, J. L. & Lanier, L. L. Ectopic expression of retinoic acid early inducible-1 gene (RAE-1) permits natural killer cell-mediated rejection of a MHC class I-bearing tumor in vivo. Proceedings of the National Academy of Sciences of the United States of America 98, 11521-11526 (2001).
20. Gras Navarro, A., Bjorklund, A. T. & Chekenya, M. Therapeutic potential and challenges of natural killer cells in treatment of solid tumors. Front Immunol 6, 202 (2015).
21. Vivier, E., Ugolini, S., Blaise, D., Chabannon, C. & Brossay, L. Targeting natural killer cells and natural killer T cells in cancer. Nat Rev Immunol 12, 239-252 (2012).
22. Zhang, H. et al. Activating signals dominate inhibitory signals in CD137L/IL-15 activated natural killer cells. Journal of immunotherapy 34, 187-195 (2011).
23. Tran Thang, N. N. et al. Immune infiltration of spontaneous mouse astrocytomas is dominated by immunosuppressive cells from early stages of tumor development. Cancer research 70, 4829-4839 (2010).
24. Kmiecik, J. et al. Elevated CD3+ and CD8+ tumor-infiltrating immune cells correlate with prolonged survival in glioblastoma patients despite integrated immunosuppressive mechanisms in the tumor microenvironment and at the systemic level. Journal of neuroimmunology 264, 71-83 (2013).
25. Viel, S. et al. TGF-beta inhibits the activation and functions of NK cells by repressing the mTOR pathway. Sci Signal 9, ra19 (2016).
26. Capper, D. et al. Biomarker and Histopathology Evaluation of Patients with Recurrent Glioblastoma Treated with Galunisertib, Lomustine, or the Combination of Galunisertib and Lomustine. Int J Mol Sci 18 (2017).
27. Brandes, A. A. et al. A Phase II randomized study of galunisertib monotherapy or galunisertib plus lomustine compared with lomustine monotherapy in patients with recurrent glioblastoma. Neuro-oncology 18, 1146-1156 (2016).
28. Zhang, M. et al. Blockade of TGF-beta signaling by the TGFbetaR-I kinase inhibitor LY2109761 enhances radiation response and prolongs survival in glioblastoma. Cancer research 71, 7155-7167 (2011).
29. Gonzalez-Junca, A. et al. Autocrine TGFbeta Is a Survival Factor for Monocytes and Drives Immunosuppressive Lineage Commitment. Cancer Immunol Res 7, 306-320 (2019).
30. Liu, Z., Kuang, W., Zhou, Q. & Zhang, Y. TGF-beta1 secreted by M2 phenotype macrophages enhances the sternness and migration of glioma cells via the SMAD⅔ signalling pathway. Int J Mol Med 42, 3395-3403 (2018).
31. Kessenbrock, K., Plaks, V. & Werb, Z. Matrix metalloproteinases: regulators of the tumor microenvironment. Cell 141, 52-67 (2010).
32. Gialeli, C., Theocharis, A. D. & Karamanos, N. K. Roles of matrix metalloproteinases in cancer progression and their pharmacological targeting. FEBS J 278, 16-27 (2011).
33. Wang, M., Wang, T., Liu, S., Yoshida, D. & Teramoto, A. The expression of matrix metalloproteinase-2 and-9 in human gliomas of different pathological grades. Brain Tumor Pathol 20, 65-72 (2003).
34. Mamuya, F. A. & Duncan, M. K. aV integrins and TGF-beta-induced EMT: a circle of regulation. J Cell Mol Med 16, 445-455 (2012).
35. Roth, P. et al. Integrin control of the transforming growth factor-beta pathway in glioblastoma. Brain 136, 564-576 (2013).
36. Yu, J. et al. The CD9, CD81, and CD151 EC2 domains bind to the classical RGD-binding site of integrin alphavbeta3. Biochem J 474, 589-596 (2017).
37. Wan, Y. Y. & Flavell, R. A. ‘Yin-Yang’ functions of transforming growth factor-beta and T regulatory cells in immune regulation. Immunol Rev 220, 199-213 (2007).
38. Wipff, P. J. & Hinz, B. Integrins and the activation of latent transforming growth factor beta1-an intimate relationship. Eur J Cell Biol 87, 601-615 (2008).
39. Gu, X., Niu, J., Dorahy, D. J., Scott, R. & Agrez, M. V. Integrin alpha(v)beta6-associated ERK2 mediates MMP-9 secretion in colon cancer cells. Br J Cancer 87, 348-351(2002).
40. Thomas, G. J. et al. alpha v beta 6 Integrin upregulates matrix metalloproteinase 9 and promotes migration of normal oral keratinocytes. J Invest Dermatol 116, 898-904 (2001).
41. Dutta, A. et al. alphavbeta6 integrin is required for TGFbeta1-mediated matrix metalloproteinase2 expression. Biochem J 466, 525-536 (2015).
42. Deryugina, E. I. et al. MT1-MMP initiates activation of pro-MMP-2 and integrin alphavbeta3 promotes maturation of MMP-2 in breast carcinoma cells. Exp Cell Res 263, 209-223 (2001).
43. Kim, E. S., Kim, M. S. & Moon, A. TGF-beta-induced upregulation of MMP-2 and MMP-9 depends on p38 MAPK, but not ERK signaling in MCF10A human breast epithelial cells. Int J Oncol 25, 1375-1382 (2004).
44. Hsieh, H. L., Wang, H. H., Wu, W. B., Chu, P. J. & Yang, C. M. Transforming growth factor-beta1 induces matrix metalloproteinase-9 and cell migration in astrocytes: roles of ROS-dependent ERK- and JNK-NF-kappaB pathways. J Neuroinflammation 7, 88 (2010).
45. Canazza, A. et al. Increased migration of a human glioma cell line after in vitro CyberKnife irradiation. Cancer Biol Ther 12, 629-633 (2011).
46. Herbertz, S. et al. Clinical development of galunisertib (LY2157299 monohydrate), a small molecule inhibitor of transforming growth factor-beta signaling pathway. Drug Des Devel Ther 9, 4479-4499 (2015).
47. Bogdahn, U. et al. Targeted therapy for high-grade glioma with the TGF-beta2 inhibitor trabedersen: results of a randomized and controlled phase IIb study. Neuro-oncology 13, 132-142 (2011).
48. Li, L. et al. A novel immature natural killer cell subpopulation predicts relapse after cord blood transplantation. Blood Adv 3, 4117-4130 (2019).
49. Muftuoglu, M. et al. Allogeneic BK Virus-Specific T Cells for Progressive Multifocal Leukoencephalopathy. N Engl J Med 379, 1443-1451 (2018).
50. Van Gassen, S. et al. FlowSOM: Using self-organizing maps for visualization and interpretation of cytometry data. Cytometry A 87, 636-645 (2015).
51. Kordasti, S. et al. Deep-phenotyping of Tregs identifies an immune signature for idiopathic aplastic anemia and predicts response to treatment. Blood (2016).
52. Jiang, H. et al. Examination of the therapeutic potential of Delta-24-RGD in brain tumor stem cells: role of autophagic cell death. J Natl Cancer Inst 99, 1410-1414 (2007).
53. Pollard, S. M. et al. Glioma stem cell lines expanded in adherent culture have tumor-specific phenotypes and are suitable for chemical and genetic screens. Cell stem cell 4, 568-580 (2009).
54. Gabrusiewicz, K. et al. Glioblastoma stem cell-derived exosomes induce M2 macrophages and PD-L1 expression on human monocytes. Oncoimmunology 7, e1412909 (2018).
55. Lal, S. et al. An implantable guide-screw system for brain tumor studies in small animals. J Neurosurg 92, 326-333 (2000).
56. Shah, N. et al. Antigen presenting cell-mediated expansion of human umbilical cord blood yields log-scale expansion of natural killer cells with anti-myeloma activity. PLoS One 8, e76781 (2013).
57. Pino, P. A. & Cardona, A. E. Isolation of brain and spinal cord mononuclear cells using percoll gradients. J Vis Exp (2011).

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. A composition comprising two or more of (a), (b), (c), and (d):
(a) one or both of (1) and (2):
(1) one or more compounds that disrupt expression or activity of transforming growth factor (TGF)-beta receptor 2 (TGFBR2);
(2) natural killer (NK) cells comprising a disruption of expression or activity of TGFBR2 endogenous to the NK cells;
(b) one or both of (1) and (2):
(1) one or more compounds that disrupt expression or activity of glucocorticoid receptor (GR);
(2) natural killer (NK) cells comprising a disruption of expression or activity of GR endogenous to the NK cells;
(c) one or more integrin inhibitors; and
(d) one or more TGF-beta inhibitors,
wherein the two or more of (a), (b), (c), and (d) may or may not be in the same formulation.

2. The composition of claim 1, wherein the NK cells are expanded NK cells.

3. The composition of claim 1 or 2, wherein the one or more compounds that disrupt expression or activity of TGFBR2 and/or GR comprises nucleic acid, peptide, protein, small molecule, or a combination thereof.

4. The composition of claim 3, wherein the nucleic acid comprises siRNA, shRNA, anti-sense oligonucleotides, or guide RNA for CRISPR.

5. The composition of any one of claims 1-4, wherein the one or more integrin inhibitors comprises nucleic acid, peptide, protein, small molecule, or a combination thereof.

6. The composition of claim 5, wherein the integrin inhibitor is a small molecule.

7. The composition of claim 5, wherein the integrin inhibitor is a protein that is an antibody.

8. The composition of claim 7, wherein the antibody is a monoclonal antibody.

9. The composition of any one of claims 1-8, wherein the integrin inhibitor inhibits more than one integrin.

10. The composition of any one of claims 1-9, wherein the integrin inhibitor is cilengitide; Abciximab; Eptifibatide; Tirofiban; Natalizumab; Vedolizumab; etaracizumab; abegrin; CNTO95; ATN-161; vipegitide; MK0429; E7820; Vitaxin; 5247; PSK1404; S137; HYD-1; abituzumab; Intetumumab; RGD-containing linear or cyclic peptide; Lifitegrast; Leukadherin-1; A 205804; A 286982; ATN 161; BIO 1211; BIO 5192; BMS 688521; BOP; BTT 3033; E 7820; Echistatin; GR 144053 trihydrochloride; MNS; Obtustatin; P11; R-BC154; RGDS peptide; TC-I 15; TCS 2314, or a combination thereof.

11. The composition of any one of claims 1-10, wherein the one or more TGF-beta inhibitors comprises nucleic acid, peptide, protein, small molecule, or a combination thereof.

12. The composition of claim 11, wherein the TGF-beta inhibitor is a small molecule.

13. The composition of claim 11, wherein the TGF-beta inhibitor is an antibody.

14. The composition of claim 13, wherein the antibody is a monoclonal antibody.

15. The composition of claim 1, wherein the composition comprises, consists essentially of, or consists of (a)(1) and/or (a)(2) and (b)(1) and/or (b)(2).

16. The composition of claim 1, wherein the composition comprises, consists essentially of, or consists of (a)(1), and (c).

17. The composition of claim 1, wherein the composition comprises, consists essentially of, or consists of (a)(1) and (d).

18. The composition of claim 1, wherein the composition comprises, consists essentially of, or consists of (a)(2) and (c).

19. The composition of claim 1, wherein the composition comprises, consists essentially of, or consists of (a)(2) and (d).

20. The composition of claim 1, wherein the composition comprises, consists essentially of, or consists of (b)(1), and (c).

21. The composition of claim 1, wherein the composition comprises, consists essentially of, or consists of (b)(1) and (d).

22. The composition of claim 1, wherein the composition comprises, consists essentially of, or consists of (b)(2) and (c).

23. The composition of claim 1, wherein the composition comprises, consists essentially of, or consists of (b)(2) and (d).

24. The composition of claim 1, wherein the composition comprises, consists essentially of, or consists of (c) and (d).

25. The composition of claim 1, wherein the composition comprises, consists essentially of, or consists of (a)(1), (a)(2), (b), and (c).

26. The composition of claim 1, wherein the composition comprises, consists essentially of, or consists of (a)(1), (a)(2), and (b).

27. The composition of claim 1, wherein the composition comprises, consists essentially of, or consists of (a)(1), (a)(2), and (c).

28. The composition of claim 1, wherein the composition comprises, consists essentially of, or consists of (a)(1), (a)(2), and (d).

29. The composition of claim 1, wherein the composition comprises, consists essentially of, or consists of (a)(1), (b), and (c).

30. The composition of claim 1, wherein the composition comprises, consists essentially of, or consists of (a)(2), (b), and (c).

31. The composition of claim 1, wherein the composition comprises, consists essentially of, or consists of (b)(1), (b)(2), (c), and (d).

32. The composition of claim 1, wherein the composition comprises, consists essentially of, or consists of (b)(1), (b)(2), and (c).

33. The composition of claim 1, wherein the composition comprises, consists essentially of, or consists of (b)(1), (b)(2), and (d).

34. The composition of claim 1, wherein the composition comprises, consists essentially of, or consists of (b)(1), (c), and (d).

35. The composition of claim 1, wherein the composition comprises, consists essentially of, or consists of (b)(2), (c), and (d).

36. The composition of claim 1, wherein two or more of (a)(1), (a)(2), (b)(1), (b)(2), (c), and (d) are in the same formulation.

37. The composition of claim 1, wherein two or more of (a)(1), (a)(2), (b)(1), (b)(2), (c), and (d) are in different formulations.

38. The composition of any one of claims 1-37, wherein the NK cells are cord blood NK cells or are derived therefrom.

39. The composition of any one of claims 1-36, wherein in (a)(2) and/or (b)(2) the NK cells are NK cells engineered to express one or more chimeric antigen receptors and/or one or more synthetic T cell receptors.

40. The composition of claim 39, wherein the chimeric antigen receptor and/or synthetic T cell receptor targets one or more tumor antigens.

41. The composition of claim 40, wherein the tumor antigen is associated with glioblastoma.

42. The composition of any one of claims 1-41, wherein in (a)(2) and/or (b)(2) the NK cells are engineered to express one or more heterologous cytokines.

43. The composition of any one of claims 1-42, further comprising NK cells that are not the NK cells of (a)(2) and/or (b)(2).

44. The composition of any one of claims 1-43, wherein the composition is comprised in a pharmaceutically acceptable carrier.

45. A method of killing cancer cells in an individual, comprising the step of delivering to the individual a therapeutically effective amount of the composition of any one of claims 1-44.

46. The method of claim 45, wherein the cancer cells are cancer stem cells.

47. The method of claim 45 or 46, wherein the cancer is a hematological cancer or comprises a solid tumor.

48. The method of any one of claims 45-47, wherein the cancer is glioblastoma.

49. The method of claim 48, wherein the cancer is glioblastoma and the cancer cells comprise cancer stem cells.

50. The method of any one of claims 45-49, wherein the NK cells are autologous or allogeneic with respect to the individual.

51. The method of any one of claims 45-50, wherein the NK cells are peripheral blood NK cells, cord blood NK cells, or NK cell lines that are allogeneic with respect to the individual.

52. The method of any one of claims 45-51, wherein the NK cells were cryopreserved before the delivering step.

53. The method of any one of claims 45-52, wherein the composition comprises an effective amount of one or both of (a)(1) and (a)(2); and (b).

54. The method of any one of claims 45-52, wherein the composition comprises an effective amount of one or both of (a)(1) and (a)(2); and (c).

55. The method of any one of claims 45-52, wherein the composition comprises an effective amount of (b) and (c).

56. The method of any one of claims 42-52, wherein the individual is delivered an additional cancer therapy.

57. The method of claim 53, wherein the additional cancer therapy comprises surgery, radiation, chemotherapy, hormone therapy, immunotherapy, or a combination thereof.

58. A kit, comprising the compositions or any one of claims 1-44, and/or one or more reagents to generate the compositions, housed in a suitable container.
